(19) United States
(12) Patent Application Publication
(10) Pub. No.: US 2010/0292229 A1
(43) Pub. Date: Nov. 18, 2010

(54) TRYPHOSTIN-ANALOGS FOR THE TREATMENT OF CELL PROLIFERATIVE DISEASES

(75) Inventors: Nicholas J Donato, Sugar Land, TX (US); David Maxwell, Pearland, TX (US); Moshe Talpaz, Houston, TX (US); William Bornmann, Missouri City, TX (US); Zhenghong PENG, Missouri City, TX (US); Ashutosh Pal, Houston, TX (US); Dongmei Han, Houston, TX (US); Shimei Wang, Houston, TX (US); Geoffrey Bartholomeusz, Houston, TX (US); Vaibhav Kapuria, Houston, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of the University of Texas System, Austin, TX (US)

(21) Appl. No.: 12/307,088

(22) PCT Filed: Jul. 2, 2007
(86) PCT No.: PCT/US2007/072693

§ 371 Date: Apr. 14, 2010

Related U.S. Application Data
(60) Provisional application No. 60/806,426, filed on Jun. 30, 2006.
(60) Provisional application No. 60/826,052, filed on Sep. 18, 2006.

(51) Int. Cl. A61K 31/538; C07D 213/46; A61K 31/44; C07C 255/44; A61K 31/275; C07D 233/64; A61K 31/4174; C07D 215/14; A61K 31/47; C07D 211/06; A61K 31/451; C07D 209/20; A61K 31/4045; C07D 333/24; A61K 31/381; C07D 265/36; A61P 35/00; A61P 19/02; A61P 9/10; A61P 17/06

(52) U.S. Cl. 514/230.5; 546/330; 514/357; 558/401; 514/521; 548/336.1; 514/400; 546/175; 514/311; 546/247; 514/315; 548/492; 514/419; 549/69; 514/438; 544/105

(57) ABSTRACT

The present invention concerns compounds and their use to treat cell proliferative diseases such as cancer. In general aspects, compounds of the present invention are tyrphostin-like in structure. Compounds of the present invention, in certain embodiments, display significant potency by causing, for example, inhibition of Stat3 activation, reduction in c-myc protein levels and/or induction of apoptosis in tumor cells. In general aspects, compounds of the present invention induce one or more of these activities at nanomolar concentrations and typically function through a unique mechanism involving the induction of stress granules that bind specific signaling molecules and prevent them from participating in signal transduction and oncogenesis.

The present application claims priority to U.S. Provisional Patent Application Ser. No. 60/806,426 filed Jun. 30, 2006, and U.S. Provisional Patent Application Ser. No. 60/826,052 filed Sep. 18, 2006. The entire text of these disclosures are specifically incorporated by reference herein without disclaimer.

### BACKGROUND OF THE INVENTION

1. Field of the Invention

The present invention relates generally to the treatment of cell proliferative diseases such as cancer. More particularly, it concerns tyrphostin and tyrphostin-like compounds useful for the treatment of cell proliferative diseases such as cancer, methods of synthesis of these compounds, and methods of treatment employing these compounds.

2. Description of Related Art

Signaling proteins are key components of the cellular circuitry that link internal and external stimuli to change in cell morphology and gene expression and are highly regulated in normal cells. In pathologies, including cancer, regulation of signaling proteins is disrupted by gene mutations and chromosomal translocations resulting in unregulated growth and survival, tumor metastases and blocked differentiation. Reducing expression or returning signaling proteins to their inactive state reverses many of the characteristics associated with cancer and these proteins serve as effective targets for cancer and other therapies.

AG490 (CAS No. 34036-52-5), shown below, is a kinase inhibitor that inhibits Janus kinase 2/Signal transducer and activator of transcription-3 (Jak2/Stat3) signaling:

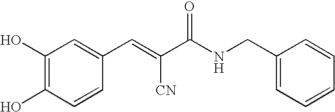

Jak2/STAT3 signaling pathways participate in the progression of a variety of malignancies. STAT3 is constitutively activated in pancreatic carcinoma, glioblastoma multiforme, and squamous cell carcinoma of the head and neck, among others, and its activation has been shown to affect VEGF expression, angiogenesis, tumor growth, and metastasis in vivo. Targeted inhibition of the Jak/Stat pathway with AG490 inhibits tumor cell growth and increases sensitivity to apoptotic stimuli; thus, inhibitors of this pathway likely represent potential therapeutics for cancer therapy (Catlett-Falcone et al., 1999; Alas and Bonavida, 2003; Burdelya et al., 2002). Because IL-6 promotes survival and proliferation of certain cancerous cell lines through the phosphorylation of STAT3 (Bharti et al., Verma et al., Kerr et al.), kinase inhibitors similar to AG490 have potential as anti-cancer drugs.

AG490 is often structurally classified as a tyrphostin. U.S. Pat. No. 6,596,828 and U.S. Application Publ. No. 2003/0013748 describe compounds that have structural similarity with AG490.

Unfortunately, AG490 has limited activity in animal studies and must be used at high concentrations (˜50 to 100 μM) to achieve inhibition of Jak2/Stat3 signaling and anti-tumor effects, and this low potency of AG490 is insufficient to warrant clinical investigation of this compound for the treatment of cancer (Burdelya et al., 2002; Meydan et al., 1996; Constantin et al., 1998). Thus a need exists for therapeutics that exhibit strong anti-proliferative effects through a similar mechanism at lower therapeutic concentrations.

### SUMMARY OF THE INVENTION

The present invention overcomes limitations in the art by providing compounds that display improved pharmacological profiles (e.g., increased potency) when compared with AG490 and other tyrphostin-related compounds. Compounds of the present invention comprise small molecules that, generally speaking, have been designed, synthesized and/or demonstrated to sequester or reduce the stability of signaling proteins such as c-myc proto-oncogene (“c-myc”), Stat complexes (e.g., stat3) and the tyrosine kinases Jak2 and BCR-ABL, through a novel mechanism. The c-myc proto-oncogene is frequently overexpressed, rearranged, or mutated in many malignancies (Hallek et al., 1998; Selvanayagam et al., 1988; Jernberg-Wiklund et al., 1992; Kuehl et al., 1997). These compounds, in certain embodiments, induce anti-tumor effects by altering the cellular distribution and/or stability of these important signaling proteins through, for example, the formation of stress granules that recruit and capture these proteins, preventing them from participating in their oncogenic activities. Accordingly, the present invention involves, in certain embodiments, compounds that have utility as antitumor and/or chemotherapeutic drugs, methods of synthesizing these compounds, and methods of using these compounds to treat patients with cancer.

In general, compounds of the present invention represent modified forms of the chemical structure of tyrphostin, shown below:

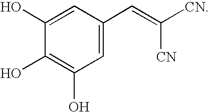

Certain compounds of the present invention were subject to extensive structure-activity and computational analysis to define structural elements that improve their target-specific activity and anti-tumor efficacy in vitro and in vivo. Certain compounds showing heightened potency were tested against known signaling targets for activity. Of those compounds tested, certain compounds induced apoptosis of tumor cells at nanomolar concentrations (e.g., IC50 values equaling, for example, 250 nM and 400 nM) and/or caused inhibition of Jak2/Stat3 signaling and/or destabilizing of c-myc protein in tumor cells. Thus, in a general aspect, the present invention describes a class of small molecules with nanomolar activity against multiple tumors and a novel mechanism of inhibitory action against critical signaling proteins that are essential to cancer cells.

Based on earlier studies of tyrphostin derivatives, these compounds may, in certain embodiments, exhibit anti-tumor activity against a wide range of tumor types, such as leukemia, lymphoma, multiple myeloma, head and neck, prostate and melanoma, while having limited toxicity against normal cells (dermal fibrosis). Nielsen et al. 1997, Catlett-Falcone et al. 1999, De Vos et al. 2000, Epling-Burnette et al. 2001, Garcia et al. 2001, Liu et al. 2002, Alas and Bonavida 2003, Toyonaga et al. 2003, Barton et al., 2004, Lai et al. 2005, Shouda et al. 2006, Lee et al. 2006, Niu et al. 2002.

Accordingly, the compound of formula (I), which displays some elements of AG490 and some elements of tyrphostin, represents certain compounds of the present invention:

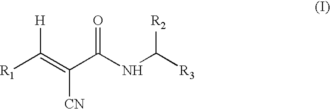

wherein:
R₁ is aryl or

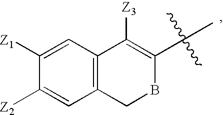

wherein
   Z₁ is H or OH;
   Z₂ is H, chloro, or —OCH₃;
   Z₃ is H or chloro; and
   B is C or O;
R₂ is selected from the group consisting of R₄ and R₅-R₆, wherein:
   R₄ is selected from the group consisting of H, alkyl and aryl;
   R₅ is alkyl; and
   R₆ is selected from the group consisting of aryl, acyl, acyloxy, hydroxy and biotinyl; or
R₂ taken together with R₃ forms

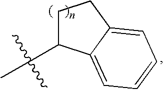

wherein n is 1-3; and
R₃ is aryl.

The present invention specifically does not encompass any of the compounds selected from the group consisting of:

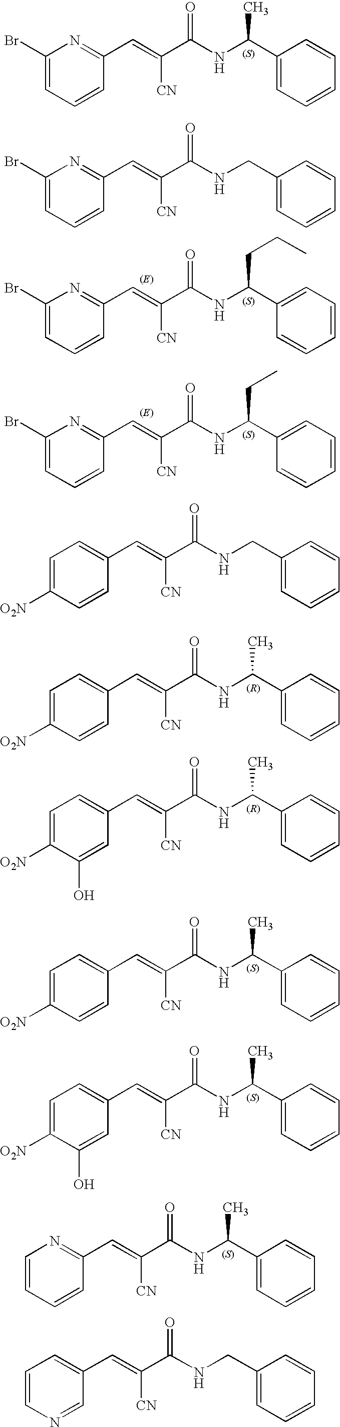

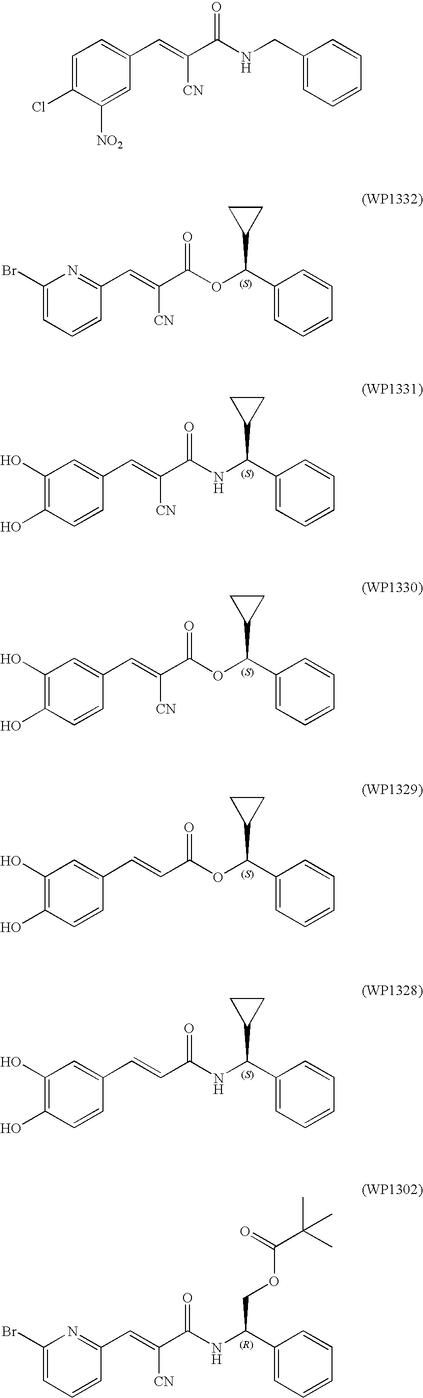

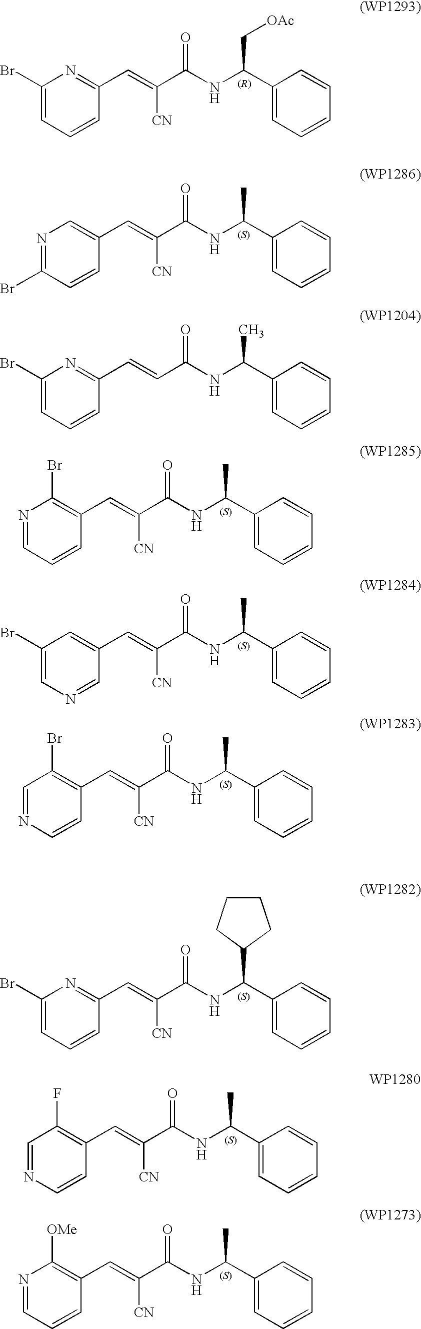

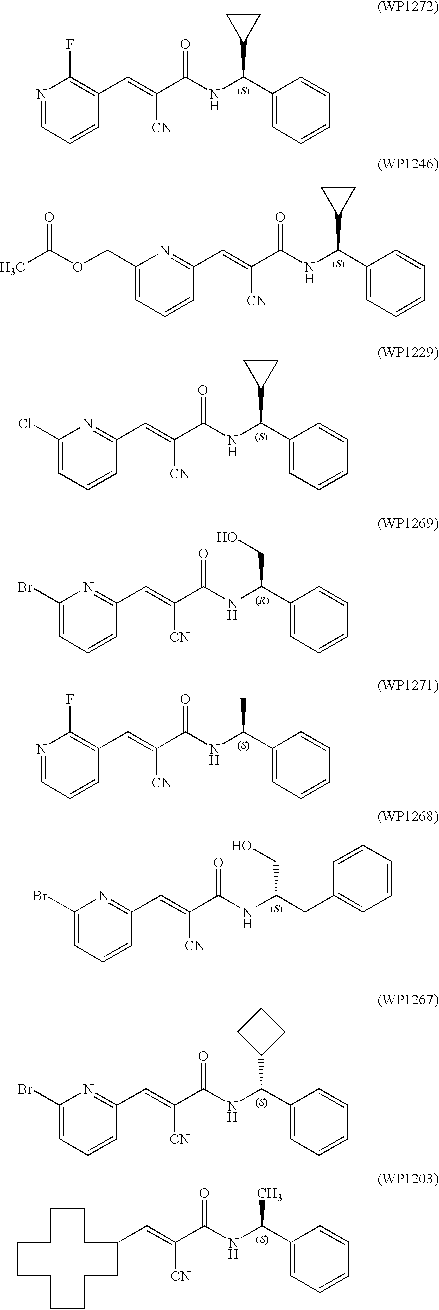

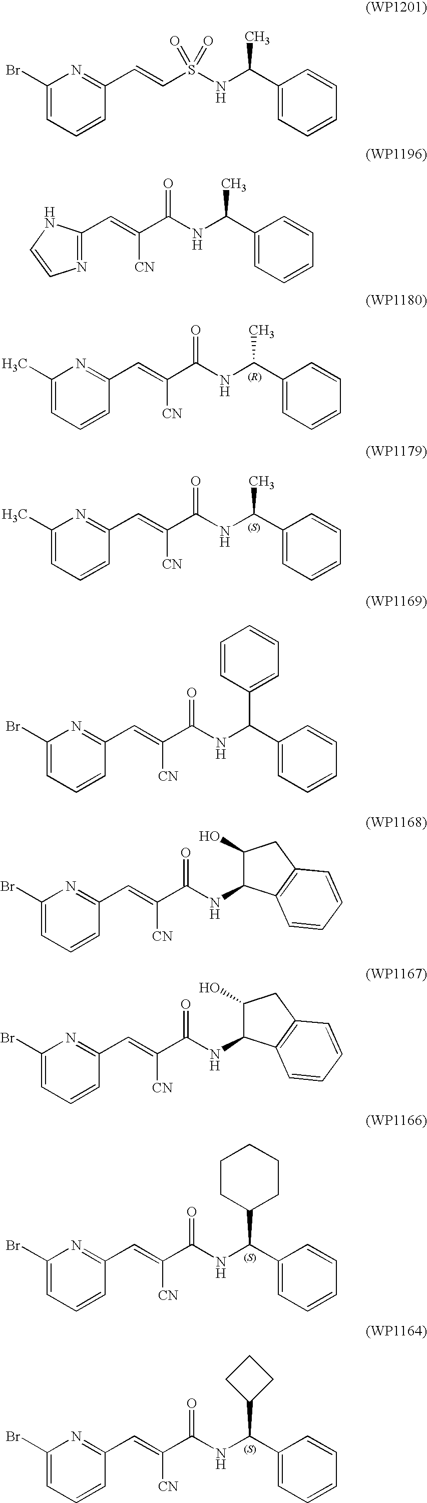

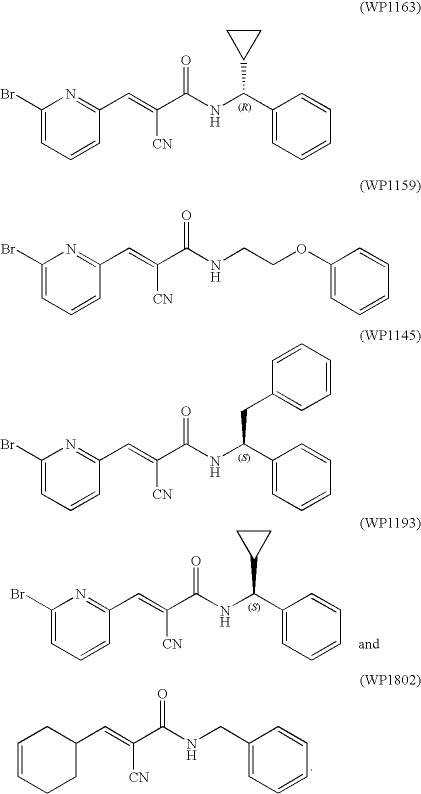

In certain embodiments, compounds of formula (I), (II), (III), (IV), (V), and/or (VI) are contemplated. In certain embodiments, any combination of formulas (I), (II), (III), (IV), (V), or (VI) are contemplated as well (e.g., certain embodiments contemplate compounds of formula (II) and compounds of formula (VI)). Some specific compounds that are encompassed by one of formula (I), (II), (III), (IV), (V), or (VI) may be encompassed by one or more of formulas (I), (II), (III), (IV), (V), or (VI). Some specific compounds may be encompassed only by one formula. In certain embodiments, a formula of (I), (II), (III), (IV), (V), or (VI) may exclude any one or more of formulas (I), (II), (III), (IV), (V), or (VI). Any of formulas (I), (II), (III), (IV), (V), or (VI) may exclude specific compounds as well, such as those listed above as specifically not encompassed by the present invention.

In certain aspects of the present invention, a compound of formula (II) is contemplated:

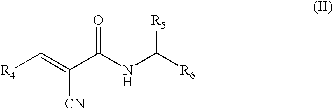

wherein:
R₄ is furanyl, thienyl, indolyl, ortho-bromopyridyl,

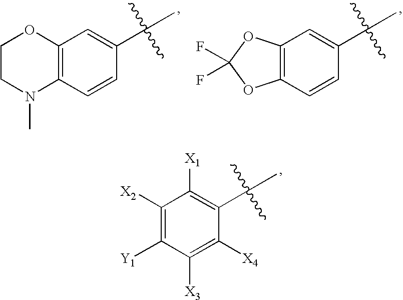

wherein:
   X₁, X₂, X₃ and X₄ are each independently H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, halo, hydroxy, amino, azido, mercapto, nitro, or cyano; and
   Y₁ is H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, amino, azido, mercapto, or cyano; or

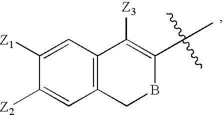

wherein
   Z₁ is H or OH;
   Z₂ is H, chloro, or —OCH₃;
   Z₃ is H or chloro; and
   B is C or O;
R₅ is H, alkyl, phenyl, or biotinyl; and
R₆ is phenyl or methylfuranyl.

In certain embodiments regarding compounds of formula (II), if R₄ is ortho-bromopyridyl, then R₅ is biotinyl.

Non-limiting examples of compounds of formula (II) include the following:

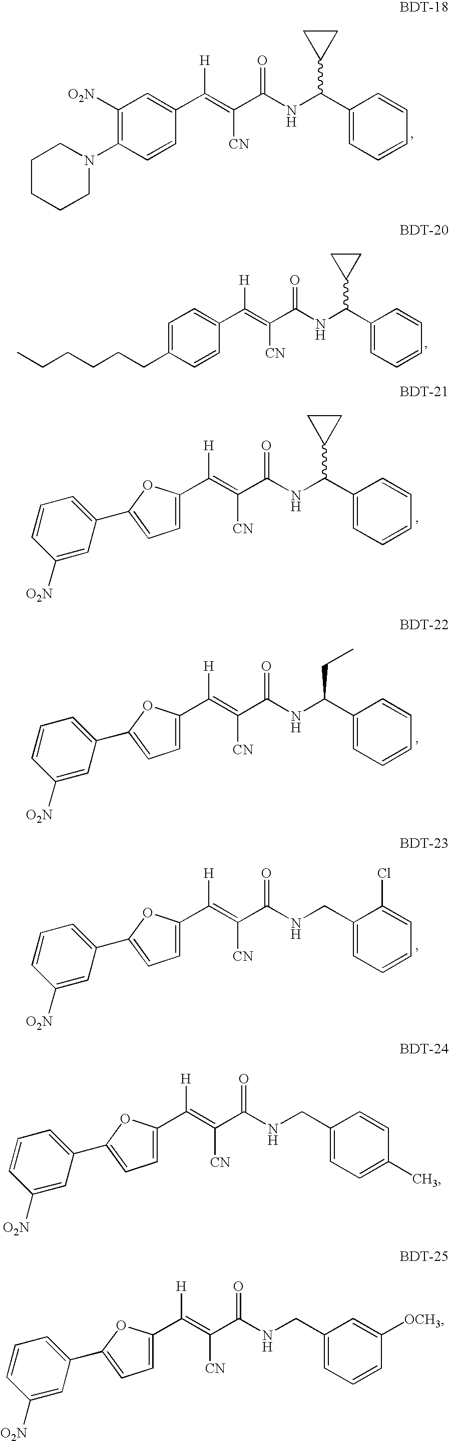

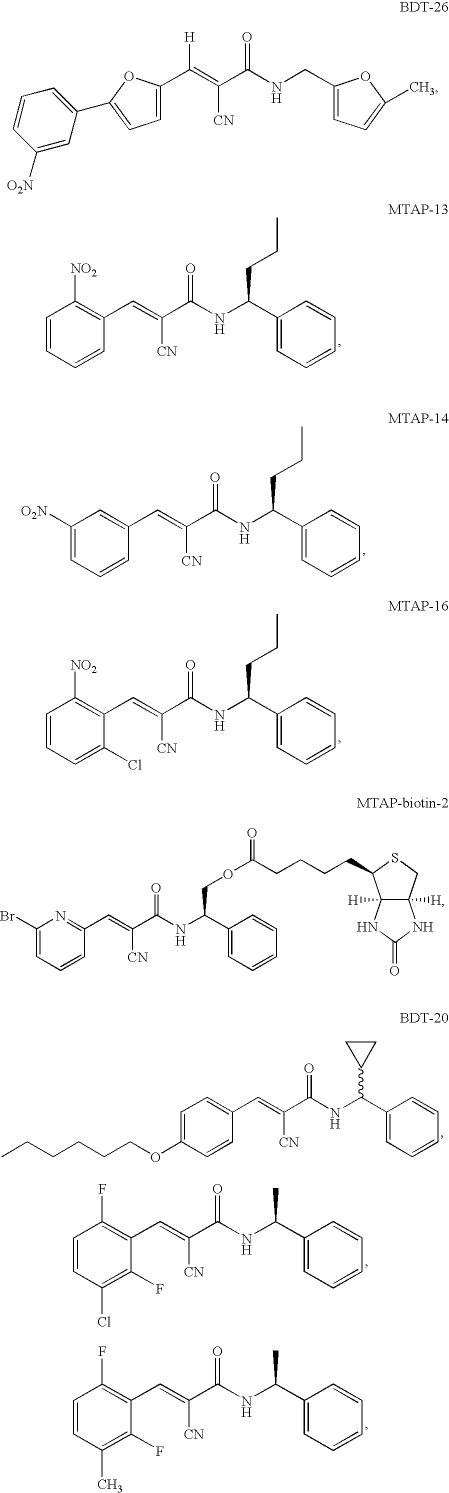

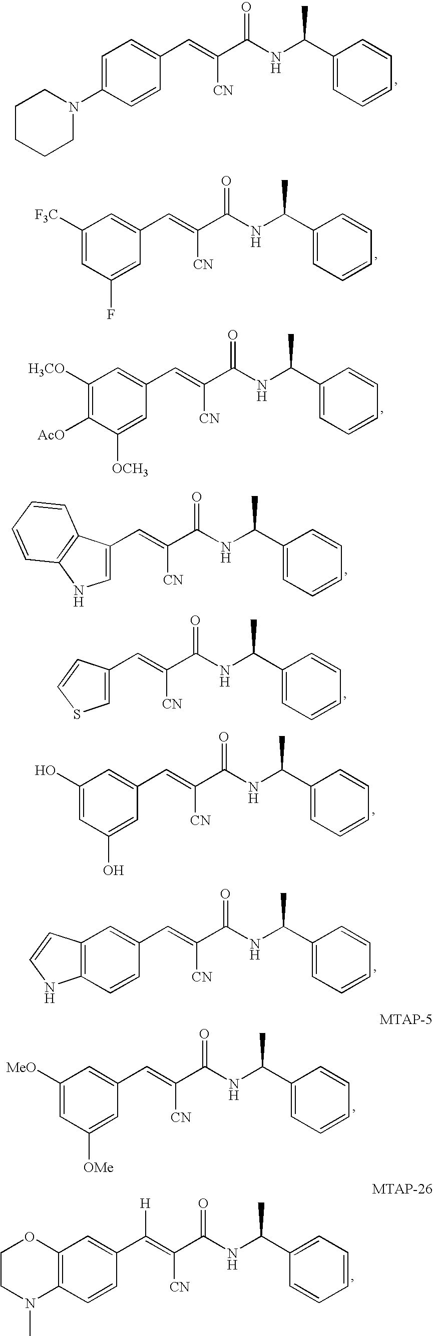

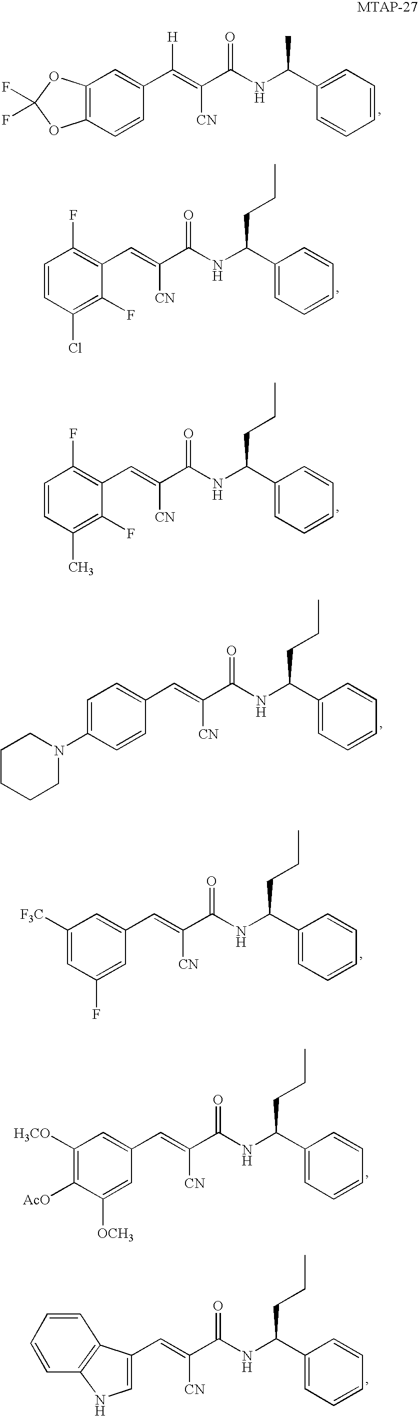

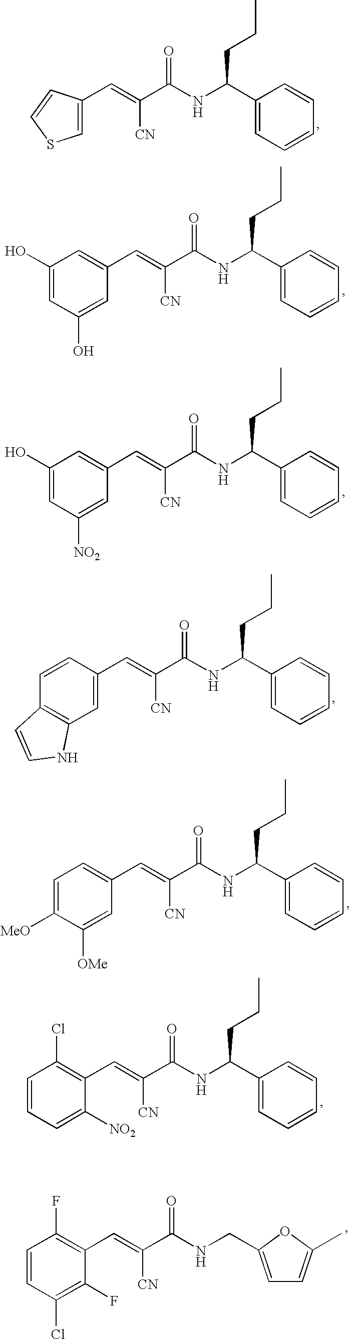

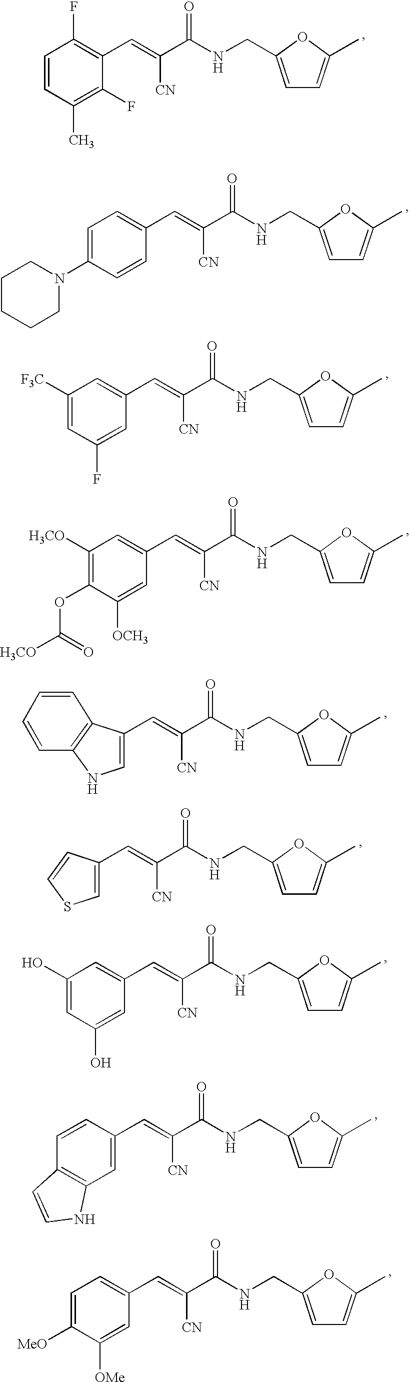

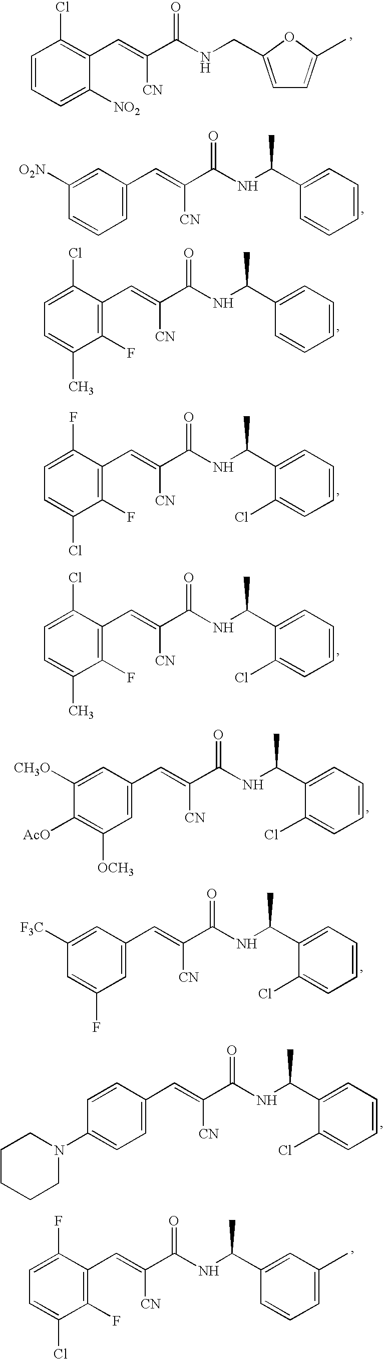

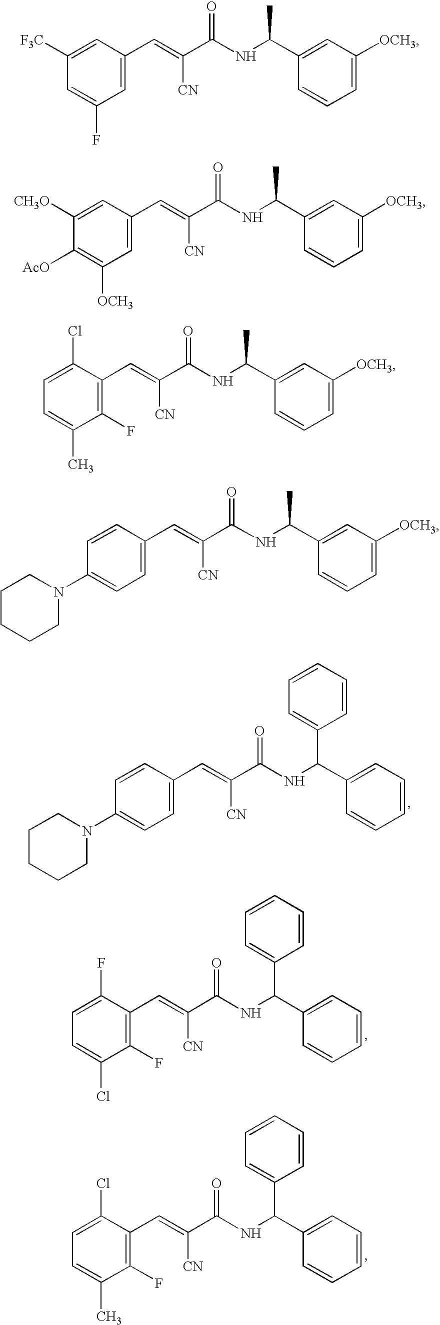

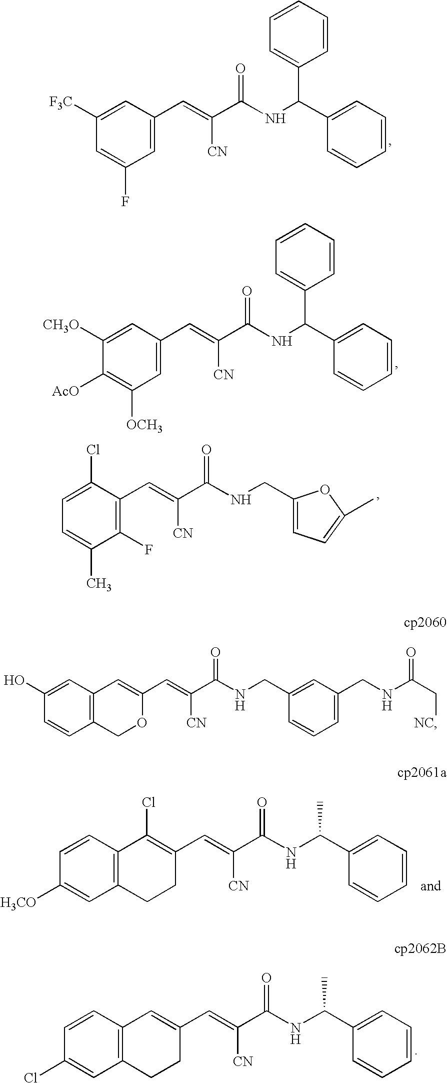

In certain aspects of the present invention, a compound of formula (III) is contemplated:

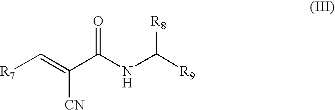

wherein:
R₇ is imidazolyl or:

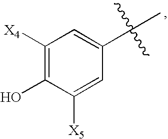

wherein:
   X₄ and X₅ are each independently H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, halo, hydroxy, amino, azido, mercapto, nitro, or cyano;
R₈ is H, alkyl, or phenyl; and
R₉ is phenyl or furanyl.

In certain embodiments regarding compounds of formula III, when R₇ is imidazolyl and R₈ is —CH₃, then R₉ is not —C₆H₅; and/or when X₄ is hydroxy, X₅ is H and R₈ is

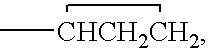

then R₉ is not —C₆H₅.

Non-limiting examples of compounds of formula (III) include the following:

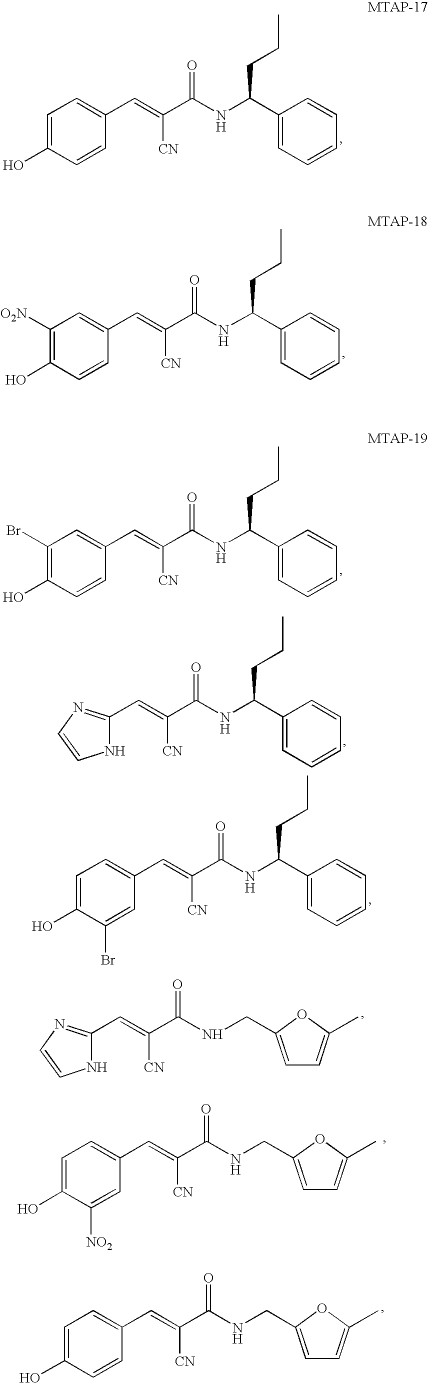

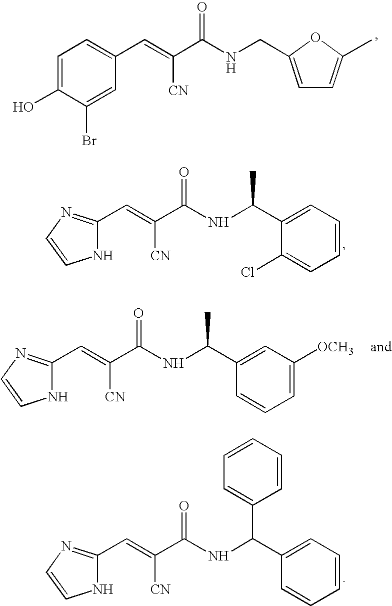

In certain aspects of the present invention, a compound of formula (IV) is contemplated:

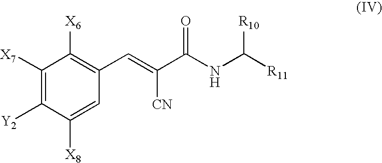

wherein:
R₁₀ is H, alkyl, or phenyl;
R₁₁ is phenyl or furanyl;
X₆ and X₇ are each independently H or nitro;
X₈ is H, halogen, or nitro; and
Y₂ is halogen or nitro.

In certain embodiments regarding compounds of formula (IV), when with the provisos that when Y₂ is nitro, X₆, X₇ and X₈ are each H and R₁₀ is either H or —CH₃, then R₁₁ is not —C₆H₅; when Y₂ is nitro, X₆ and X₇ are each H, X₈ is hydroxy and R₁₀ is —CH₃, then R₁₁ is not —C₆H₅; and/or when Y₂ is chloro, X₆ and X₇ are each H, X₈ is nitro and R₁₀ is H, then R₁₁ is not —C₆H₅.

Non-limiting examples of compounds of formula (IV) include:

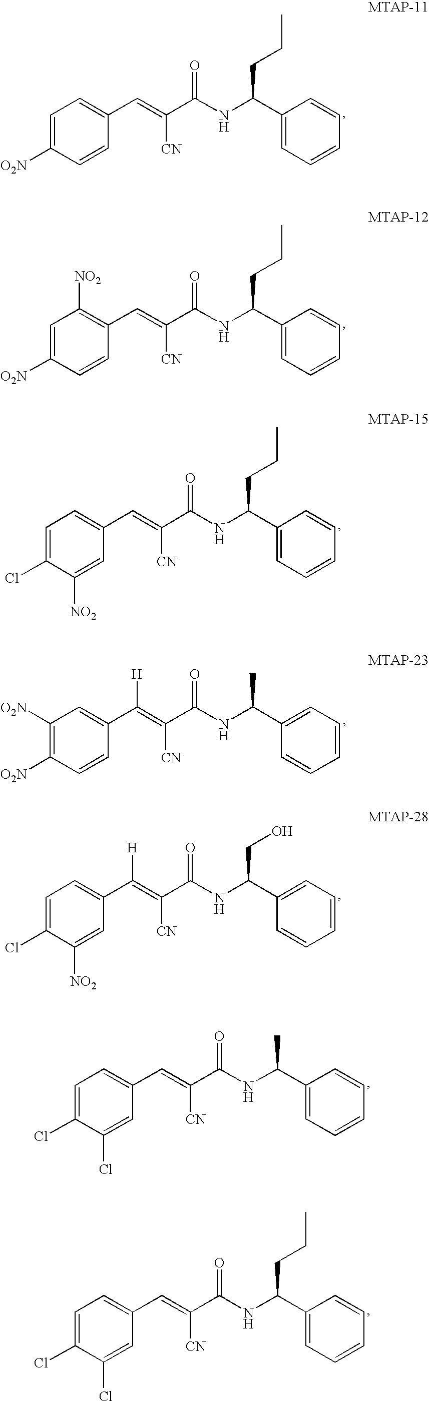

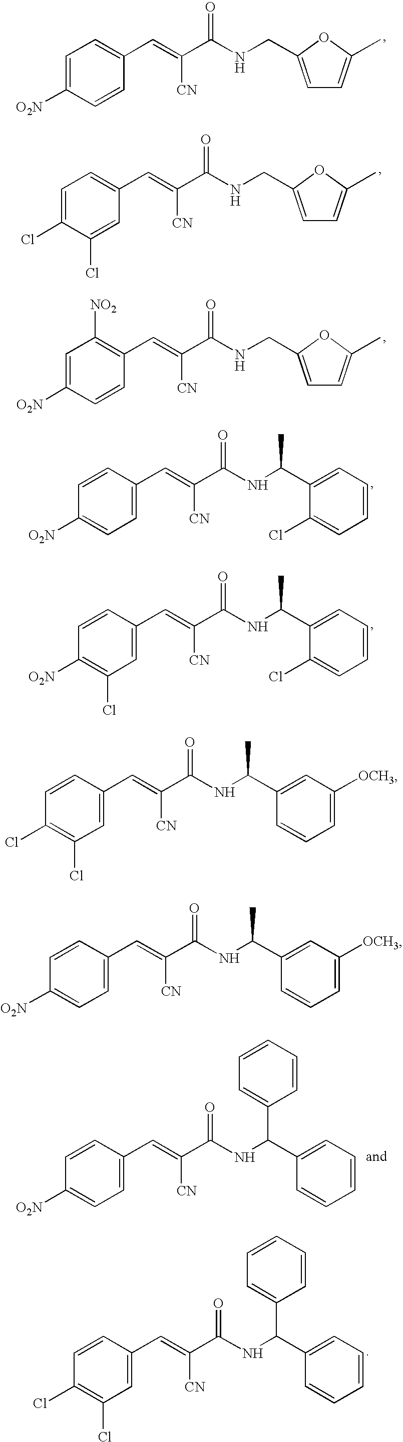

In certain aspects of the present invention, a compound of formula (V) is contemplated:

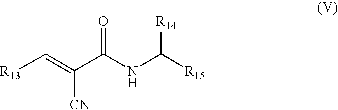

wherein:
R₁₃ is quinolinyl or ortho-bromopyridyl;
R₁₄ is selected from the group consisting of R_{A} and R_{B}-R_{C}, wherein:
   R_{A} is selected from the group consisting of H, alkyl and phenyl;
   R_{B} is alkyl; and
   R_{C} is selected from the group consisting of phenyl, furanyl, acyl, acyloxy, hydroxy and biotinyl; or
R₁₄ taken together with R₁₅ forms

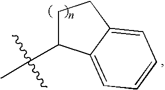

wherein n is 1-3; and
R₁₅ is furanyl or phenyl.

In certain embodiments regarding compounds of formula (V), when R₁₃ is ortho-bromopyridyl and R₁₅ is —C₆H₅, then R₁₄ is not H, —CH₃, —CH₂CH₃, —CH₂CH₂CH₃, —C₆H₅, —CH₂C₆H₅, —CH₂OH, —CH₂OAc, —CH₂OC(O)CH(CH₃)₃, or

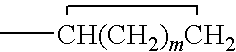

wherein m=1-3.

Non-limiting examples of compounds of formula (V) include:

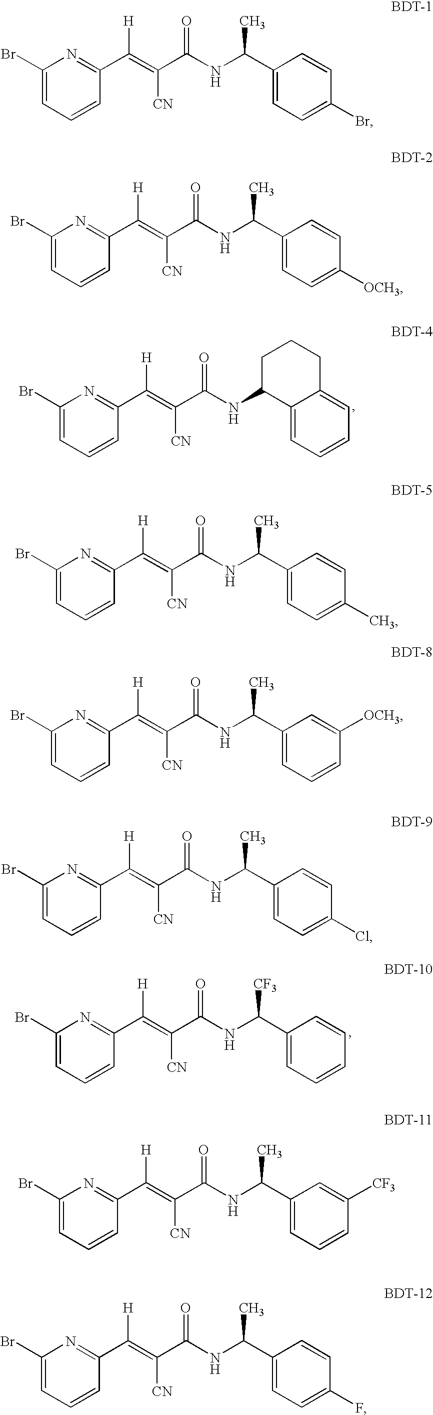

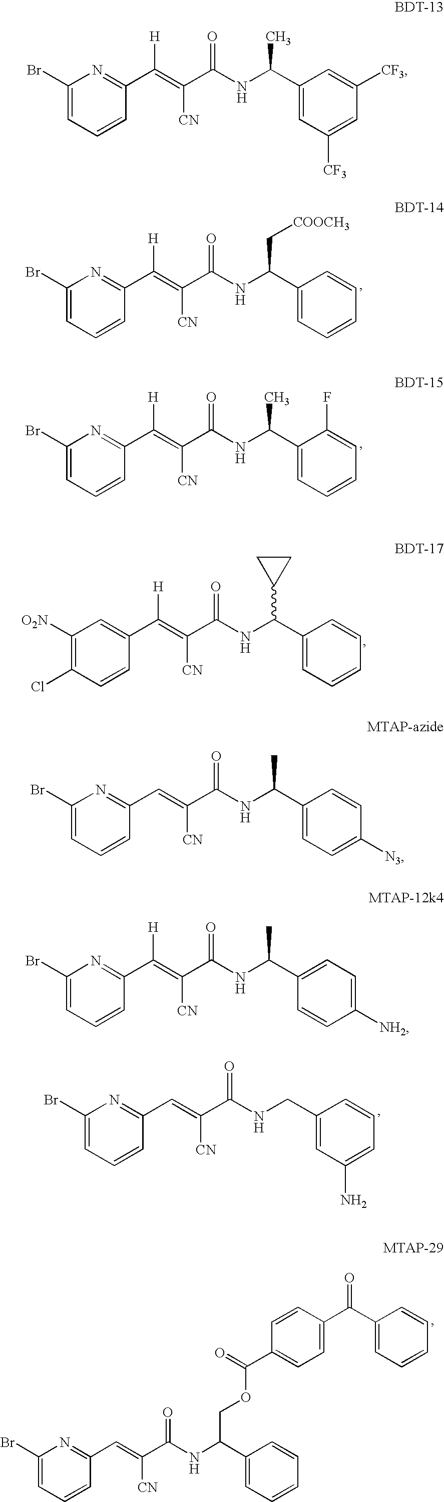

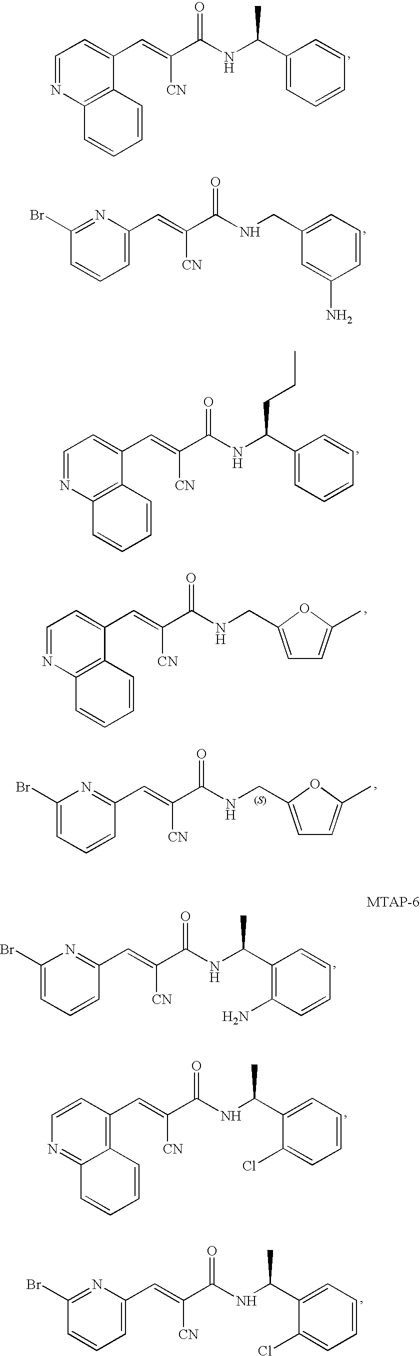

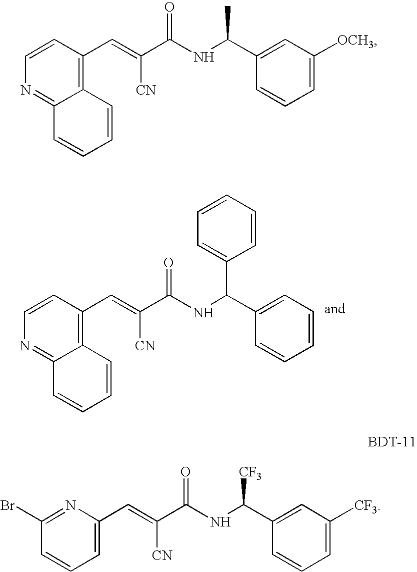

In certain aspects of the present invention, a compound of formula (VI) is contemplated:

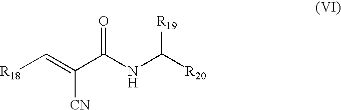

wherein:
R₁₈ is ortho-bromopyridyl or

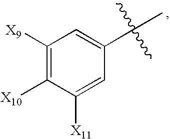

wherein:
   X₉ and X₁₁ are each independently H or nitro; and
   X₁₀ is H or chloro;
R₁₉ is —CH₃, —CH₂CH₃, —CH₂CH₂CH₃, —CH₂OH, —CH₂C₆H₅, —CH₂CO₂CH₃, —CH₂OC(O)CH₃, or

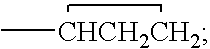

or
R₁₉ taken together with R₂₀ forms

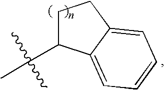

wherein n is 1-3; and
R₂₀ is

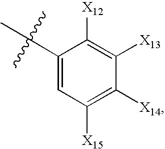

wherein:
   X₁₂ is H or fluoro;
   X₁₃ is H, —OCH₃, or fluoro;
   X₁₄ is H, —CH₃, bromo, chloro, fluoro, or —OCH₃; and
   X₁₅ is H or —CF₃.

In certain embodiments regarding compounds of formula (VI), when R₁₈ is ortho-bromopyridyl and R₂₀ is —C₆H₅, then R₁₉ is not —CH₃, —CH₂CH₃, —CH₂OH, or

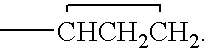

In particular embodiments regarding the compound of formula (II), R₁ may be selected from the group consisting of H, alkyl, alkoxy, acyl, N-piperidinyl,

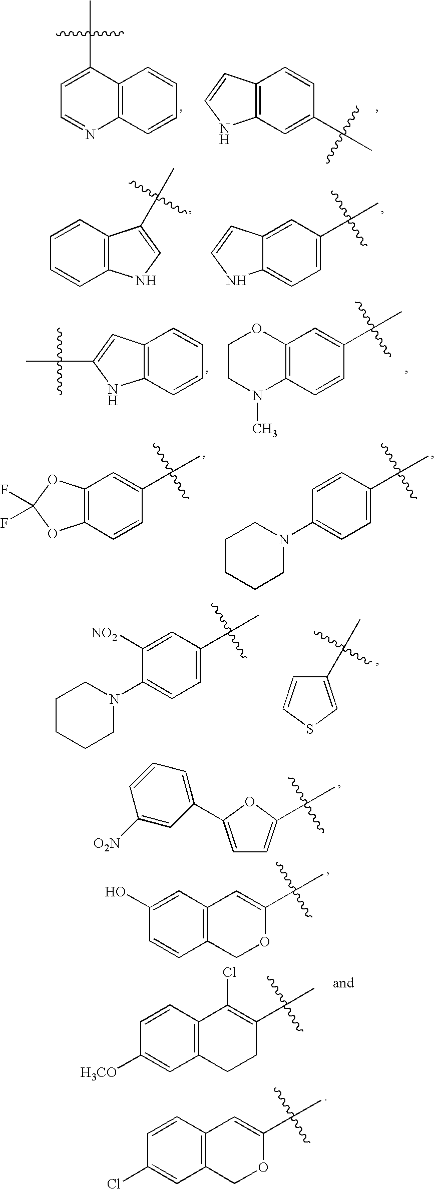

Regarding compounds of formula (II), Yᵢ may be selected from the group consisting of H, n-hexyl, —OC₆H₁₃, —OCO₂CH₃, —OCH₃ and —OAc. In particular embodiments, Y₁ may be H and X₁, X₂, X₃ and X₄ are each independently selected from the group consisting of H, halo, hydroxy, nitro, —OCH₃, —OAc and —OC(O)OCH₃. R₆ may be mono-, di-, or tri-substituted phenyl (e.g., C₆H₄OCH₃ is an example of a mono-substituted phenyl), in certain embodiments. In certain embodiments, R₅ is selected from the group consisting of H, —CH₃, —CH₂CH₃, —CH₂—CH₂CH₃,

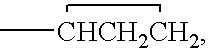

substituted or unsubstituted phenyl and methylfuranyl. R₅ may, in certain embodiments, be biotinyl.

In certain aspects of the present invention, the compound of formula (III) is contemplated. In certain embodiments, R₇ is mono- or di-substituted phenyl (e.g., C₆H₄OH, C₆H₃(OH)(NO₂). In certain embodiments X₄ and X₅ are independently selected from the group consisting of H, halo and nitro. In certain embodiments, R₈ is selected from the group consisting of H, lower alkyl and —C₆H₅. In certain embodiments, R₉ is selected from the group consisting of —C₆H₅, —C₆H₄Cl, —C₆H₄OCH₃ and methylfuranyl.

In certain aspects of the present invention, the compound of formula (IV) is contemplated. In certain embodiments, X₆, X₇ and X₈ are each independently selected from the group consisting of H, halogen and nitro. For example, X₆, X₇ and X₈ may each independently be H or Cl. In certain embodiments, X₆, X₇ and X₈ are each independently H or NO₂. In certain embodiments, R₁₀ is selected from the group consisting of H, —CH₃, —CH₂CH₂CH₃, —CH₂OH and —C₆H₅. R₁₁ may, in certain embodiments, be mono-substituted phenyl or furanyl, (e.g., —C₆H₄OCH₃—C₆H₄Cl, or methylfuranyl (e.g., 2-methylfuranyl)).

In certain aspects of the present invention, the compound of formula (V) is contemplated. In certain embodiments, R₁₃ is

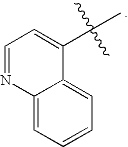

In certain embodiments, R₁₄ is selected from the group consisting of H, lower alkyl and phenyl. R_{C} may, in certain embodiments, be selected from the group consisting of —CO₂CH₃, —OC(O)CH₃, —OC(O)benzophenone and —C₆H₅. R₁₅ may be selected from the group consisting of mono- or di-substituted phenyl and methylfuranyl (e.g., 2-methylfuranyl), in certain embodiments. For example, R₁₅ may be mono- or di-substituted with a substituent selected from the group consisting of H, —CH₃, —CF₃, halo, —OCH₃, azido and amino.

In certain aspects of the present invention, the compound of formula (VI) is contemplated. In certain embodiments, R₁₉ is selected from the group consisting of —CH₃, —CH₂CH₃ and —CH₂CH₂CH₃. R₁₈ is ortho-bromopyridyl in certain embodiments. X₁₀ may be H in certain embodiments. In certain embodiments, X₉, X₁₀ and X₁₁ are each H. In certain embodiments, R₂₀ is selected from the group consisting of —C₆H₅ and mono- and di-substituted phenyl.

In certain embodiments, any compound of the present invention may be comprised in a pharmaceutically acceptable excipient, diluent, or vehicle. Also, any compound of the present invention may be substantially free from other optical isomers (e.g., enantiomers), in certain embodiments.

Another aspect of the present invention concerns a method of inducing stress granules that bind to and prevent one or more signaling molecules from participating in signal transduction and oncogenesis comprising contacting any one or more of compounds of the present invention. In preferred embodiments, the one or more signaling molecules are selected from a group consisting of c-myc, Stat3, Jak2 and BCR-ABL.

Another aspect of the present invention concerns a method of treating a cell proliferative disease comprising administering to a subject an amount of a first compound of the present invention effective to treat the cell proliferative disease in the subject, wherein the first compound is a compound of the present invention. The subject may be a mammal, and the mammal may be a human. The first compound may be comprised in a pharmaceutically acceptable excipient, diluent, or vehicle. The cell proliferative disease may be cancer. The cancer may be melanoma, non-small cell lung, small cell lung, lung, hepatocarcinoma, retinoblastoma, astrocytoma, glioblastoma, leukemia, blood, brain, skin, eye, tongue, gum, neuroblastoma, head, neck, breast, pancreatic, renal, bone, testicular, ovarian, mesothelioma, cervical, gastrointestinal, lymphoma, colon, or bladder cancer.

The cell proliferative disease may be rheumatoid arthritis, inflammatory bowel disease, osteoarthritis, leiomyomas, adenomas, lipomas, hemangiomas, fibromas, vascular occlusion, restenosis, atherosclerosis, a pre-neoplastic lesion, carcinoma in situ, oral hairy leukoplakia, or psoriasis.

In certain embodiments, c-myc, Jak2, Stat3 and/or BCL-ABL expression or activation is reduced in a cell of the subject. Other signaling protein activation may also be reduced. The first compound may be administered in combination with a therapeutically relevant amount of a second compound. The second compound may be an anti-cancer compound. The first compound may be administered in combination with a surgery, a radiation therapy, or a gene therapy.

In more specific embodiments, compounds of the present invention may include structures such as:

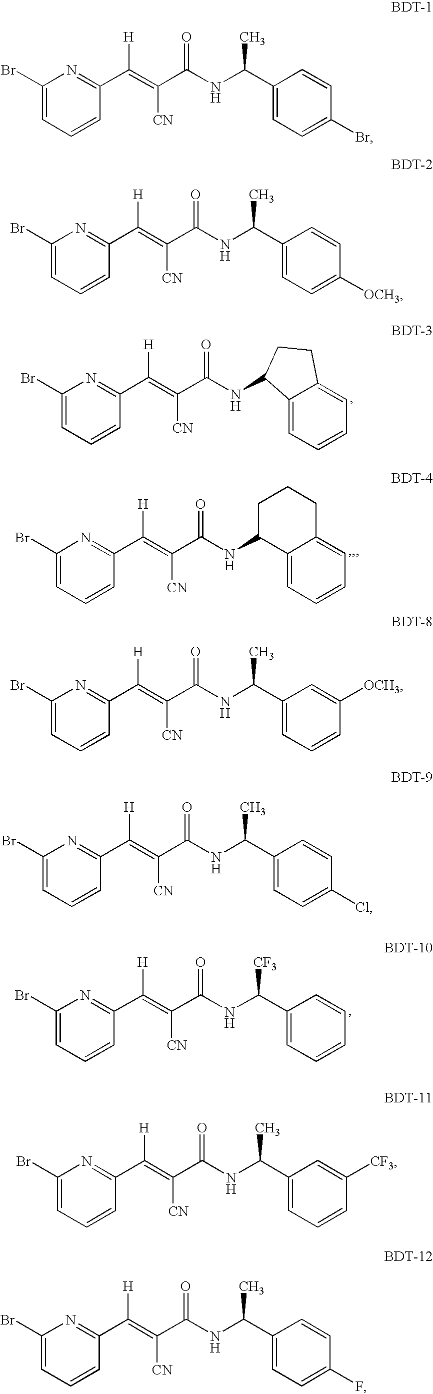

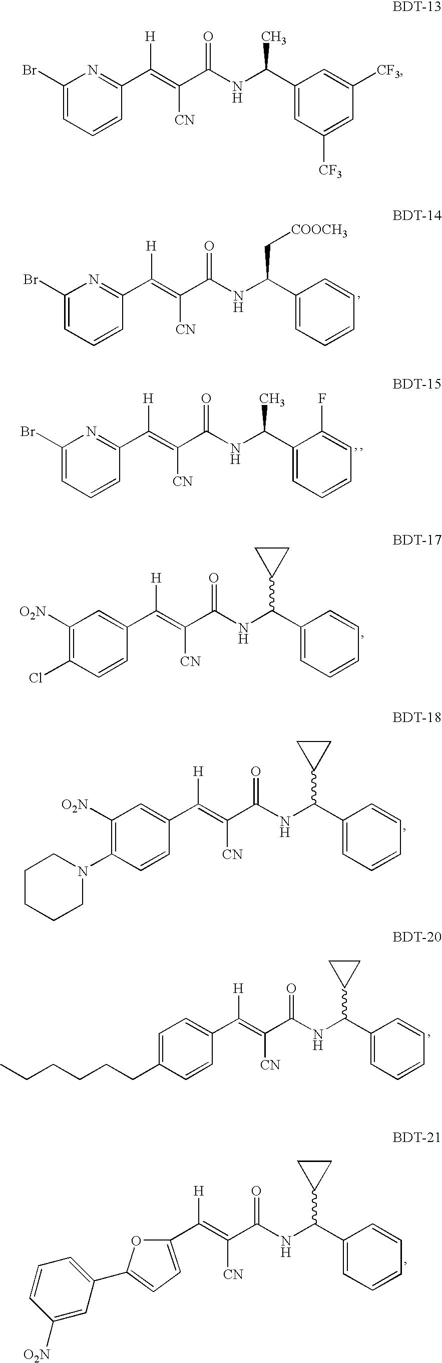

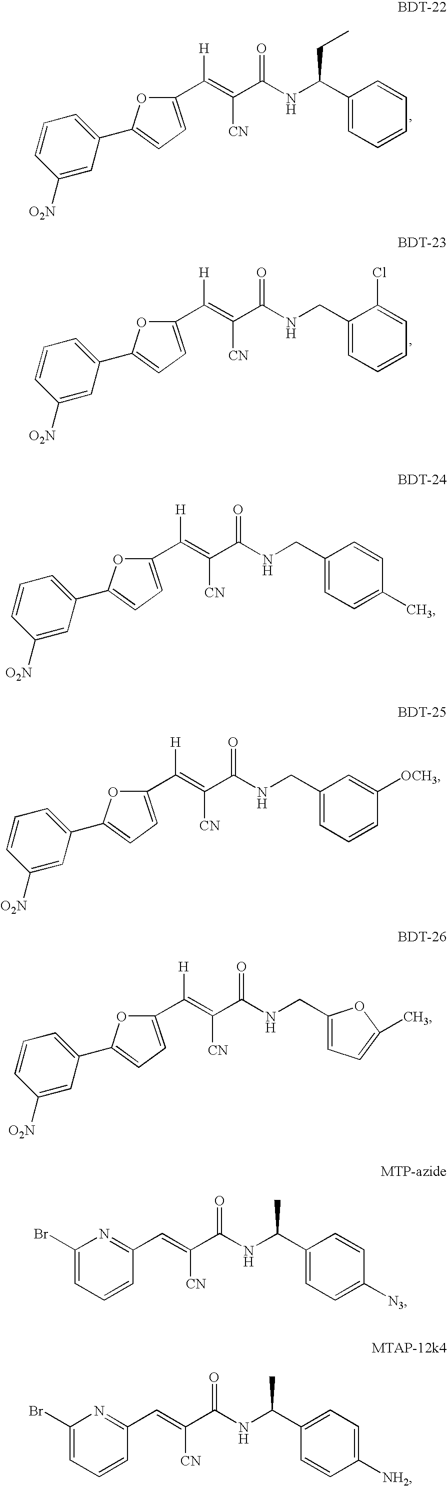

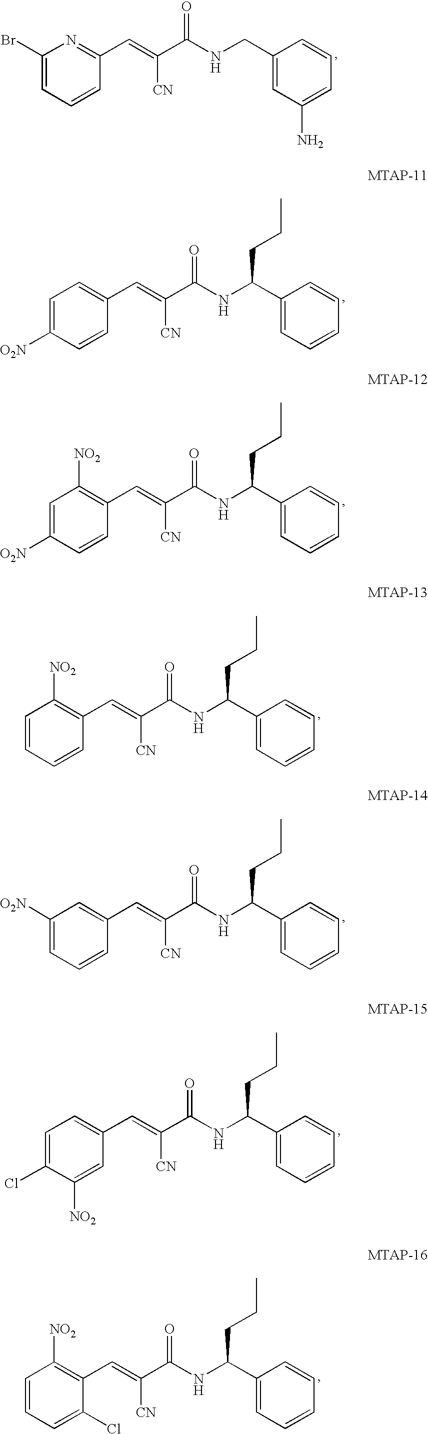

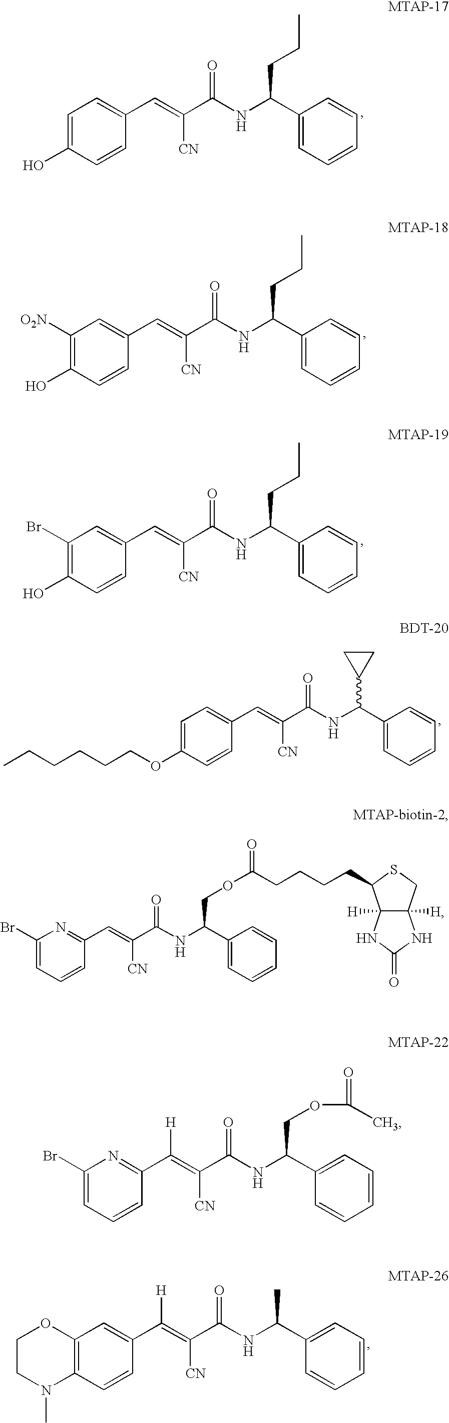

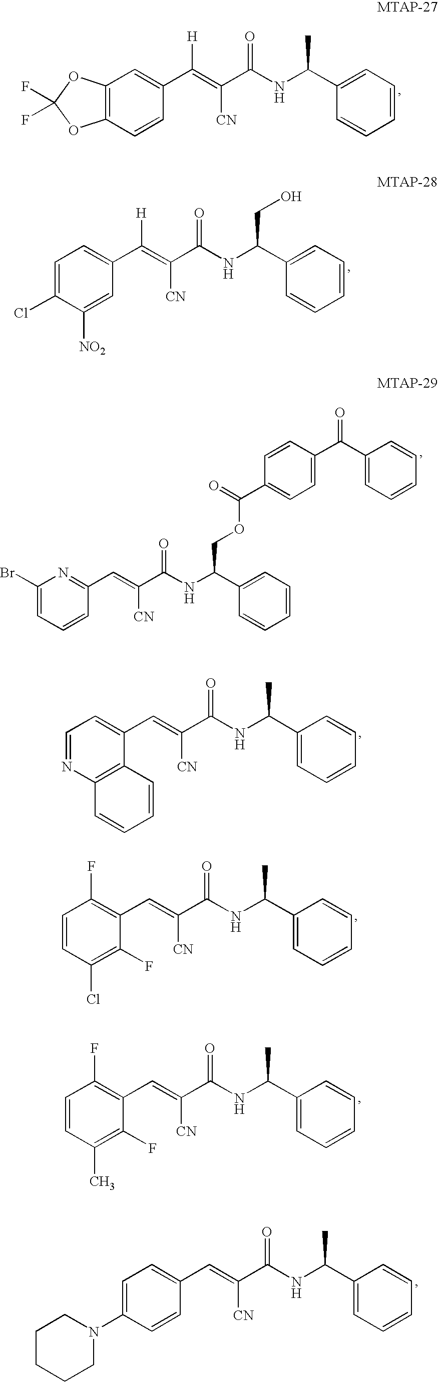

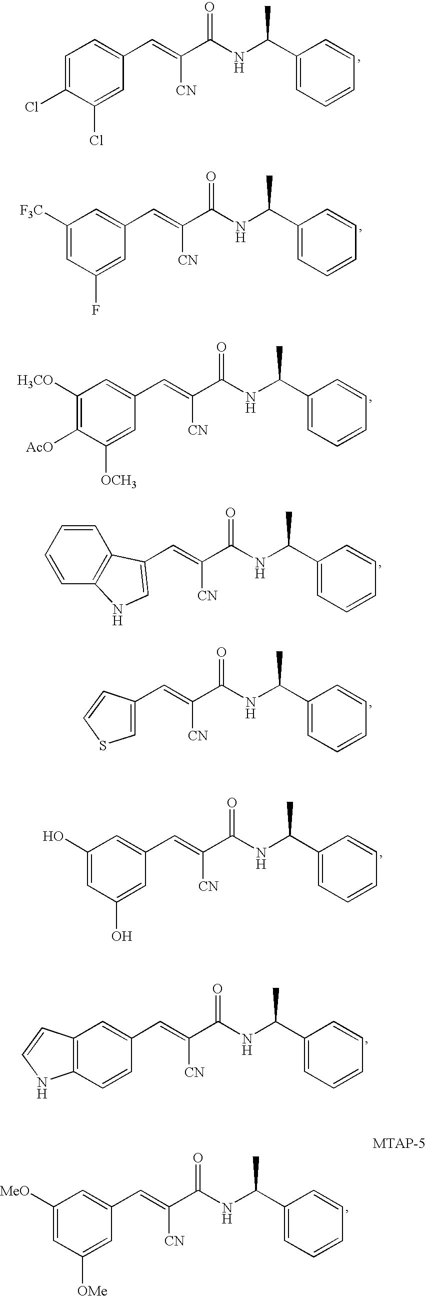

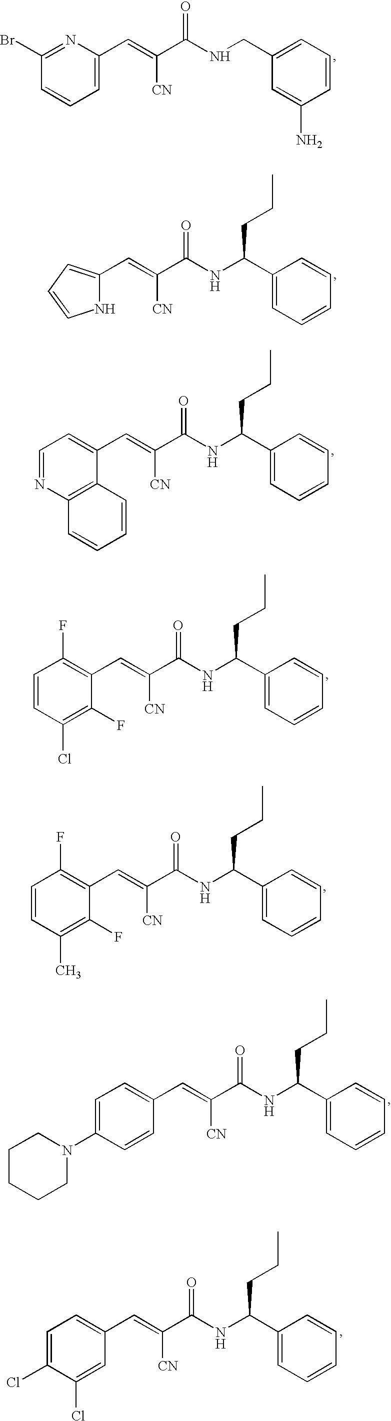

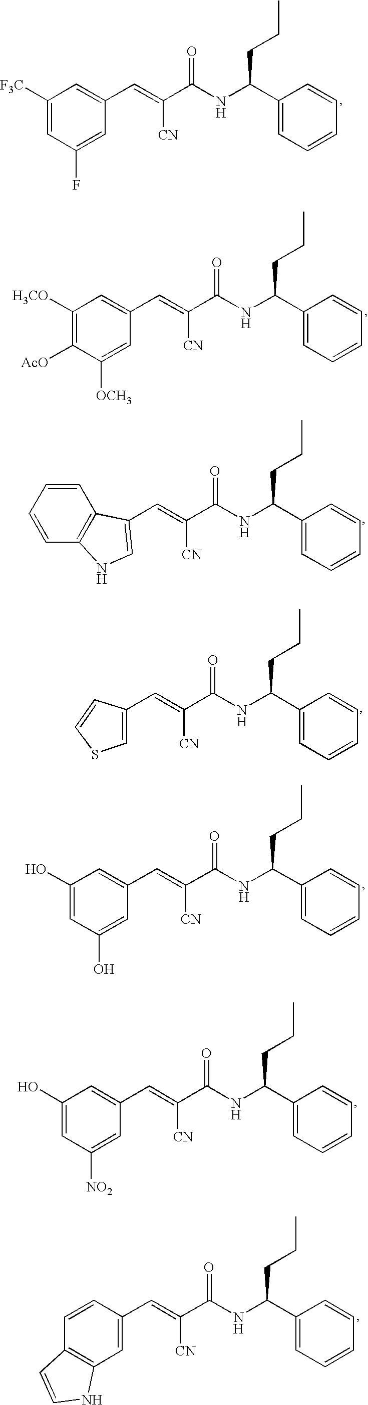

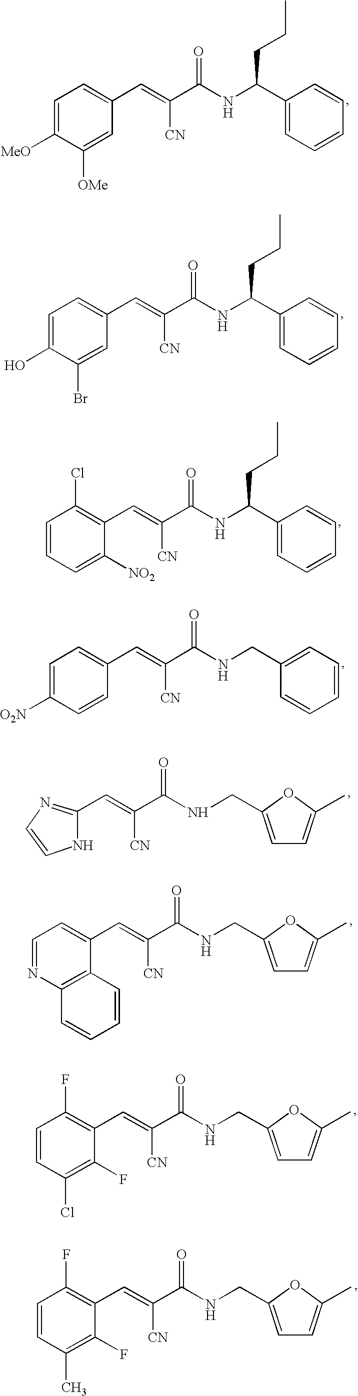

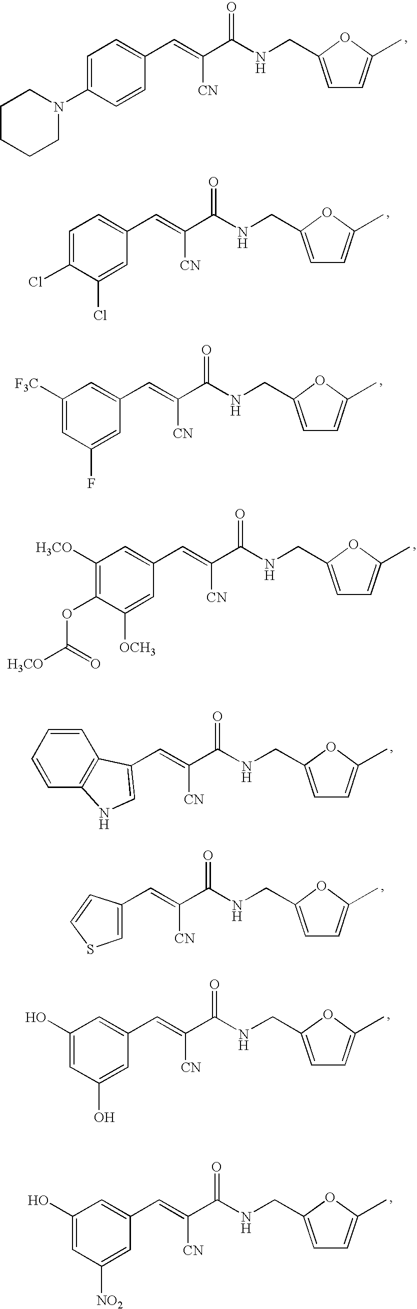

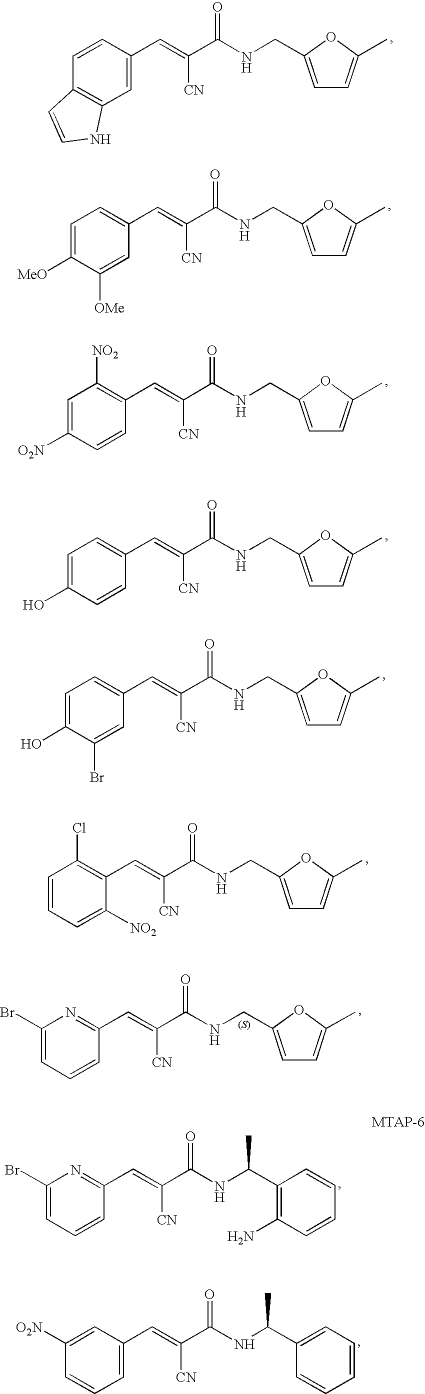

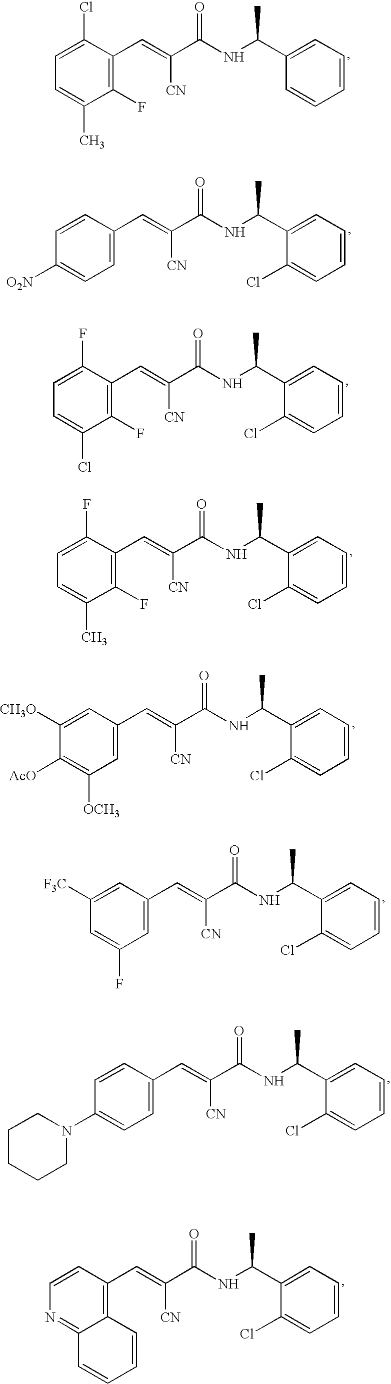

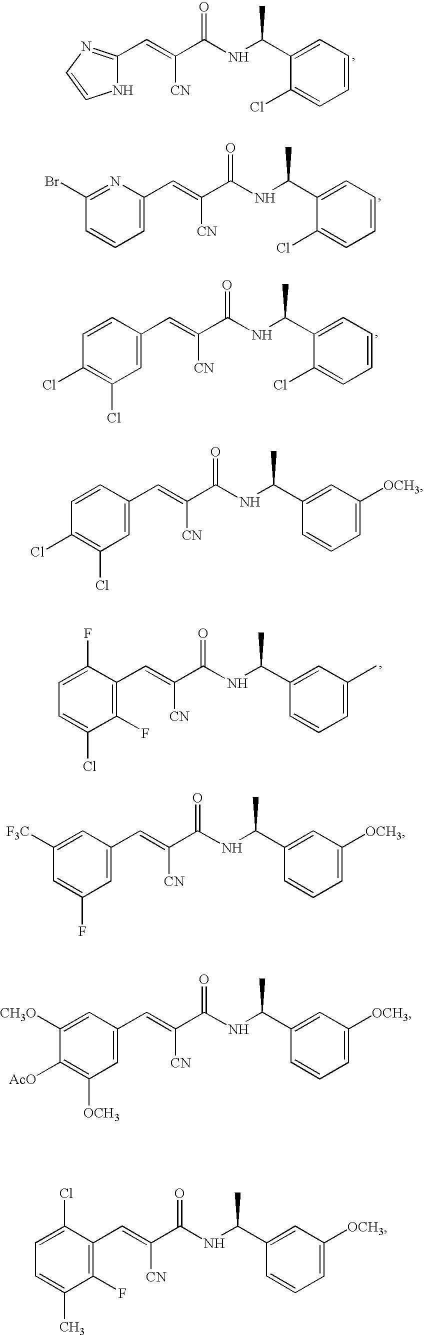

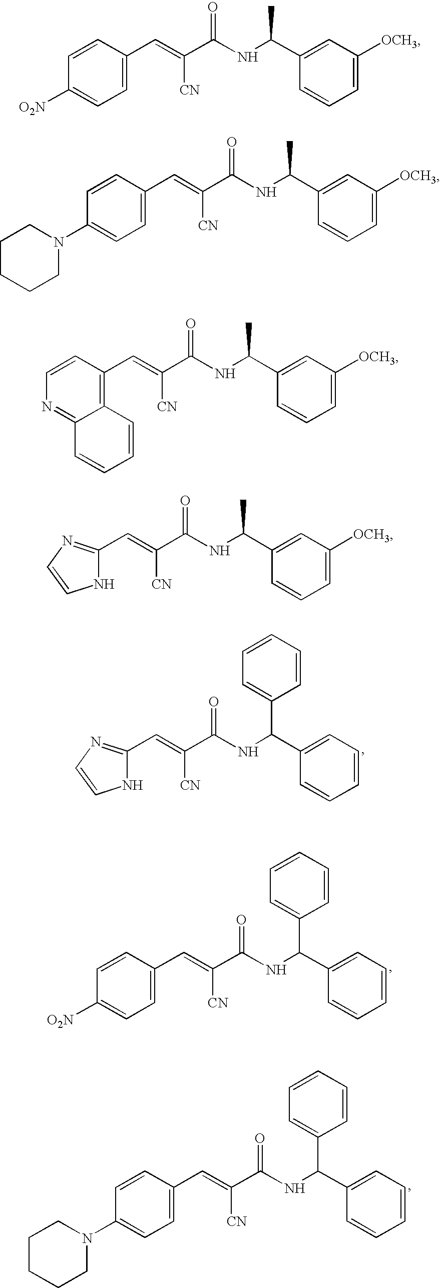

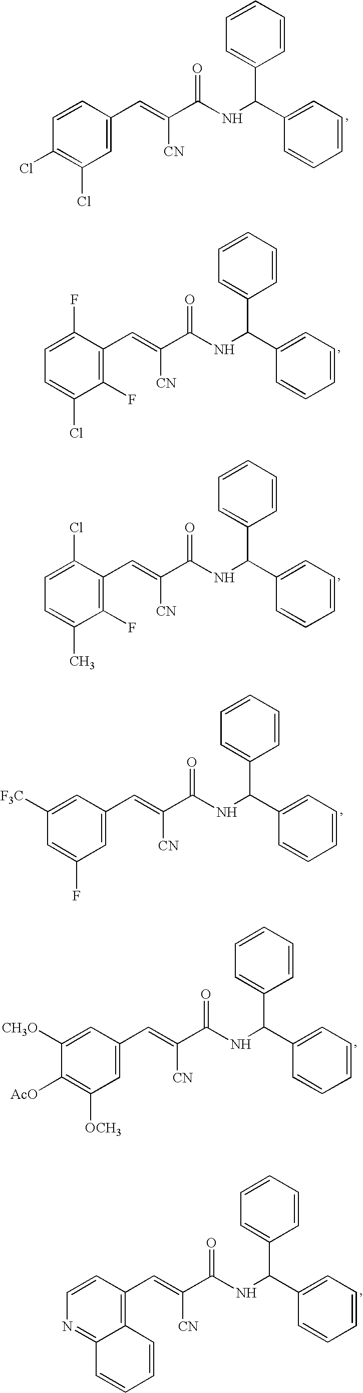

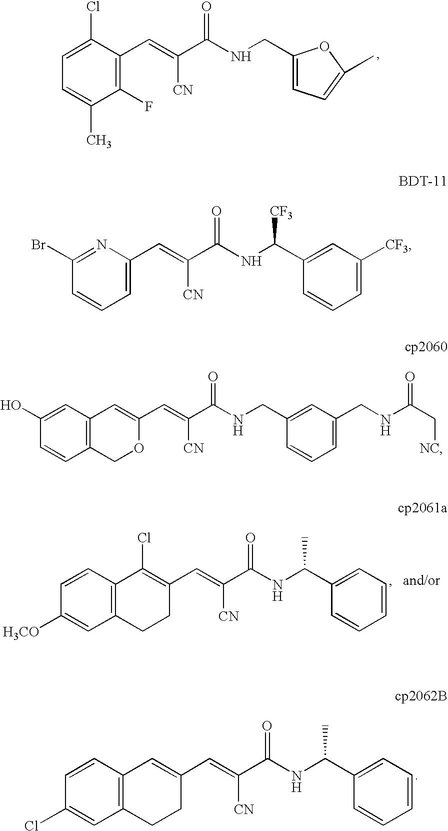

Solvent choices for synthetic methods described herein will be known to one of ordinary skill in the art. Solvent choices may depend, for example, on which one(s) will facilitate the solubilizing of all the reagents or, for example, which one(s) will best facilitate the desired reaction (particularly when the mechanism of the reaction is known). Solvents may include, for example, polar solvents and non-polar solvents. Solvents choices include, but are not limited to, tetrahydrofuran, dimethylformamide, dimethylsulfoxide, dioxane, methanol, ethanol, hexane, methylene chloride and acetonitrile. More than one solvent may be chosen for any particular reaction or purification procedure. Water may also be admixed into any solvent choice. Further, water, such as distilled water, may constitute the reaction medium instead of a solvent.

Persons of ordinary skill in the art will be familiar with methods of purifying compounds of the present invention. One of ordinary skill in the art will understand that compounds of the present invention can generally be purified at any step, including the purification of intermediates as well as purification of the final products. In preferred embodiments, purification is performed via silica gel column chromatography or HPLC.

As used throughout this application, the term “mammals” comprises humans. And the term “cell” refers to mammalian cells, including human cells.

The terms “interference,” “inhibiting,” “reducing,” or “prevention,” or any variation of these terms, when used in the claims and/or the specification includes any measurable decrease or complete inhibition to achieve a desired result.

It is contemplated that any embodiment discussed in this specification can be implemented with respect to any method or composition of the invention, and vice versa. Furthermore, compositions of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Assessment of Stat3 inhibition by Degrasyn: Degrasyn blocks Stat3 activation through reduction of soluble Jak2 protein levels.

Left: MM-1 cells were pretreated with Degrasyn (5 μM WP1130) for 2 h before incubation with IL-6 (10 ng/ml) for 15 min. Cell lysates were prepared and assessed for Stat3 activation (pY-Stat3) and protein levels (Stat3) by immunoblotting. Actin was used as a control for protein loading. Degrasyn blocked Stat3 activation.

Right: MM-1 cells were treated as described above and cell lysates were subjected to immunoprecipitation with anti-Jak2 (lanes 2-4) or control IgG (lane 1). Immune-complexes were resolved by SDS-PAGE, transferred to nitrocellulose and immunoblotted for tyrosine phosphorylated Jak2 (pY-Jak2; top) or Jak2 (bottom). Stat3 inhibition by Degrasyn is associated with loss of Jak2 protein recovery.

FIG. 2 Degrasyn is more effective that AG490 in suppressing Jak2/Stat3 signaling.

Left: MM-1 cells were pretreated with buffer alone (−), AG490 (50 μM) or Degrasyn (5 μM) for 2 h before incubation with IL-6 for 15 min. Cell lysates were prepared and Jak2 was immunoprecipitated and immunoblotted with anti-Jak2 (top). Cell lysates (50 μg) were immunoblotted for pY-Stat3, Stat3 or actin.

Right: Same as above, using Degrasyn alone.

Degrasyn reduces Stat3 activation more effectively than 10-fold greater concentrations of AG490 and is associated with a reduction of soluble Jak2 protein.

FIG. 3 Kinetics of Degrasyn mediated Jak2 down-regulation in MM-1 cells.

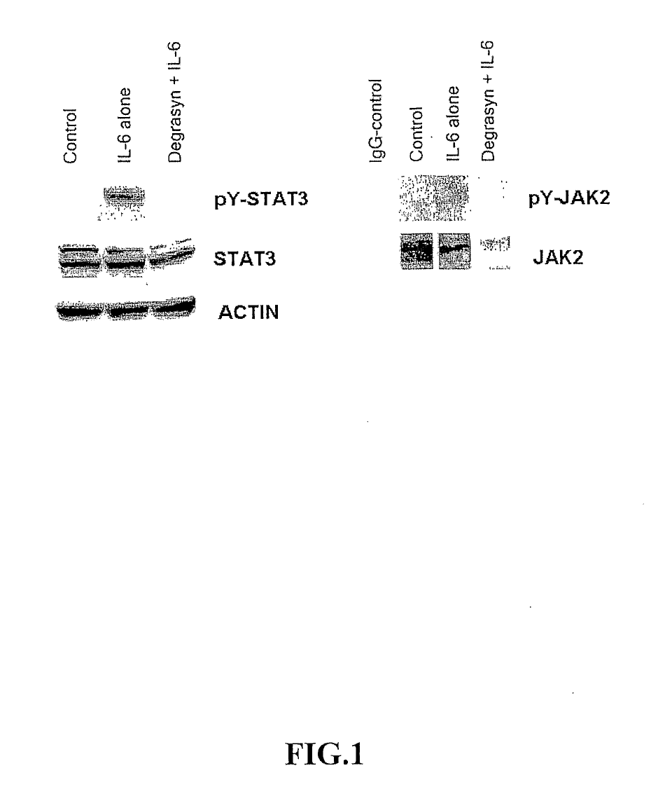

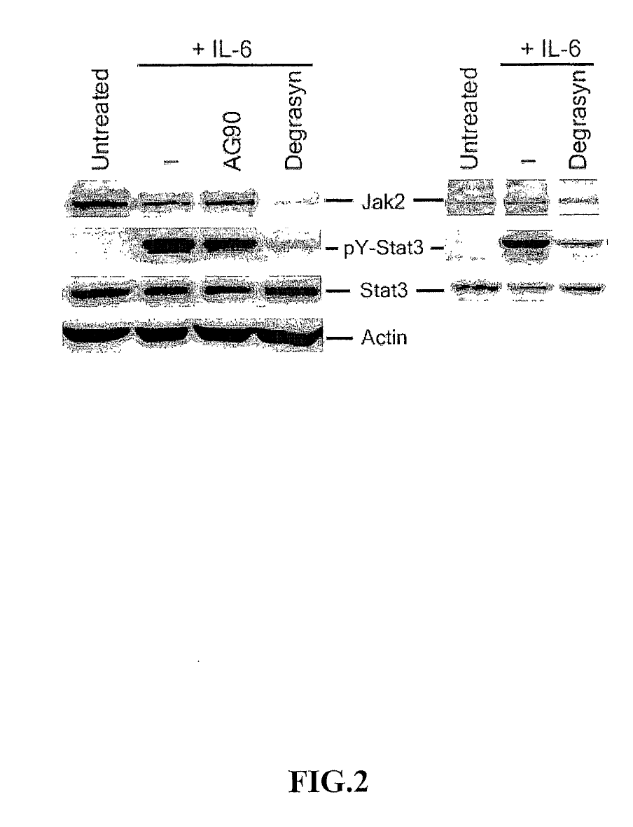

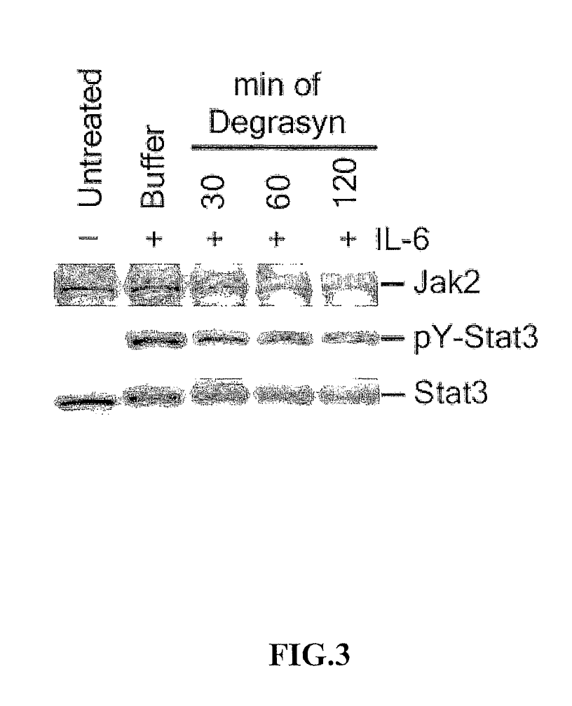

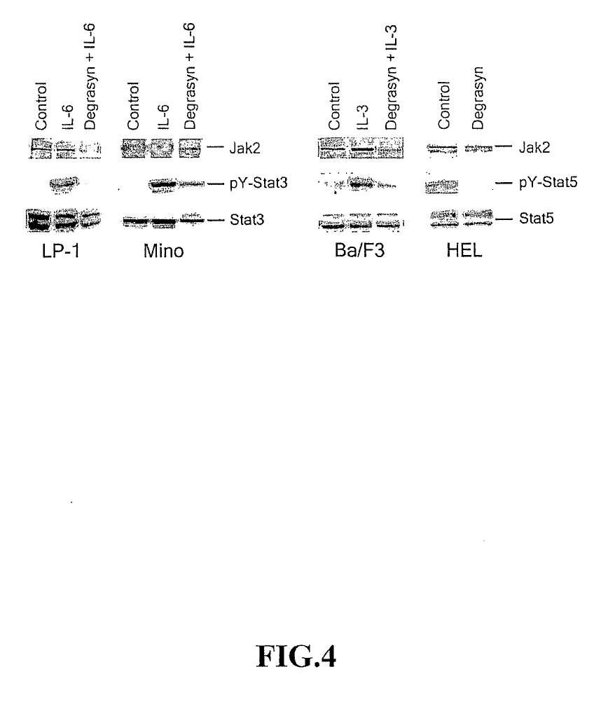

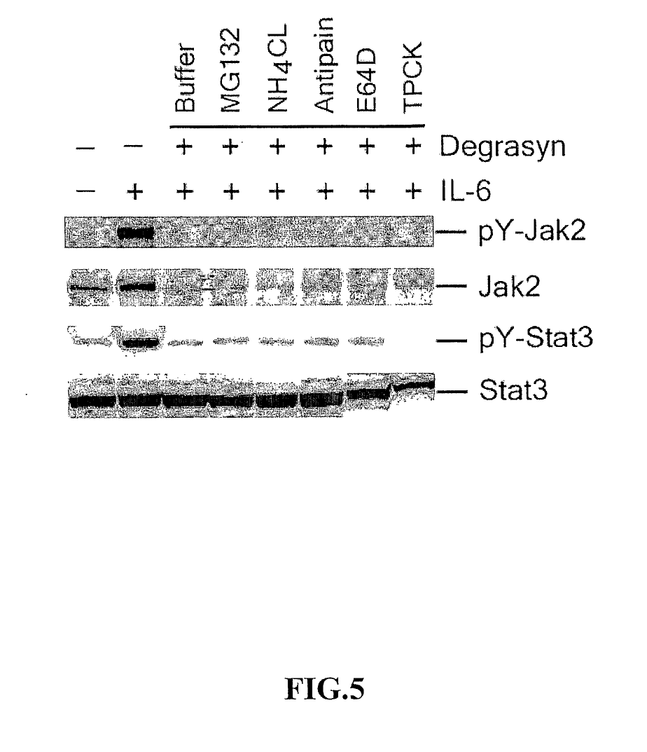

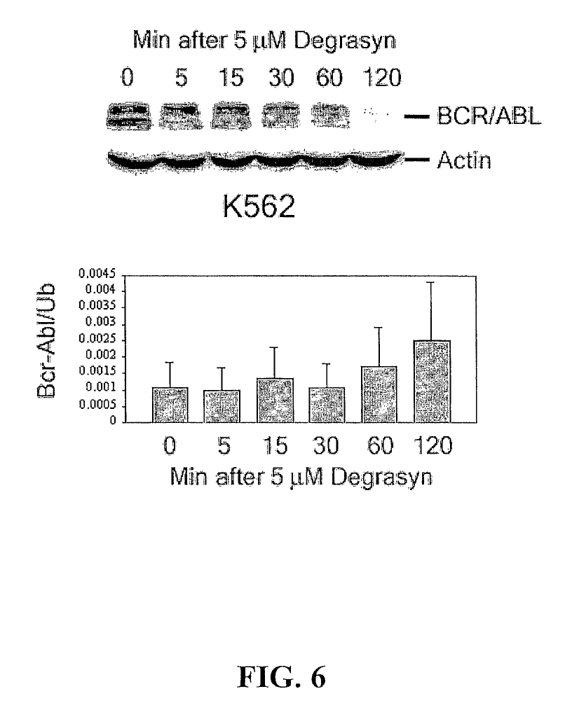

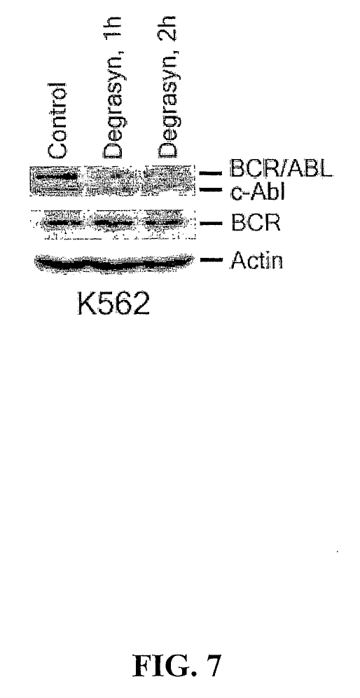

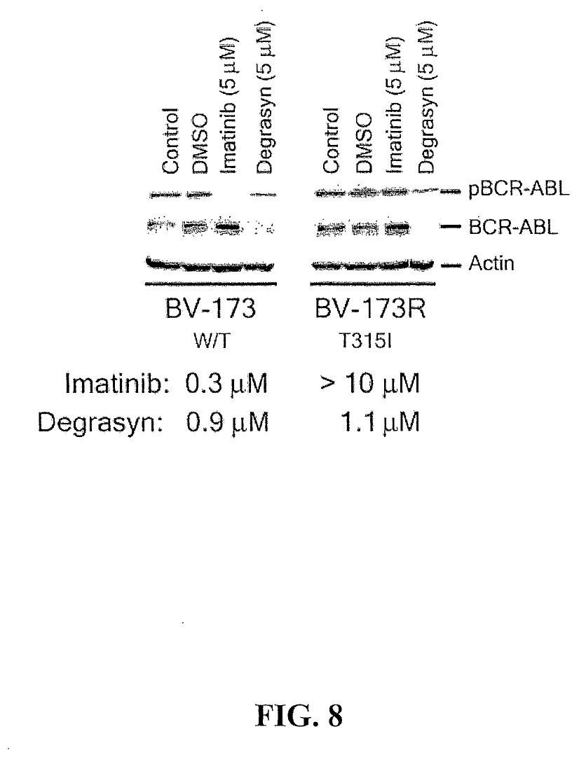

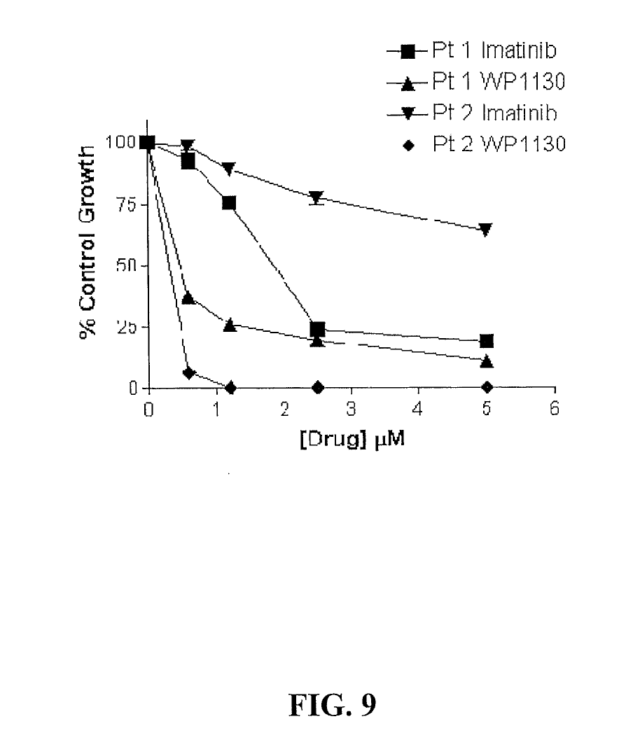

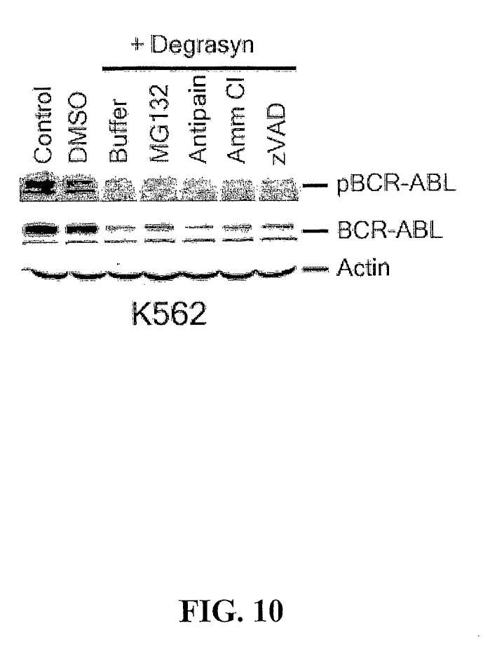

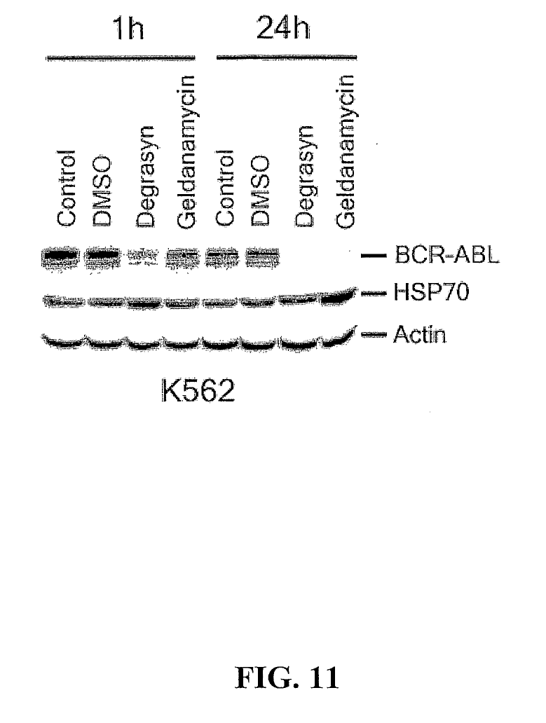

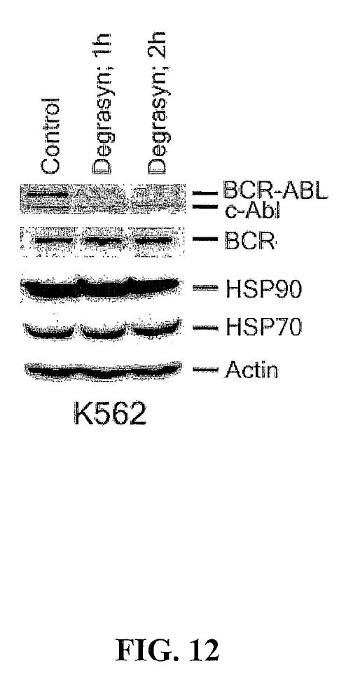

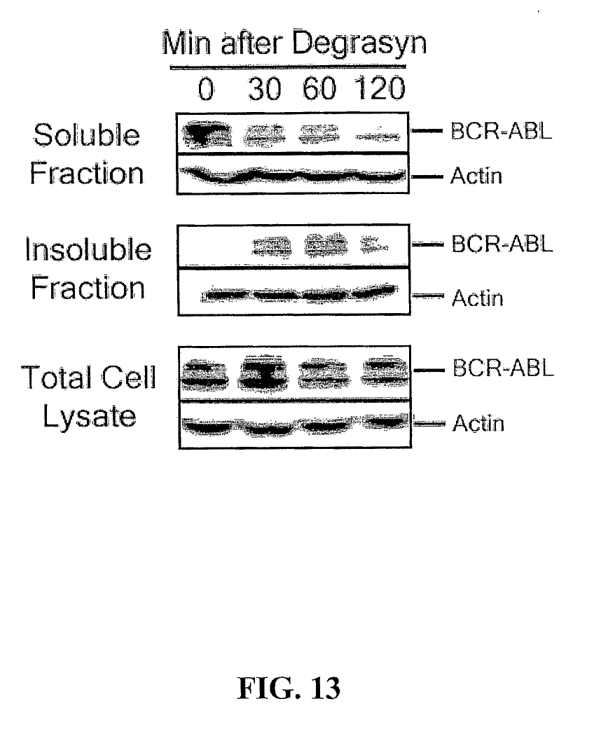

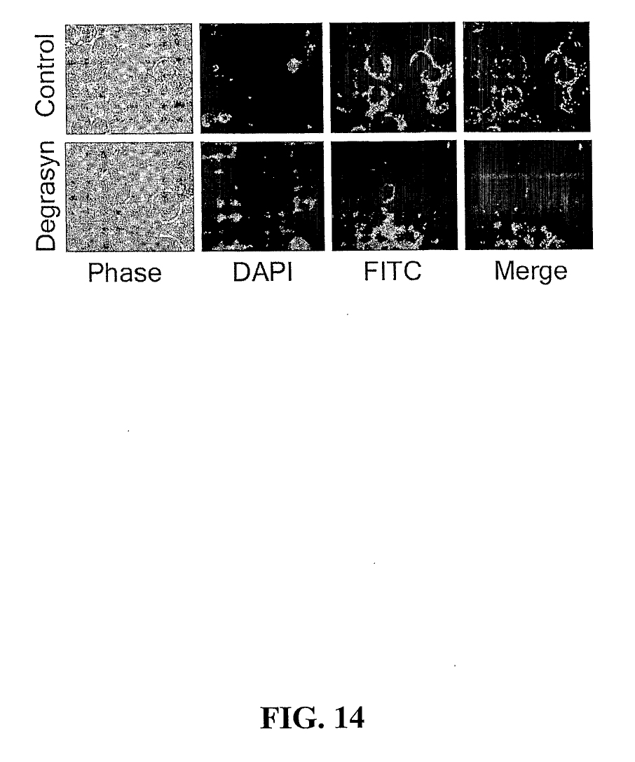

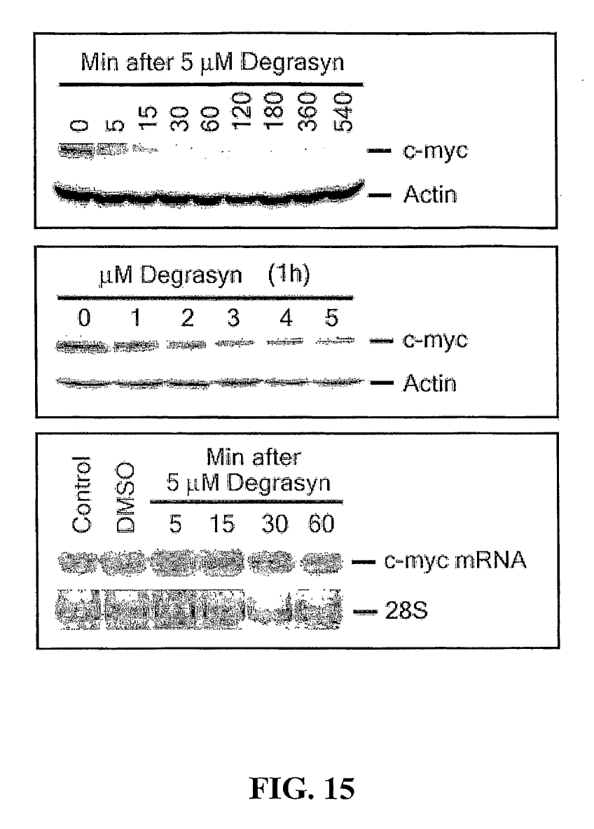

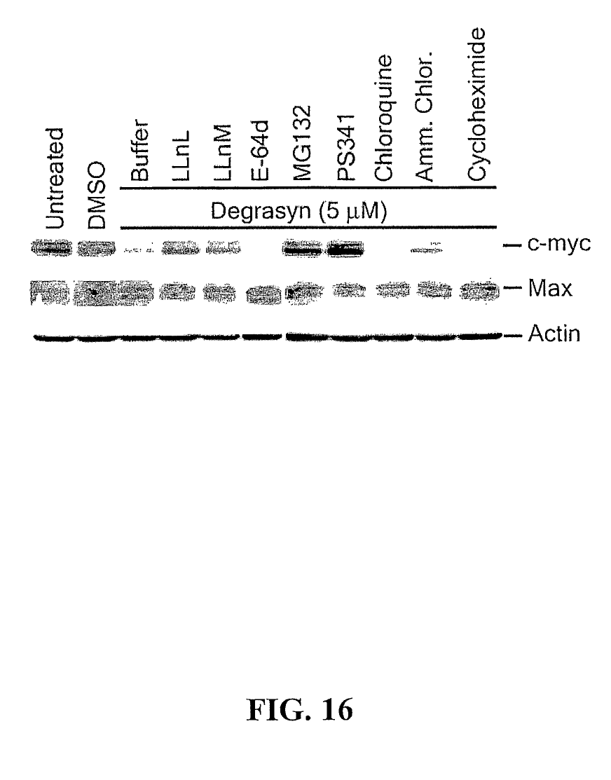

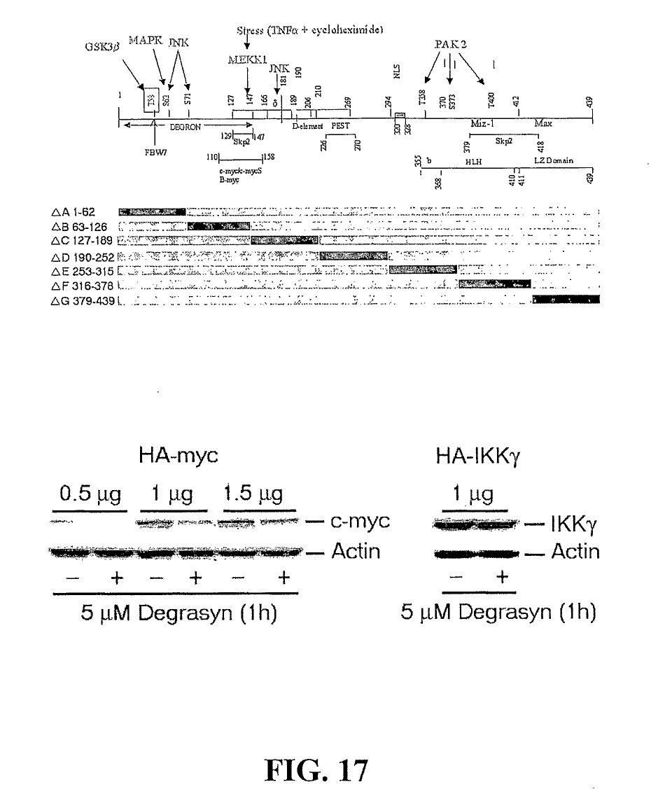

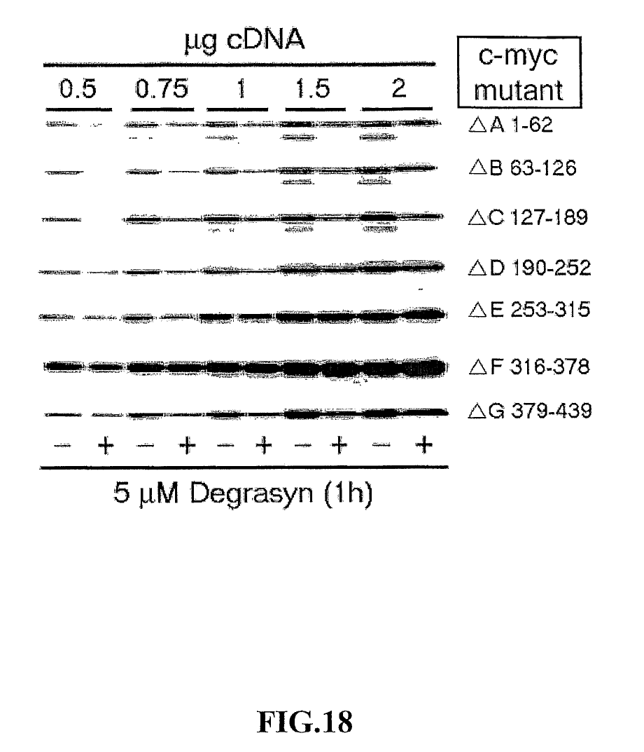

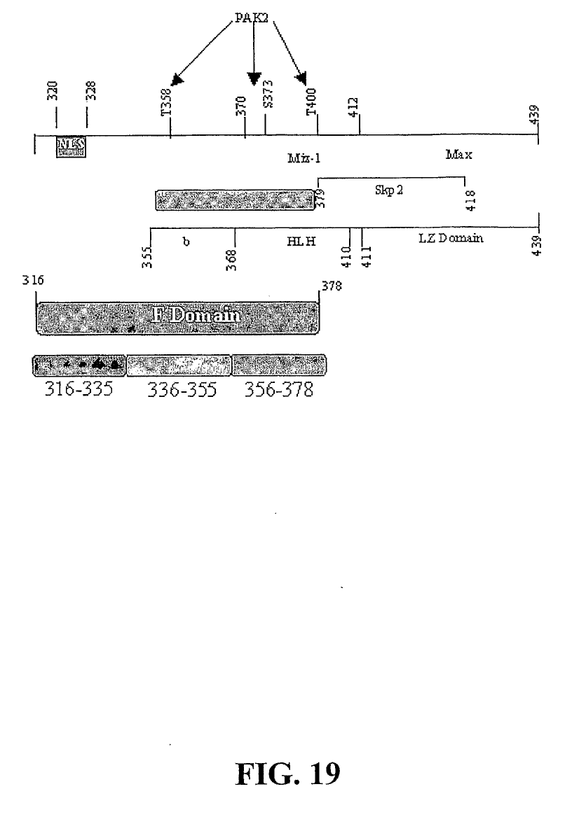

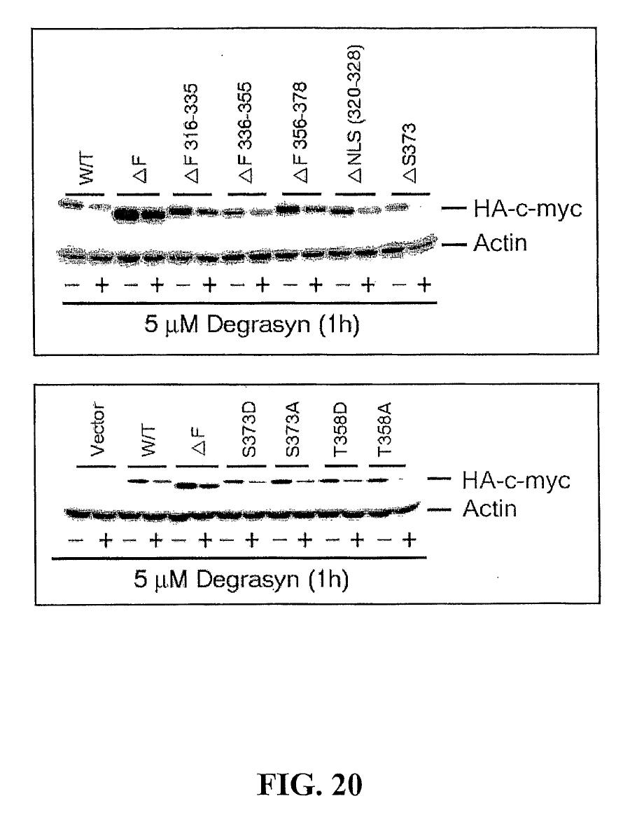

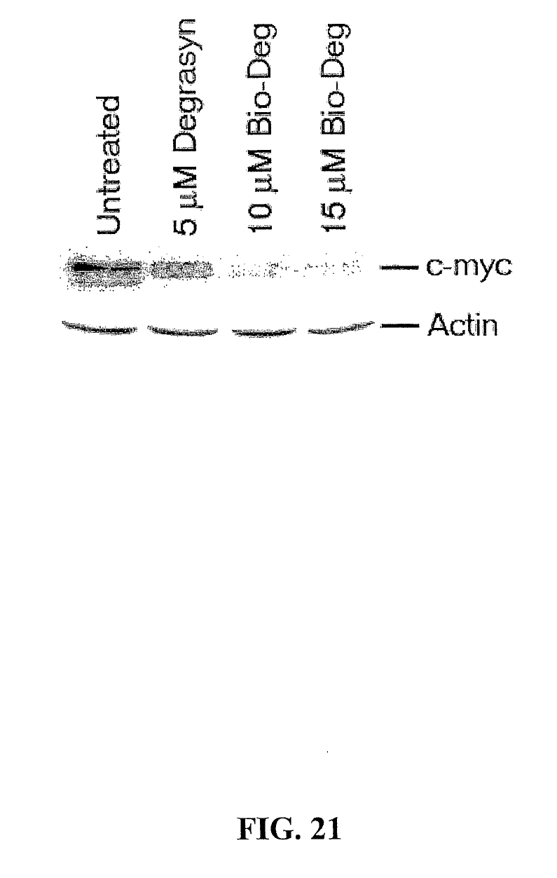

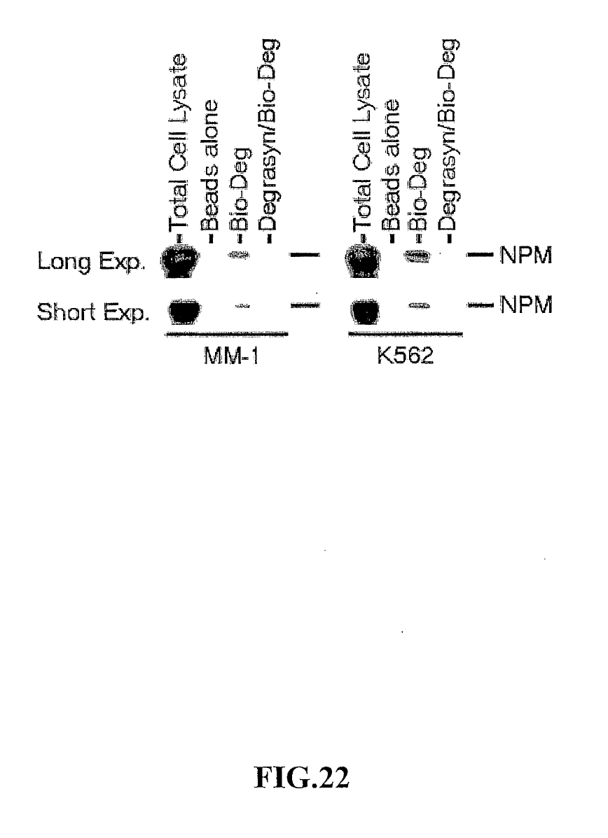

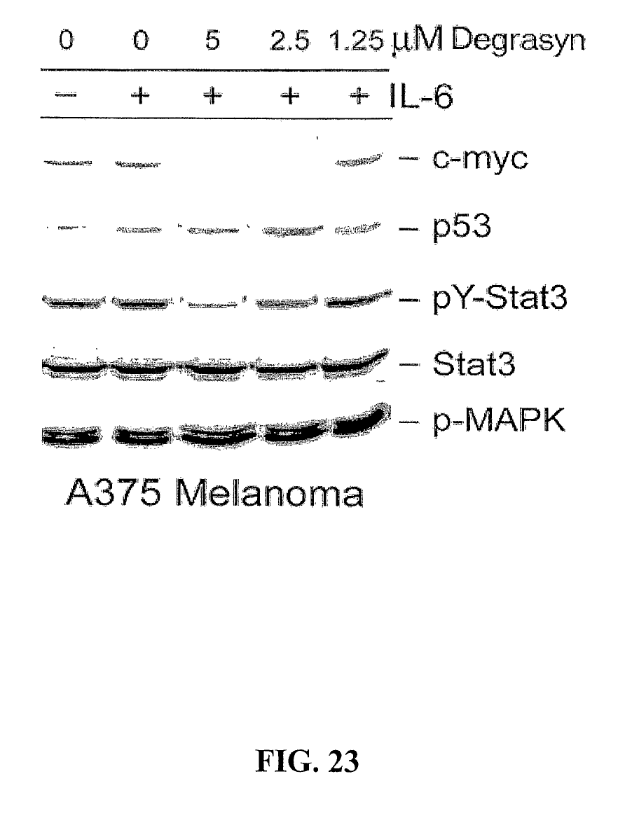

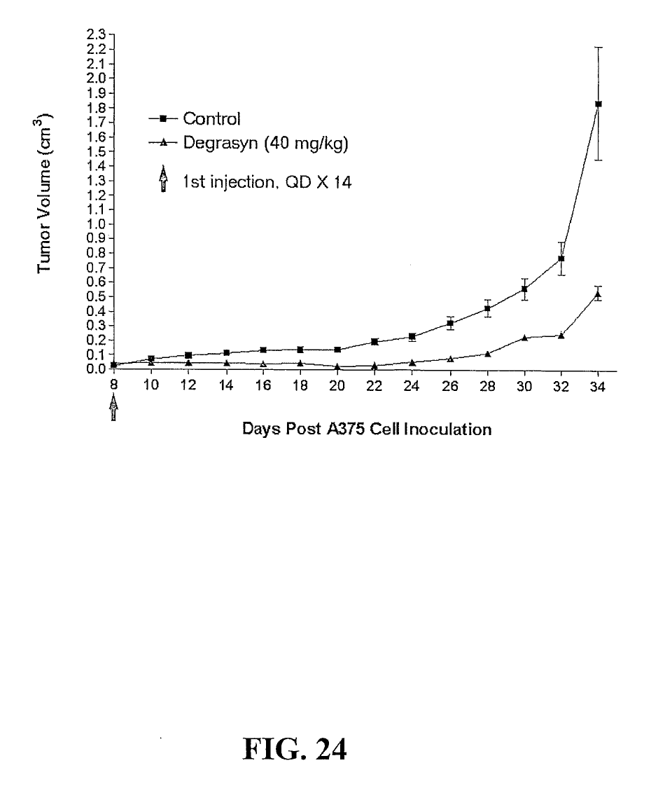

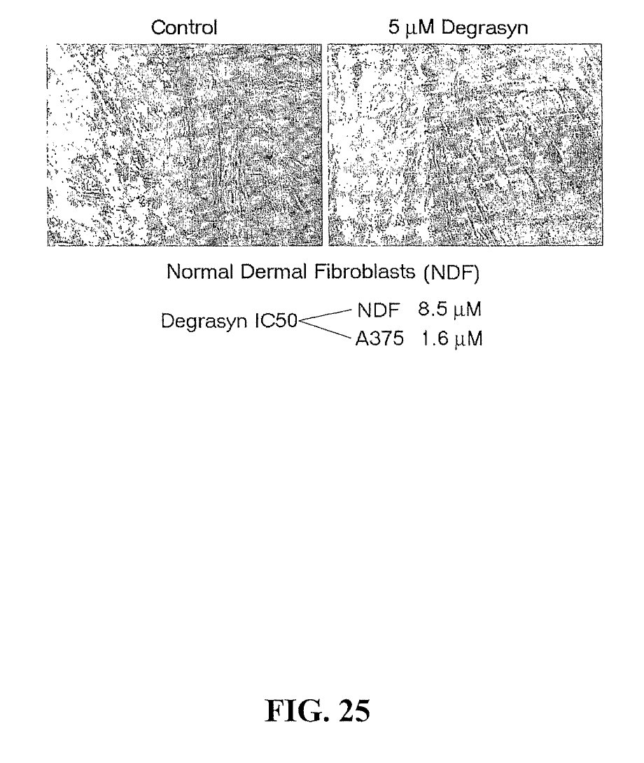

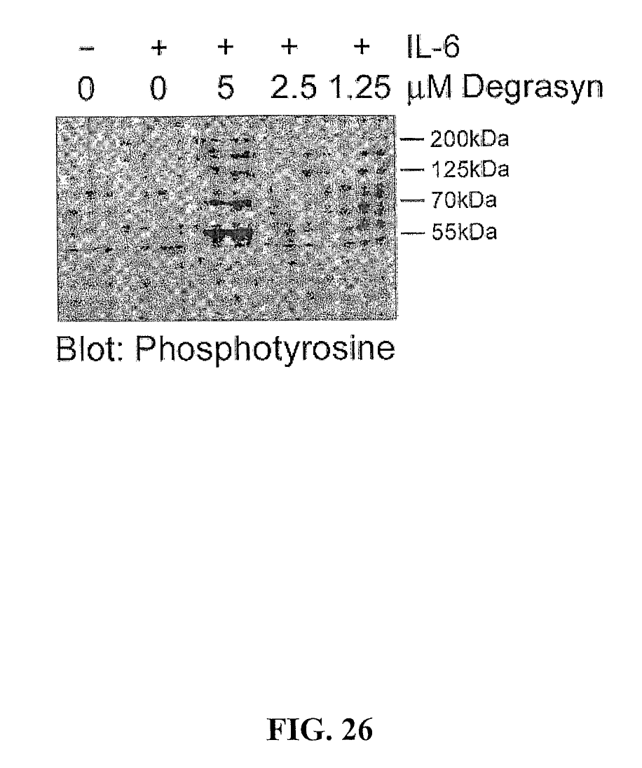

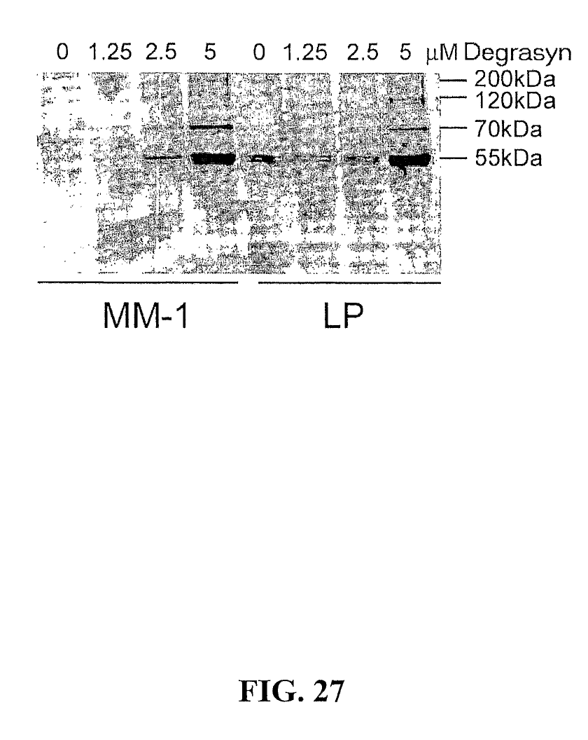

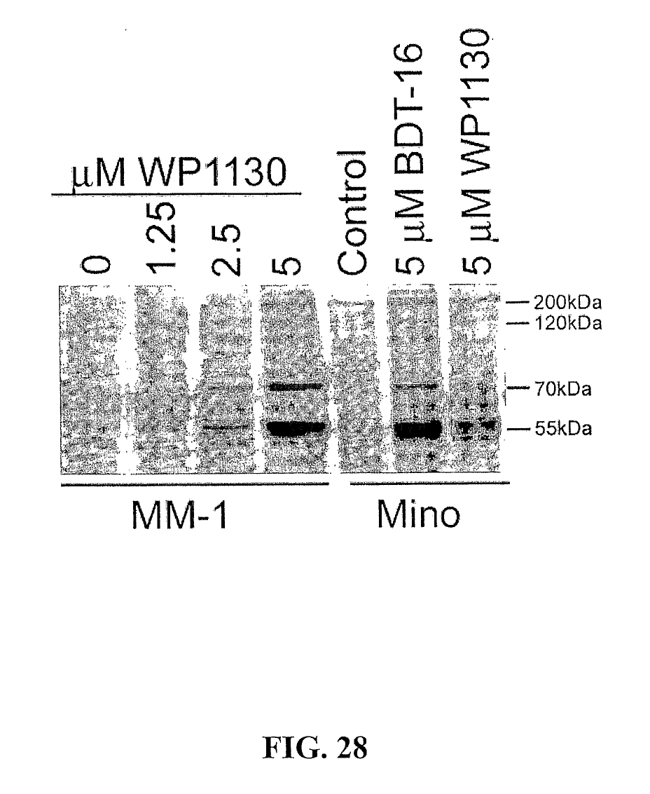

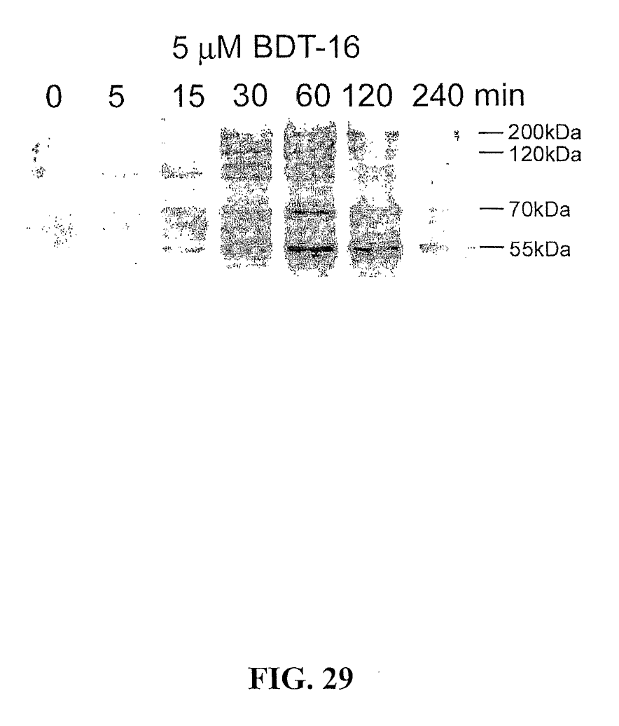

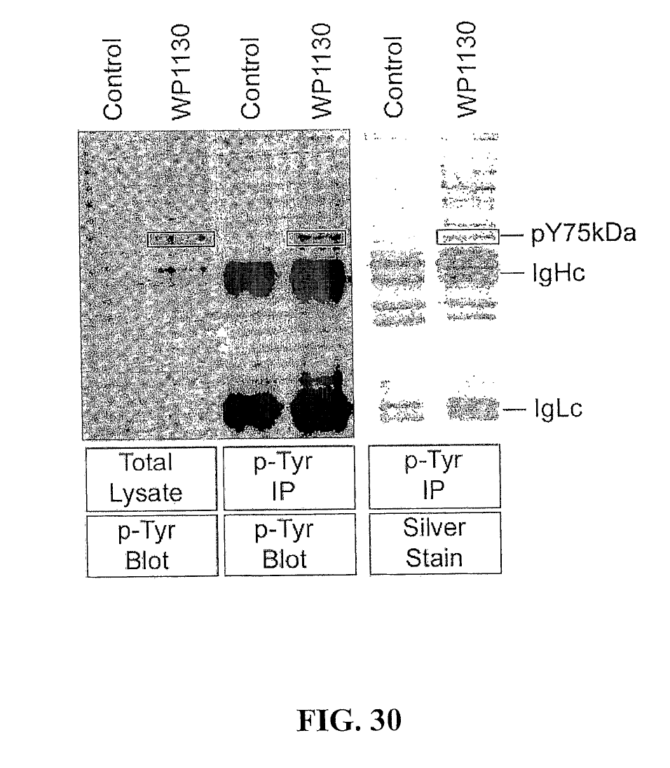

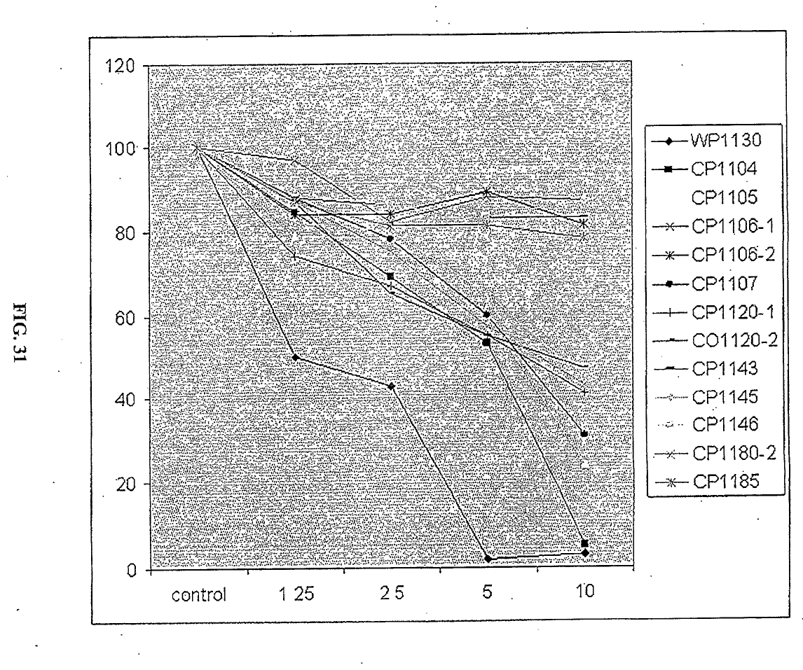

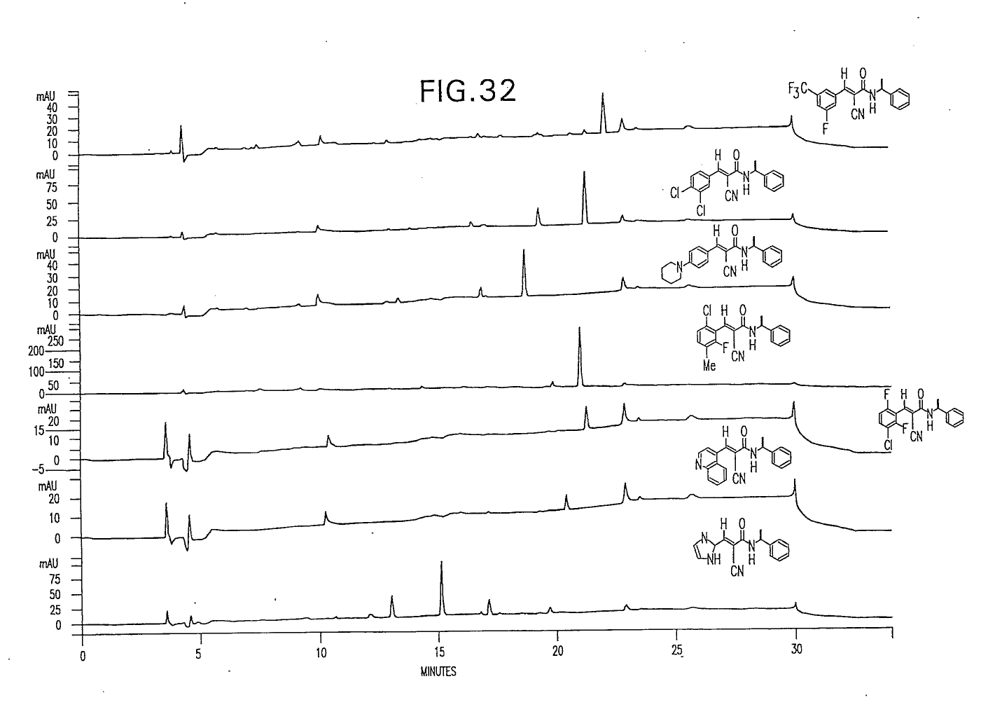

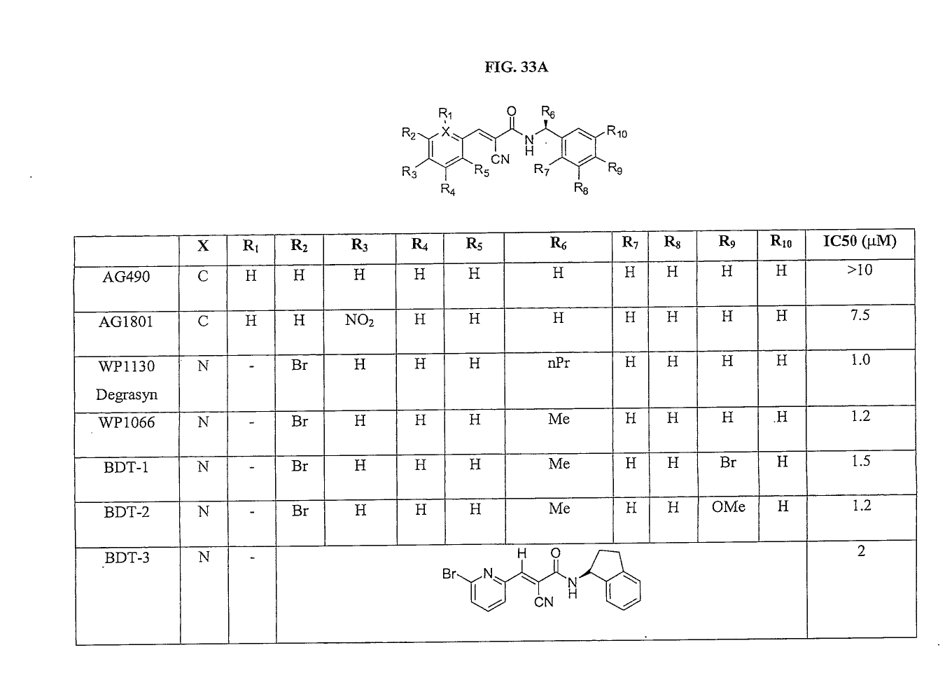

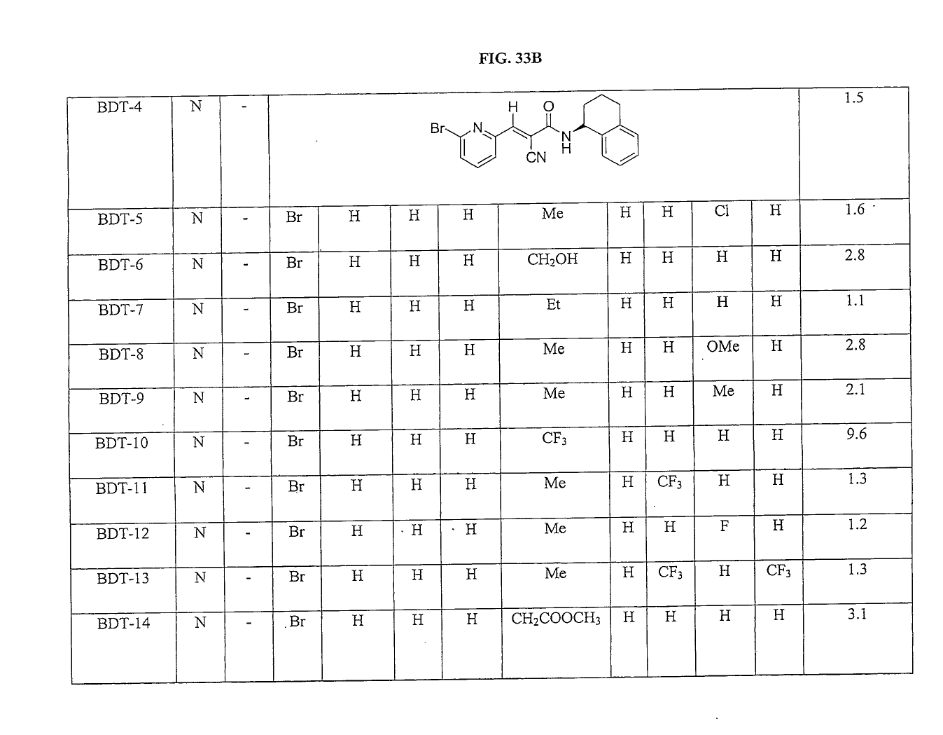

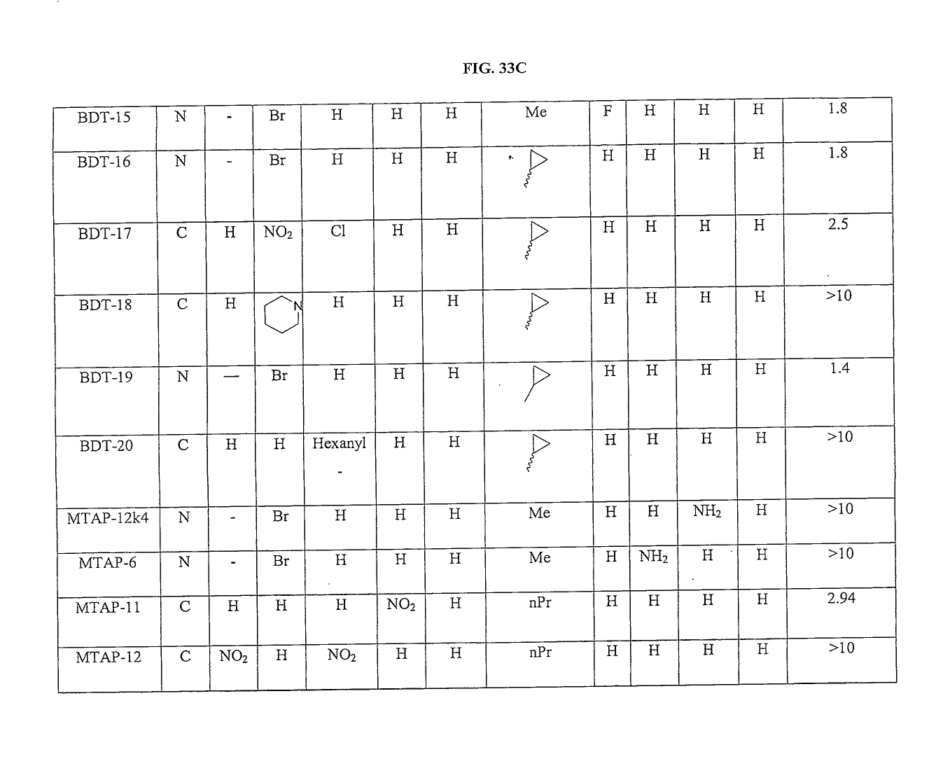

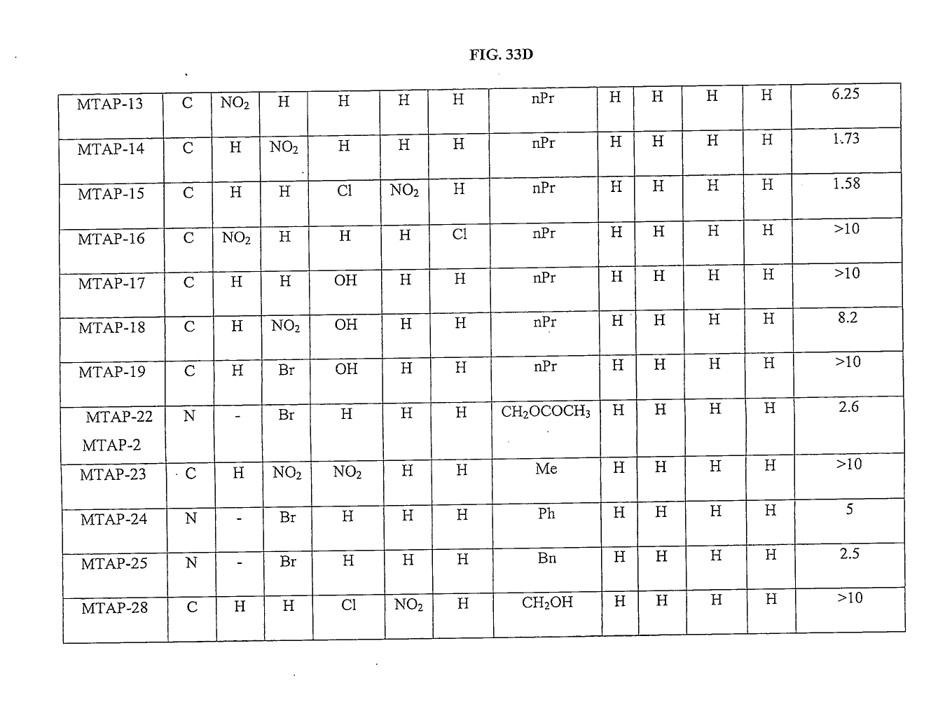

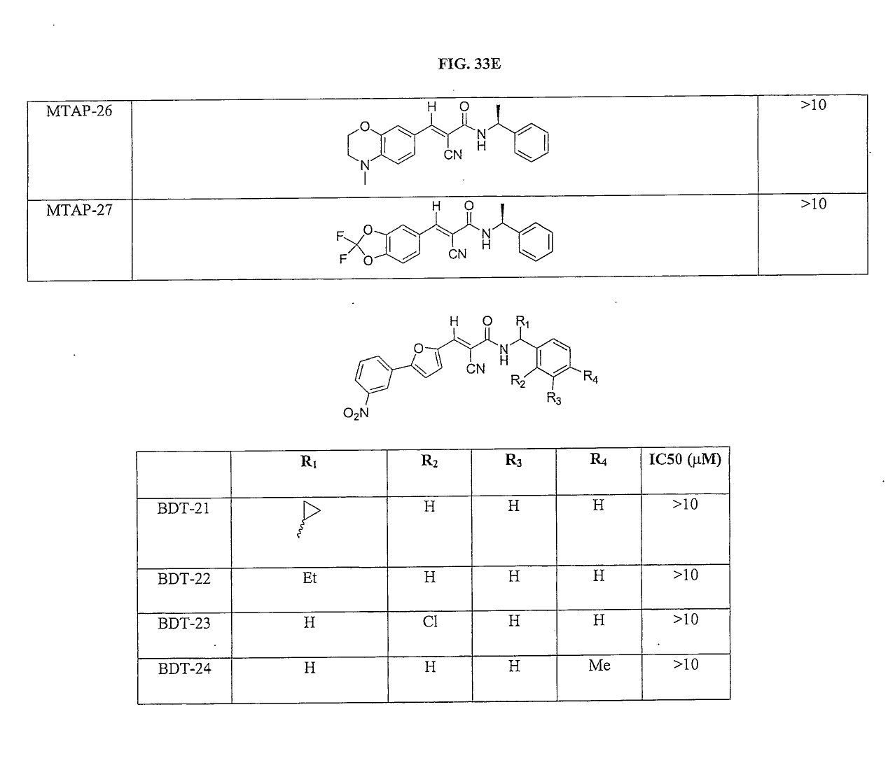

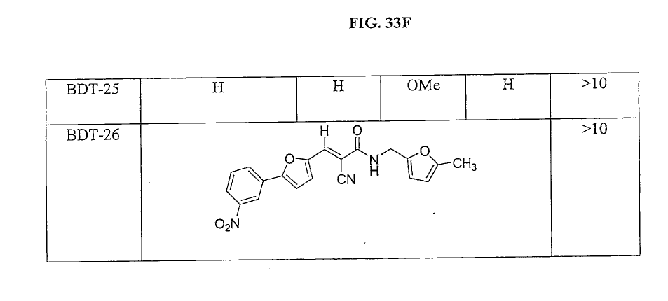

MM-1 cells were pretreated with Degrasyn (5 μM) for the interval noted before the addition of IL-6 (for 15 min). Jak2, pY-Stat3 and Stat3 were assessed as described in FIG. 1.

Loss of soluble Jak2 protein and inhibition of Stat3 activation is measurable after as little as 30 min of Degrasyn treatment.

FIG. 4 Degrasyn-mediated suppression of Stat3 and 5 activation in human and murine cells correlates with loss of soluble Jak2 protein expression.

B-cell malignancies from multiple origins (LP; non-Hodgkin's lymphoma, Mino; Mantle cell lymphoma, Ba/F3; murine IL-3 dependent proB-cell lymphoma, HEL; human erythroid leukemia) were treated with IL-3 or IL-6 for 15 min or pretreated with Degrasyn (5 μM, 2 h) before the addition of cytokine. Jak2, pY-Stat3, Stat3, pY-Stat5 and Stat5 protein levels were analyzed as described in FIG. 1.

Degrasyn reduced the level of soluble Jak2 in these cells, resulting in loss of cytokine-mediated Stat3 and Stat5 activation.

FIG. 5 Protease inhibitors do not block Degrasyn activity against Jak2/Stat3.

MM-1 cells pre-incubated with protease inhibitors (MG132; 40 μM, ammonium chloride; 2.5 mM, antipain; 10 μM, E64D; 100 μM, TPCK; 10 μM) followed by the addition of 5 μM Degrasyn for an additional 2 h. Cells were then treated with IL-6 for 15 min (as indicated) before analysis of pY-Jak2, Jak2, pY-Stat3 and Stat3 protein levels as described in FIG. 1. Protease inhibitors did not block Degrasyn-mediated loss of soluble Jak2 protein in MM-1 cells.

FIG. 6 Degrasyn reduces the level of the Bcr-Abl oncoprotein without reducing Bcr-Abl mRNA levels in CML cells.

Top: The BCR-ABL expressing CML cell line (K562) was incubated with 5 μM Degrasyn for the interval noted before cells were lysed and protein extracts were analyzed for BCR-ABL protein (using anti-Abl) levels by immunoblotting. The blot was stripped and reprobed for actin as a protein loading control.

Bottom: RNA from cells treated as described above was isolated (TRIzol Reagent) and used as a template to generate cDNA. This material was then used to prime a real-time PCR reaction for bcr-abl and ubiquitin (Ub) transcripts. The values are reported in terms of the ratio of bcr-abl to Ub. Degrasyn reduced the soluble BCR-ABL protein level without suppressing bcr-abl mRNA expression.

These results suggest a post-transcriptional mechanism of Degrasyn action against BCR-ABL.

FIG. 7 Degrasyn reduces soluble BCR-ABL protein without effects on BCR and c-Abl.

K562 cells were treated with Degrasyn (2 h, 5 μM) before cells were lysates were prepared and analyzed for BCR-ABL, c-Abl and BCR protein levels. Actin was probed on the stripped blot to control for protein loading.

Degrasyn reduced BCR-ABL protein levels but had minimal effects on c-Abl and BCR.

FIG. 8 Degrasyn reduces BCR-ABL protein in both wild-type and mutant BCR-ABL expressing cells, resulting in a reduction in pBCR-ABL levels.

Top: BV-173, a lymphoid CML cell line expressing w/t BCR-ABL and the BV-173R cell line variant expressing a kinase inhibitor resistant form or BCR-ABL (T315I) were treated with 5 mM imatinib or Degrasyn for 1 h before cell extracts were probed for activated BCR-ABL (pBCR-ABL by pY immunoblotting) or BCR-ABL protein levels. Actin was probed as protein loading control.

Imatinib reduced BCR-ABL activation without effecting BCR-ABL protein levels in BV-173. Imatinib was inactive in the BV-173R cell type that expresses the T315I mutant kinase. Degrasyn reduced BCR-ABL protein levels in both w/t and mutant BCR-ABL expressing cells, resulting in a reduction in pBCR-ABL levels.

Bottom: The IC50 values for both cell types were calculated based on the results of an MTT assay after 72 h of drug incubation.

Imatinib was ineffective in BV-173R cells while Degrasyn was equally active against both cell types.

FIG. 9 Degrasyn reduces the clonal outgrowth of CML cells from imatinib resistant patients expressing the T315I form of BCR-ABL.

CML cells from 2 patients that failed imatinib therapy were characterized and shown to express the T315I mutant BCR-ABL. Cells were incubated with the indicated concentration of imatinib or Degrasyn and plated to determine the impact of drug exposure on the outgrowth of leukemic cells as described in the methods (above).

Degrasyn was more effective than imatinib in suppressing colony formation in both patients.

FIG. 10 Protease inhibitors do not block Degrasyn-mediated BCR-ABL downregulation.

K562 cells were pretreated with protease inhibitors as described in FIG. 5 (zVAD used at 50 μM) and Degrasyn (5 mM, 2 h) before analysis of BCR-ABL phosphorylation and protein levels by immunoblotting.

As described for Jak2 (FIG. 6), protease inhibitors did not block Degrasyn-mediated loss of BCR-ABL protein.

FIG. 11 Degrasyn-mediated loss of BCR-ABL protein is distinct from geldanamycin and does not lead to induction of HSP70.

K562 cells were treated with nothing (Control), vehicle (DMSO; 0.1%), 5 μM Degrasyn or 5 μM geldanamycin for the interval indicated. Cell lysates were prepared and BCR-ABL, HSP70 and Actin were subjected to immunoblotting.

Degrasyn rapidly reduced recovery of BCR-ABL and did not increase HSP70. These activities were distinct in geldanamycin treated cells. These results suggest that BCR-ABL protein expression is regulated by different mechanisms in Degrasyn- and Geldanamycin-treated cells.

FIG. 12 Degrasyn-mediates downregulation of BCR-ABL oncoprotein through a distinct mechanism.

K562 cells were treated with 5 mM Degrasyn for 1 to 2 h before equal protein cell extracts were immunoblotted for BCR-ABL, c-Abl, BCR, HSP90, HSP70 and actin.

The results demonstrate that BCR-ABL (which is a cytoplasmic protein), but not c-Abl or BCR (which are nuclear proteins), is down-regulated following Degrasyn incubation. BCR-ABL down-regulation occurs in the absence of changes in HSP70 or HSP90, suggesting that loss of BCR-ABL protein occurs in the absence of an effect on these heat shock proteins.

FIG. 13 Differential subcellular partitioning of BCR-ABL following Degrasyn incubation.

K562 cells were treated with Degrasyn (5 μM) for the interval indicated before cell lysates were prepared (Soluble Fraction; prepared in buffer containing 1% NP-40, 0.5% Na-deoxycholate, 0.1% Na-dodecyl sulfate). The insoluble material from this lysate was collected by centrifugation (12,000×g, 15 min), resuspended in SDS sample buffer and sonicated (Insoluble Fraction). Cells treated in the same manner were directly lysed in the presence of SDS-sample buffer followed by sonication (Total Cell Lysate). Equal protein aliquots were subjected to immunoblotting for BCR-ABL and actin.

The results suggest that Degrasyn mediates transfer of BCR-ABL from a soluble fraction (cytoplasmic) to an insoluble compartment. Degrasyn did not reduce total cellular BCR-ABL content but altered its subcellular distribution.

FIG. 14 Degrasyn alters cellular distribution of BCR-ABL.

K562 cells were treated as described in FIG. 13 and pelleted onto microscope slides by CytoSpin apparatus. Cells were fixed and stained with DAPI to detect the cell nucleus (DAPI stains DNA), and incubated with anti-Abl followed by FITC labeled secondary antibody (to detect BCR-ABL). Slides with fixed and stained cells were subjected to deconvoluting microscopy and photographed with different filters to determine the partitioning of BCR-ABL following treatment. The DAPI and FITC images were merged to determine the extent of nuclear/cytoplasmic transfer after treatment.

Degrasyn reduced cytoplasmic staining of BCR-ABL.

FIG. 15 Degrasyn causes rapid reduction of c-myc protein in MM-1 cells.

Top: MM-1 cells were incubated with 5 μM Degrasyn for the interval noted before cell lysates were prepared and subjected to immunoblotting for c-myc and actin as a protein loading control. Degrasyn caused a rapid reduction of c-myc protein.

Middle: MM-1 cells were treated with the indicated concentration of Degrasyn for 1 h before lysates were immunoblotted for c-myc and actin. Degrasyn caused a concentration dependent reduction in c-myc protein levels.

Bottom: MM-1 cells were treated as indicated before extraction of total RNA (TRIzol). RNA was resolved by agarose electrophoresis, transferred to a membrane and hybridized with a c-myc probe to determine the level of c-myc RNA in each sample. The ethidium bromide stained gel was photographed and 28S RNA content was detected as a measure of RNA content in each lane.

The results suggest that Degrasyn reduces c-myc protein content without effecting c-myc gene expression.

FIG. 16 Degrasyn reduces c-myc protein levels through a proteosome-dependent process.

MM-1 cells were pretreated with protease/proteosome inhibitors (LLnL; LLnM; E-64d; MG132; PS341; Chloroquine; Ammonium Chloride; Cycloheximide) for 2 h before the addition of 5 μM Degrasyn or vehicle alone (DMSO as indicated) for an additional 2 h. Cell lysates were prepared and equal protein aliquots were subjected to immunoblotting for c-myc, Max (a c-myc binding partner) or actin as a protein loading control. The results demonstrate that proteosome inhibitors (MG132, PS341) block Degrasyn-mediated c-myc protein reduction.

FIG. 17 A map of critical elements on c-myc involved in its destruction following Degrasyn.

Top: A c-myc protein map of critical elements involved in its stabilization and destruction. Shown are the amino acid sites previously shown to be involved in phosphorylation (GSK3b, MAPK, JNK, MEKK1, PAK2) that direct c-myc destruction and presentation to the proteosome are shown above the linear representation of the c-myc protein (labeled at the termini as 1 and 439). The domains involved in accessory protein binding, are also depicted before the linear representation of c-myc.

Middle: The yellow/red diagram depicts domains on c-myc that were deleted by gene mutation (red region) from the parent c-myc protein (deletions were ˜62 amino acid sequences that encode critical determinant regions important for c-myc stability. The orange box depicts the nuclear localization signal (NLS) of c-myc. These mutants were expressed and examined for their effect on Degrasyn-mediated degradation in FIG. 18.

Bottom: Expression vectors for HA-tagged c-myc or HA-tagged IKKγ (tagged at the N-terminus) were transfected (at the DNA content indicated) into HeLa cells as described in the methods section above. Twenty-four h later, cells were treated with Degrasyn for 1 h before harvesting cells, extracting protein and subjecting equal protein lysates to immunoblotting for c-myc (left) or IKKγ (right) using an HA-directed antibody. Actin was immunoblotted as a control for protein loading. Degrasyn reduced HA-tagged c-myc but did not affect HA-tagged IKKγ. These cells and transfection system were used to map the domains important for Degrasyn mediated destabilization by deletion analysis (FIG. 20).

FIG. 18 Deletion of amino acids 316-378 on c-myc reduces its destabilization and destruction by Degrasyn.

HeLa cells were transfected with the HA-tagged c-myc deletion construct indicated (at the indicated DNA content) for 24 h before incubation with Degrasyn. Cell lysates were prepared and immunoblotted for HA-c-myc with anti-HA.

Deletion of the Δ316-378 domain of c-myc reduced its sensitivity to destruction by endogenous and Degrasyn-stimulated mechanisms.

FIG. 19 Fine mapping of the ΔF c-myc for sensitivity to Degrasyn.

The ΔF domain of c-myc was subjected to further deletion (by mutagenesis) to determine the smallest region necessary to reduce Degrasyn-mediated c-myc destruction. Constructs included those that delete ˜20 amino acids within the ΔF of c-myc, the NLS (amino acids 320-328) and critical serine (S373) or threonine (T358) residues in this region.

FIG. 20 Two regions within the ΔF domain of c-myc are involved in its destabilization by Degrasyn.

HeLa cells were transfected with the indicated deletion construct of HA-c-myc (0.75 μg DNA) for 24 h before treatment with Degrasyn for 1 h. Cell lysates were prepared and immunoblotted for HA-c-myc using anti-HA. The membrane was also probed with anti-actin as a protein loading control. Deletion of the NLS, 5373 site or modifications at critical phosphorylation sites, did not affect Degrasyn-mediated down-regulation of c-myc. Deletion of amino acids 316-335 and 356-378 of c-myc resulted in reduced sensitivity to Degrasyn-mediated c-myc destruction. These results suggest that two regions within the ΔF region form a critical determinant on c-myc that regulates Degrasyn sensitivity. This region has not previously been reported to regulate c-myc stability in tumor cells.

FIG. 21 Biotinylated Degrasyn retains its c-myc modulatory activity.

Degrasyn was synthesized with a biotinylated side-chain (Bio-Degrasyn; MTAP-biotin, vide infra) and compared to Degrasyn for its c-myc down-regulatory and anti-tumor activity. Biotinylation of Degrasyn reduced its anti-tumor activity in MM-1 and K562 cells by ˜2-fold.

Degrasyn IC50 for MM-1/K562 cells=1.4/2.8 μM.

Bio-Degrasyn IC50 for MM-1/K562 cells=3.8/5.6 μM.

Pictured: MM-1 cells were treated with biotinylated Degrasyn (Bio-Deg) or Degrasyn (at the indicated concentration) for 1 h before analyzing effects on c-myc as described in FIG. 15.

Treatment with Degrasyn and Bio-Deg reduced c-myc protein levels in MM-1 cells, suggesting that both the anti-tumor and c-myc modulatory activity were retained in biotinylated Degrasyn.

FIG. 22 Identification of Degrasyn interactive proteins in MM-1 and K562 cells.

Biotinylated-Degrasyn (10 μM) was incubated with lysates from MM-1 cells (10 mg total protein) and interactive proteins were assessed by affinity recovery using Streptavidin-conjugated beads to bind biotinylated-Degrasyn. Free Degrasyn (10 μM) was incubated with biotinylated-Degrasyn in a parallel experiment to assist in identifying specific protein:Degrasyn interactions. Affinity complexes were washed and Streptavidin-Bio-Deg interaction proteins were assessed by disrupting protein complexes with SDS-sample buffer and resolving proteins by SDS-PAGE. The resolved proteins were detected by silver-staining the gel. Protein bands present in the biotinylated-Degrasyn sample that were absent in samples co-incubated with free Degrasyn were identified and cut from the gel. A 38 kDa protein band was prominent in this assessment and was subjected to in gel trypsinization and identification of HPLC resolved tryptic peptides by tandem mass spectroscopy analysis (David Hawke, Department of Molecular Pathology, M.D. Anderson Cancer Center). This analysis identified nucleophosmin (NPM) as a Degrasyn interactive protein.

To further assess the interaction between NPM and Degrasyn, biotinylated-Degrasyn/streptavidin beads were incubated with MM-1 (left) or K562 (right) cell lysates. In parallel, lysates were co-incubated with free Degrasyn to determine the specificity of the interaction with NPM. After 2 h incubation, beads were washed and interactive proteins were released by heating in SDS-sample buffer. The proteins were resolved and subjected to immunoblotting for NPM. Total cell lysates were also loaded onto the gels to determine the extent of Degrasyn interaction with NPM. As shown in the figure, beads alone failed to recover NPM from the cell lysate while biotinylated-Degrasyn incubation recovered NPM from cell lysates. The recovery of NPM was reduced in samples co-incubated with free Degrasyn.

These results suggest that Degrasyn interacts with NPM and may be a target of Degrasyn in tumor cells.

FIG. 23 Degrasyn suppresses Stat3 activation and reduces c-myc protein levels in A375 melanoma cells.

A375 melanoma cells were pretreated with Degrasyn at the concentration noted for 2 h before the addition of 10 ng/ml IL-6 for 15 min. Cell lysates were prepared and equal protein aliquots were resolved by SDS-PAGE and immunoblotted for c-myc, p53 (wild-type), activated Stat3 (pY-Stat3), Stat3 and activated Erk1/2 kinase (p-MAPK).

Degrasyn reduced c-myc protein levels suppressed Stat3 activation at higher concentrations. Stat3, p-MAPK and p53 levels were not affected by Degrasyn. The anti-tumor sensitivity of A375 cells to Degrasyn (IC50=1.7 mM) is associated with its activity against c-myc and activated Stat3.

FIG. 24 Degrasyn suppresses the growth a A375 melanoma tumors in nude mice.

Ten Swiss nude mice (6-7 weeks of age) were injected subcutaneously (sc) with 4×10⁶ A375 melanoma cells on Day 0. When tumor nodules were palpable (Day 8), animals were divided into 2 groups. One group (Control) received 0.1 ml DMSO/PEG (1:1) intraperitoneally (ip) every other day (QD) for a total of 14 injections. The second group received 40 mg/kg Degrasyn ip beginning on Day 8 and repeated every other day for a total of 14 injections. Body weight and tumor volumes (by calipers) were measured every other day, the latter plotted vs. time. Degrasyn did not affect animal weight. All animals were euthanized when tumor volumes (in the control group) achieved the maximal allowable burden (1.5 cm³). The average +/−S.E.M. tumor volume from 5 animals per group is shown. Similar anti-tumor activity was measurable in animal receiving 80 mg/kg Degrasyn delivered by oral gavage.

Degrasyn suppresses the growth of A375 tumors in vivo.

FIG. 25 Tumor effective concentrations of Degrasyn do not suppress the growth or survival of normal human dermal fibroblasts (NDF).

Normal dermal human fibroblasts were treated for 1 h at 5 μM Degrasyn before cells were rinsed and cultured in normal growth media for 72 h. Control and treated cells were photographed after 72 h. Degrasyn did not affect the growth or survival of NDFs under these conditions. An MTT assay was performed with NDF and A375 cells under these conditions (1 h treatment, 72 h incubation) to estimate the IC50 distinctions between these populations.

Degrasyn was >5-fold more active against melanoma tumors than normal dermal fibroblast.

FIG. 26 Degrasyn stimulates tyrosine phosphorylation of specific proteins in MM-1 cells.

MM-1 cells were pretreated with Degrasyn (at the concentration indicated) for 2 h before the addition of IL-6 for 15 min (as noted). Total cell lysates were prepared and equal protein aliquots were resolved by SDS-PAGE and immunoblotted for phosphotyrosine (4G10).

Several distinct protein species (˜70 kDa, ˜55 kDa) were increased in cells treated with Degrasyn and IL-6. This activity may be a marker or mediator of Degrasyn action in IL-6 treated cells.

FIG. 27 Degrasyn stimulates tyrosine phosphorylation of specific proteins in cell lines representative of B-cell malignancies.

MM-1 and LP (lymphoma) cells were treated with Degrasyn (at the concentration indicated) for 2 h before cell lysates were prepared and equal protein aliquots were resolved by SDS-PAGE and immunoblotted for phosphotyrosine (4G10).

Several distinct protein species (˜70 kDa, ˜55 kDa) were increased in both cell lines treated with Degrasyn. This activity may be a marker or mediator of Degrasyn action in B-cell malignancies.

FIG. 28 Degrasyn stimulated tyrosine phosphorylation correlates with the anti-tumor efficacy of Degrasyn molecules.

MM-1 and Mino (Mantle cell lymphoma) cells were treated with Degrasyns with different levels of anti-tumor efficacy (at the concentration indicated) for 2 h before cell lysates were prepared and equal protein aliquots were resolved by SDS-PAGE and immunoblotted for phosphotyrosine (4G10).

Several distinct protein species (˜70 kDa, ˜55 kDa) were increased in both cell lines treated with Degrasyn. BDT-16 possesses greater anti-tumor activity than WP1130 and more potently stimulates tyrosine phosphorylation of a specific set of proteins (p70 and p55). Tyrosine phosphorylation of these proteins may be a marker or mediator of Degrasyn action in B-cell malignancies.

FIG. 29 Kinetics of Degrasyn-mediated tyrosine phosphorylation in MM-1 cells.

MM-1 cells were treated as indicated before analysis of cellular tyrosine phosphorylation by immunoblotting. Both p70 and p55 were tyrosine phosphorylated after 15 min of Degrasyn treatment which peaks in intensity after 1 h and remains higher than control levels after 4 h. These early changes may underlie or be a marker of Degrasyn activity in B-cell malignancies.

FIG. 30 Proteomic analysis of tyrosine phosphorylated p70 in Degrasyn-treated MM-1 cells.

MM-1 cells were left untreated or treated with 5 μM WP1130 for 1 h before cell lysates were prepared and directly immunoblotted for phosphotyrosine (left) or immunoprecipitated with anti-phosphotyrosine before subjecting the immune-complexed proteins to phosphotyrosine immunoblotting (middle) or silver-staining (right). This analysis was performed utilizing ˜5 mg of total protein. The protein band that migrates to the same extent as that represented by the p75 by pY immunoblotting was excised from the gel (boxed region) and subjected to in-gel trypsinization and MS analysis of the tryptic phosphopeptides as described in FIG. 20 above.

This analysis identified tyrosine phosphorylated GTPase-SH3 domain binding protein (G3BP; tyrosine phosphorylated at Y133) as the p70 protein. Bruton's tyrosine kinase (Btk) was also detected in this analysis. These proteins may play a role in Degrasyn activity in B-cell tumors.

FIG. 31 Anti-tumor activity of Degrasyn analogs.

Degrasyn analogs (structures defined herein) were incubated with MM-1 cells at the concentrations indicated for 72 h before analysis of cell growth and survival by MTT assays. The results represent the average of 4 determinations.

All compounds in this analysis were less active than WP1130 (IC50 ˜1.2 μM).

FIG. 32 HPLC spectra of certain compounds of the present invention synthesized by the solid phase resin procedure of Example 3.

FIGS. 33A-F Anti-tumor activity (IC50) of certain compounds of the present invention against MM-1 cell lines.

### DESCRIPTION OF ILLUSTRATIVE EMBODIMENTS

### I. The Present Invention

Previous studies using tyrphostin-like compounds showed inhibition of Stat3 activation, reduction in c-myc protein levels and the induction of apoptosis in tumor cells. Nielsen et al. 1997, Catlett-Falcone et al. 1999, De Vos et al. 2000, Epling-Burnette et al. 2001, Garcia et al. 2001, Liu et al. 2002, Alas and Bonavida 2003, Toyonaga et al. 2003, Barton et al., 2004, Lai et al. 2005, Shouda et al. 2006, Lee et al. 2006, Niu et al. 2002. These activities were engaged at low micromolar concentrations and occurred through an unknown mechanism. Compounds of the present invention, in certain embodiments, offer improvements over these and other previously disclosed compounds and studies. In general aspects, compounds of the present invention may induce one or more of these activities at nanomolar concentrations and typically function through a unique mechanism involving the induction of stress granules that bind specific signaling molecules and prevent them from participating in signal transduction and oncogenesis. These specific signaling molecules may comprise, in certain embodiments, Stat3, c-myc, Jak2 and/or BCR-ABL, among others.

Accordingly, the present invention provides tyrphostin-like compounds for the treatment of cell proliferative diseases such as cancer. Accordingly, in general aspects, the present invention may be described by a compound comprising the formula:

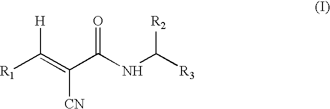

wherein:
R₁ is aryl or

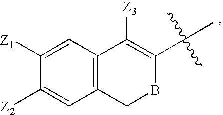

wherein
   Z₁ is H or OH;
   Z₂ is H, chloro, or —OCH₃;
   Z₃ is H or chloro; and
   B is C or 0;
R₂ is selected from the group consisting of R₄ and R₅-R₆, wherein:
   R₄ is selected from the group consisting of H, alkyl and aryl;
   R₅ is alkyl; and
   R₆ is selected from the group consisting of aryl, acyl, acyloxy, hydroxy and biotinyl; or
R₂ taken together with R₃ forms

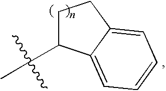

wherein n is 1-3; and
R₃ is aryl.

### II. Chemical Definitions

As used herein, the term “amino” means —NH₂; the term “nitro” means —NO₂; the term “halo” designates —F, —Cl, —Br or —I; the term “mercapto” means —SH; the term “cyano” means —CN; the term “azido” means —N₃; the term “silyl” means —SiH₃, and the term “hydroxy” means —OH.

The term “alkyl” includes straight-chain alkyl, branched-chain alkyl, cycloalkyl(alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), and

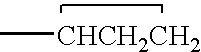

are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkanediyl” includes straight-chain alkanediyl, branched-chain alkanediyl, cycloalkanediyl, cyclic alkanediyl, heteroatom-unsubstituted alkanediyl, heteroatom-substituted alkanediyl, heteroatom-unsubstituted Cₙ-alkanediyl, and heteroatom-substituted Cₙ-alkanediyl. The term “heteroatom-unsubstituted Cₙ-alkanediyl” refers to a diradical, having a linear or branched, cyclic or acyclic structure, further having no carbon-carbon double or triple bonds, further having a total of n carbon atoms, all of which are nonaromatic, 2 or more hydrogen atoms, and no heteroatoms. For example, a heteroatom-unsubstituted C₁-C₁₀-alkanediyl has 1 to 10 carbon atoms. The groups, —CH₂— (methylene), —CH₂CH₂—, and —CH₂CH₂CH₂—, are all non-limiting examples of heteroatom-unsubstituted alkanediyl groups. The term “heteroatom-substituted Cₙ-alkanediyl” refers to a radical, having two points of attachment to one or two saturated carbon atoms, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkanediyl has 1 to 10 carbon atoms. The following groups are all non-limiting examples of heteroatom-substituted alkanediyl groups: —CH(F)—, —CH(Cl)—, —CH(OH)—, —CH(OCH₃)—, and —CH₂CH(Cl)—.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cₙ-alkenyl, and heteroatom-substituted Cₙ-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are non-limiting examples of heteroatom-substituted alkenyl groups.

The term “alkynyl” includes straight-chain alkynyl, branched-chain alkynyl, cycloalkynyl, cyclic alkynyl, heteroatom-unsubstituted alkynyl, heteroatom-substituted alkynyl, heteroatom-unsubstituted Cₙ-alkynyl, and heteroatom-substituted Cₙ-alkynyl. In certain embodiments, lower alkynyls are contemplated. The term “lower alkynyl” refers to alkynyls of 1-6 carbon atoms (that is, 1, 2, 3, 4, 5 or 6 carbon atoms). The term “heteroatom-unsubstituted Cₙ-alkynyl” refers to a radical, having a linear or branched, cyclic or acyclic structure, further having at least one carbon-carbon triple bond, a total of n carbon atoms, at least one hydrogen atom, and no heteroatoms. For example, a heteroatom-unsubstituted C₂-C₁₀-alkynyl has 2 to 10 carbon atoms. The groups, —C≡CH, —C≡CCH₃, and —C≡CC₆H₅ are non-limiting examples of heteroatom-unsubstituted alkynyl groups. The term “heteroatom-substituted Cₙ-alkynyl” refers to a radical, having a single nonaromatic carbon atom as the point of attachment and at least one carbon-carbon triple bond, further having a linear or branched, cyclic or acyclic structure, and having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkynyl has 2 to 10 carbon atoms. The group, —C≡CSi(CH₃)₃, is a non-limiting example of a heteroatom-substituted alkynyl group.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include methylphenyl, (dimethyl)phenyl, —C₆H₄CH₂CH₃, —C₆H₄CH₂CH₂CH₃, C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂,

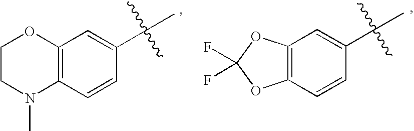

furanyl, thienyl, pyridyl, pyrrolyl, pyrimidyl, pyrazinyl, indolyl, quinolyl, and imidazoyl.

It is noted that when using terms such as phenyl, furanyl, thienyl, pyridyl, pyrrolyl, pyrimidyl, pyrazinyl, indolyl and imidazoyl herein, it is understood that these groups may be substituted or unsubstituted, unless otherwise specified. For example, the term “furanyl” encompasses, e.g., unsubstituted furanyl (C₄H₃O) and substituted furanyls (e.g., 2-methylfuranyl, 5-(4-nitrophenyl)furanyl; “phenyl” encompasses unsubstituted phenyl (—C₆H₅) and substituted phenyl (C₆H₄Br, C₆H₃NO₂Cl). An example of an “unless otherwise specified” situation occurs with, e.g., ortho-bromopyridyl: this term is restricted to this structure, with no further substitutions, as the substitution has already been fully recited (that is, the ortho-bromo substituent). As used herein, “ortho-bromopyridyl” refers to the following structure:

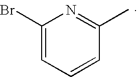

The term “aralkyl” includes heteroatom-unsubstituted aralkyl, heteroatom-substituted aralkyl, heteroatom-unsubstituted Cₙ-aralkyl, heteroatom-substituted Cₙ-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₁-aralkyl has 7 to 11 carbon atoms. Non-limiting examples of heteroatom-unsubstituted aralkyls are: 2,3-dihydro-1H-indenyl, 1,2,3,4-tetrahydronaphthalenyl, phenylmethyl (benzyl, Bn) and phenylethyl. The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms. Examples of heteroatom-substituted Cₙ-aralkyls include indolinyl, benzofuranyl and benzothiophenyl.

The term “acyl” includes straight-chain acyl, branched-chain acyl, cycloacyl, cyclic acyl, heteroatom-unsubstituted acyl, heteroatom-substituted acyl, heteroatom-unsubstituted Cₙ-acyl, heteroatom-substituted Cₙ-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cₙ-alkenyloxy, and heteroatom-substituted Cₙ-alkenyloxy. In certain embodiments, lower alkenyloxys are contemplated. The term “lower alkenyloxy” refers to alkenyloxys of 1-6 carbon atoms (that is, 1, 2, 3, 4, 5 or 6 carbon atoms). The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cₙ-alkynyloxy, and heteroatom-substituted Cₙ-alkynyloxy. In certain embodiments, lower alkynyloxys are contemplated. The term “lower alkynyloxy” refers to alkynyloxys of 1-6 carbon atoms (that is, 1, 2, 3, 4, 5 or 6 carbon atoms). The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “aryloxy” includes heteroatom-unsubstituted aryloxy, heteroatom-substituted aryloxy, heteroatom-unsubstituted Cₙ-aryloxy, heteroatom-substituted Cₙ-aryloxy, heteroaryloxy, and heterocyclic aryloxy groups. The term “heteroatom-unsubstituted Cₙ-aryloxy” refers to a group, having the structure —OAr, in which Ar is a heteroatom-unsubstituted Cₙ-aryl, as that term is defined above. A non-limiting example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “aralkyloxy” includes heteroatom-unsubstituted aralkyloxy, heteroatom-substituted aralkyloxy, heteroatom-unsubstituted Cₙ-aralkyloxy, heteroatom-substituted Cₙ-aralkyloxy, heteroaralkyloxy, and heterocyclic aralkyloxy groups. In certain embodiments, lower aralkyloxys are contemplated. The term “lower aralkyloxy” refers to alkenyloxys of 7-12 carbon atoms (that is, 7, 8, 9, 10, 11, or 12 carbon atoms). The term “heteroatom-unsubstituted Cₙ-aralkyloxy” refers to a group, having the structure —OAr, in which Ar is a heteroatom-unsubstituted Cₙ-aralkyl, as that term is defined above. The term “heteroatom-substituted Cₙ-aralkyloxy” refers to a group, having the structure —OAr, in which Ar is a heteroatom-substituted Cₙ-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cₙ-acyloxy, heteroatom-substituted Cₙ-acyloxy, alkylcarbonyloxy, arylcarbonyloxy, alkoxycarbonyloxy, aryloxycarbonyloxy, and carboxylate groups. In certain embodiments, lower acyloxys are contemplated. The term “lower acyloxy” refers to acyloxys of 1-6 carbon atoms (that is, 1, 2, 3, 4, 5 or 6 carbon atoms). The term “heteroatom-unsubstituted Cₙ-acyloxy” refers to a group, having the structure —OAc, in which Ac is a heteroatom-unsubstituted Cₙ-acyl, as that term is defined above. For example, —OC(O)CH₃ is a non-limiting example of a heteroatom-unsubstituted acyloxy group. The term “heteroatom-substituted Cₙ-acyloxy” refers to a group, having the structure —OAc, in which Ac is a heteroatom-substituted Cₙ-acyl, as that term is defined above. For example, —OC(O)OCH₃, —OC(O)NHCH₃ and —OC(O)-benzophenone are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkyl amino, heteroatom-unsubstituted Cₙ-alkylamino, and heteroatom-substituted Cₙ-alkylamino. In certain embodiments, lower alkylaminos are contemplated. The term “lower alkylamino” refers to alkylaminos of 1-6 carbon atoms (that is, 1, 2, 3, 4, 5 or 6 carbon atoms). The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cₙ-alkenylamino, heteroatom-substituted Cₙ-alkenylamino, dialkenylamino, and alkyl(alkenyl)amino groups. In certain embodiments, lower alkenylaminos are contemplated. The term “lower alkenylamino” refers to alkenylaminos of 1-6 carbon atoms (that is, 1, 2, 3, 4, 5 or 6 carbon atoms). The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cₙ-alkynylamino, heteroatom-substituted Cₙ-alkynylamino, dialkynylamino, alkyl(alkynyl)amino, and alkenyl(alkynyl)amino groups. In certain embodiments, lower alkynylaminos are contemplated. The term “lower alkynylamino” refers to alkynylaminos of 1-6 carbon atoms (that is, 1, 2, 3, 4, 5 or 6 carbon atoms). The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “arylamino” includes heteroatom-unsubstituted arylamino, heteroatom-substituted arylamino, heteroatom-unsubstituted Cₙ-arylamino, heteroatom-substituted Cₙ-arylamino, heteroarylamino, heterocyclic arylamino, and alkyl(aryl)amino groups. The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above.

The term “aralkylamino” includes heteroatom-unsubstituted aralkylamino, heteroatom-substituted aralkylamino, heteroatom-unsubstituted Cₙ-aralkylamino, heteroatom-substituted Cₙ-aralkylamino, heteroaralkylamino, heterocyclic aralkylamino groups, and diaralkylamino groups. In certain embodiments, lower aralkylaminos are contemplated. The term “lower aralkylamino” refers to aralkylaminos of 7-12 carbon atoms (that is, 7, 8, 9, 10, 11, or 12 carbon atoms). The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cₙ-amido, heteroatom-substituted Cₙ-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The group, —NHC(O)CH₃, is a non-limiting example of a heteroatom-unsubstituted amido group. The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cₙ-alkylthio, and heteroatom-substituted Cₙ-alkylthio. In certain embodiments, lower alkylthios are contemplated. The term “lower alkylthio” refers to alkylthios of 1-6 carbon atoms (that is, 1, 2, 3, 4, 5 or 6 carbon atoms). The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group. The term “heteroatom-substituted Cₙ-alkylthio” refers to a group, having the structure —SR, in which R is a heteroatom-substituted Cₙ-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cₙ-alkenylthio, and heteroatom-substituted Cₙ-alkenylthio. In certain embodiments, lower alkenylthios are contemplated. The term “lower alkenylthio” refers to alkenylthios of 1-6 carbon atoms (that is, 1, 2, 3, 4, 5 or 6 carbon atoms). The term “heteroatom-unsubstituted Cₙ-alkenylthio” refers to a group, having the structure —SR, in which R is a heteroatom-unsubstituted Cₙ-alkenyl, as that term is defined above. The term “heteroatom-substituted Cₙ-alkenylthio” refers to a group, having the structure —SR, in which R is a heteroatom-substituted Cₙ-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cₙ-alkynylthio, and heteroatom-substituted Cₙ-alkynylthio. In certain embodiments, lower alkynylthios are contemplated. The term “lower alkynylthio” refers to alkynylthios of 1-6 carbon atoms (that is, 1, 2, 3, 4, 5 or 6 carbon atoms). The term “heteroatom-unsubstituted Cₙ-alkynylthio” refers to a group, having the structure —SR, in which R is a heteroatom-unsubstituted Cₙ-alkynyl, as that term is defined above. The term “heteroatom-substituted Cₙ-alkynylthio” refers to a group, having the structure —SR, in which R is a heteroatom-substituted Cₙ-alkynyl, as that term is defined above.

The term “arylthio” includes heteroatom-unsubstituted arylthio, heteroatom-substituted arylthio, heteroatom-unsubstituted Cₙ-arylthio, heteroatom-substituted Cₙ-arylthio, heteroarylthio, and heterocyclic arylthio groups. The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group. The term “heteroatom-substituted Cₙ-arylthio” refers to a group, having the structure —SAr, in which Ar is a heteroatom-substituted Cₙ-aryl, as that term is defined above.

The term “aralkylthio” includes heteroatom-unsubstituted aralkylthio, heteroatom-substituted aralkylthio, heteroatom-unsubstituted Cₙ-aralkylthio, heteroatom-substituted Cₙ-aralkylthio, heteroaralkylthio, and heterocyclic aralkylthio groups. In certain embodiments, lower aralkylthios are contemplated. The term “lower aralkylthio” refers to aralkylthios of 7-12 carbon atoms (that is, 7, 8, 9, 10, 11, or 12 carbon atoms). The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group. The term “heteroatom-substituted Cₙ-aralkylthio” refers to a group, having the structure —SAr, in which Ar is a heteroatom-substituted Cₙ-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cₙ-acylthio, heteroatom-substituted Cₙ-acylthio, alkylcarbonyloxy, arylcarbonyloxy, alkoxycarbonyloxy, aryloxycarbonyloxy, and carboxylate groups. In certain embodiments, lower acylthios are contemplated. The term “lower acylthio” refers to acylthios of 1-6 carbon atoms (that is, 1, 2, 3, 4, 5 or 6 carbon atoms). The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group. The term “heteroatom-substituted Cₙ-acylthio” refers to a group, having the structure —SAc, in which Ac is a heteroatom-substituted Cₙ-acyl, as that term is defined above.

As used herein, the term “biotinyl” refers to a group comprising a biotin moiety. Non-limiting examples include

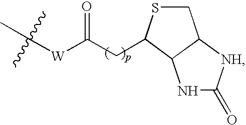

wherein W may be O or NH and p ranges from 1-10 and may, in certain embodiments, comprise an ether linkage. In certain embodiments, biotinyl is

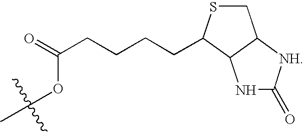

The claimed invention is also intended to encompass salts of any of the synthesized macromolecules of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred as described below, although other salts may be useful, as for example in isolation or purification steps.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphoric acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like. Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium and imidazolium.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, Selection and Use (P. H. Stahl & C. G. Wermuth eds., Verlag Helvetica Chimica Acta, 2002), which is incorporated herein by reference.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the macromolecules of the present invention are contemplated as being within the scope of the present invention. However, in certain aspects, particular diastereomers are contemplated. The chiral centers of the macromolecules of the present invention can have the S- or the R-configuration, as defined by the IUPAC 1974 Recommendations. In certain aspects, certain compounds of the present invention may comprise S- or R-configurations at particular carbon centers. For example, in the following generic formula,

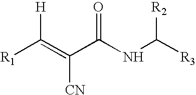

the carbon adjacent to the —NH— group and positioned between R₂ and R₃ may be preferably in the S-configuration or in the R-configuration. The present invention is meant to comprehend all such isomeric forms of the compounds of the invention.

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halo substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom to generate, for example, a heterocycloalkyl structure.

Prodrugs and solvates of the macromolecules of the present invention are also contemplated herein. The term “prodrug” as used herein, is understood as being a compound which, upon administration to a subject, such as a mammal, undergoes chemical conversion by metabolic or chemical processes to yield a compound any of the formulas herein, or a salt and/or solvate thereof (Bundgaard, 1991; Bundgaard, 1985). Solvates of the macromolecules of the present invention are preferably hydrates.

As used herein, “predominantly one enantiomer” or “substantially free” from other optical isomers means that the compound contains at least about 95% of one enantiomer, or more preferably at least about 98% of one enantiomer, or most preferably at least about 99% of one enantiomer.

The terms “AG”, “WP” “BDT”, “cp” and “MTAP”, in conjunction with a number, are descriptors used to describe certain compounds of the present invention. The terms “AG compounds,” “WP compounds” and the like similarly refer to specific examples of the present invention.

In view of the above definitions, other chemical terms used throughout this application can be easily understood by those of skill in the art. Terms may be used alone or in any combination thereof. The preferred and more preferred chain lengths of the radicals apply to all such combinations.

### III. Cell Proliferative Diseases

The term “cell proliferative diseases” refers to disorders resulting from abnormally increased and/or uncontrolled growth of cell(s) in a multicellular organism that results in harm (e.g., discomfort or decreased life expectancy) to the multicellular organism. Cell proliferative diseases can occur in animals or humans. Cancer is an example of a cell proliferative disease, and certain embodiments of the present invention are directed towards the treatment of cancer.

In certain embodiments, compounds and methods of the present invention may be used to treat a wide variety of cancerous states including, for example, melanoma, non-small cell lung, small cell lung, lung, hepatocarcinoma, retinoblastoma, astrocytoma, glioblastoma, leukemia, blood, brain, skin, eye, tongue, gum, neuroblastoma, head, neck, breast, pancreatic, renal, bone, testicular, ovarian, mesothelioma, cervical, gastrointestinal, lymphoma, colon, and/or bladder. The cancer may comprise a tumor made of cancer cells. These cancerous states may include cells that are cancerous, pre-cancerous, and/or malignant.

It is also anticipated that compounds of the present invention may also be used to treat cell proliferative diseases other than cancer. Other cell proliferative diseases that may be treated in certain embodiments of the present invention include, for example, rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (e.g., adenomatous hyperplasia, prostatic intraepithelial neoplasia), carcinoma in situ, oral hairy leukoplakia, and/or psoriasis.

Additionally, compounds of the present invention may be used to treat diseases other than hyperproliferative diseases. For example, certain tyrphostins may be useful for the treatment of hypertrophy and ischemia (U.S. Pat. No. 6,433,018) as well as hepatitis B infection (U.S. Pat. No. 6,420,338). Thus compounds of the present invention may also be useful for the treatment of other diseases including hypertrophy, ischemia, and a viral infection (e.g., hepatitis B infection).

### IV. Pharmaceutical Compositions and Administration

Compounds of this invention can be administered to kill certain cells involved in a cell proliferative disease, such as tumor cells, by any method that allows contact of the active ingredient with the agent's site of action in the tumor. They can be administered by any conventional methods available for use in conjunction with pharmaceuticals, either as individual therapeutically active ingredients or in a combination of therapeutically active ingredients. They can be administered alone but are generally administered with a pharmaceutically acceptable carrier selected on the basis of the chosen route of administration and standard pharmaceutical practice.

Aqueous compositions of the present invention will have an effective amount of the compounds to kill or slow the growth of cancer cells. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants, lipid carriers, liposomes and the like.

A. Parenteral Administration

The active compounds will often be formulated for parenteral administration, e.g., formulated for injection via the intravenous, intramuscular, subcutaneous, or even intraperitoneal routes. The preparation of an aqueous composition that contains an anthracycline of the present invention as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some forms, it will be desirable to formulate the compounds in salt form, generally to improve the solubility and bioavailability and to provide an active drug form more readily assimilated. Suitable physiologically tolerated acids are organic and inorganic acids, such as hydrochloric acid, sulfuric acid, phosphoric acid, acetic acid, citric acid, oxalic acid, malonic acid, salicylic acid, maleic acid, methane sulfonic acid, isothionic acid, lactic acid, gluconic acid, glucuronic acid, amidosulfuric acid, benzoic acid, tartaric acid and pamoaic acid. Typically, such salt forms of the active compound will be provided or mixed prior to use.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts and which are formed with inorganic acids such as, for example, hydrochloric or phosphoric acids, or such organic acids as acetic, oxalic, tartaric, mandelic, and the like.

The compounds of the present invention may also be formulated into a composition comprising liposomes or any other lipid carrier. Liposomes include: multivesicular liposomes, multilamellar liposomes, and unilamellar liposomes.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in creams and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

B. Oral Administration

In certain embodiments, active compounds may be administered orally. This is contemplated for agents which are generally resistant, or have been rendered resistant, to proteolysis by digestive enzymes. Such compounds are contemplated to include all those compounds, or drugs, that are available in tablet form from the manufacturer and derivatives and analogues thereof.

For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Upon formulation, the compounds will be administered in a manner compatible with the dosage formulation and in such amount as is therapeutically effective. The formulations are easily administered in a variety of dosage forms, such as those described below in specific examples.

### V. Therapies

One of the major challenges in oncology today is the effective treatment of a given tumor. Tumors are often resistant to traditional therapies. Thus, a great deal of effort is being directed at finding efficacious treatment of cancer. One way of achieving this is by combining new drugs with the traditional therapies. In the context of the present invention, it is contemplated that therapies using the compounds could be used in combination with surgery, chemotherapy, radiotherapy, and/or a gene therapy.

As used herein, the teem “effective” (e.g., “an effective amount”) means adequate to accomplish a desired, expected, or intended result. For example, an “effective amount” may be an amount of a compound sufficient to produce a therapeutic benefit (e.g., effective to reproducibly inhibit decrease, reduce, inhibit or otherwise abrogate the growth of a cancer cell). “Effective amounts” or a “therapeutically relevant amount” are those amounts of a compound sufficient to produce a therapeutic benefit (e.g., effective to reproducibly inhibit decrease, reduce, inhibit or otherwise abrogate the growth of a cancer cell). An effective amount, in the context of treating a subject, is sufficient to produce a therapeutic benefit. The term “therapeutic benefit” as used herein refers to anything that promotes or enhances the well-being of the subject with respect to the medical treatment of the subject's cell proliferative disease. A list of nonexhaustive examples of this includes extension of the patients life by any period of time; decrease or delay in the neoplastic development of the disease; decrease in hyperproliferation; reduction in tumor growth; delay of metastases; reduction in the proliferation rate of a cancer cell, tumor cell, or any other hyperproliferative cell; induction of apoptosis in any treated cell or in any cell affected by a treated cell; and/or a decrease in pain to the subject that can be attributed to the patient's condition.

In order to increase the effectiveness of a compound of the present invention, the compounds of the present invention may be combined with traditional drugs. It is contemplated that this type of combination therapy may be used in vitro or in vivo. For example, an anti-cancer agent, may be combined with a compound of the present invention.

This process of combining agents may involve contacting a cell(s) with the agents at the same time or within a period of time wherein separate administration of the substances produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a compound of the present invention is “A” and a second agent, such as an anti-cancer agent, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A. | | | | |

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All chemicals and solvents were obtained from Sigma-Aldrich (Milwaukee, Wis.) or Fisher Scientific (Pittsburgh, Pa.) and used without further purification. The compounds, agents, and active ingredients (e.g., solvents, catalysts, bases used in reactions, and other compounds, agents, and active ingredients described herein) that are described in the claims and specification can be obtained by any means known to a person of ordinary skill in the art.

¹H-NMR and ¹³C-NMR spectra were recorded on an IBM-Brucker Avance 300 (300 MHz for ¹H-NMR and 75.48 MHz for ¹³C-NMR), and IBM-Brucker Avance 500 (500 MHz for ¹H-NMR and 125.76 MHz for ¹³C-NMR) or Brucker Biospin spectrometer with a B-ACS 60 autosampler (600.13 MHz for ¹H-NMR and 150.92 MHz for ¹³C-NMR) spectrometers. Chemical shifts (δ) are determined relative to CDCl₃ (referenced to 7.27 ppm (δ) for ¹H-NMR and 77.0 ppm for ¹³C-NMR) or DMSO-d₆ (referenced to 2.49 ppm (δ) for ¹H-NMR and 39.5 ppm for ¹³C-NMR). Proton-proton coupling constants (J) are given in Hertz and spectral splitting patterns are designated as singlet (s), doublet (d), triplet (t), quadruplet (q), multiplet or overlapped (m), and broad (br). Low resolution mass spectra (ionspray, a variation of electrospray) were acquired on a Perkin-Elmer Sciex API 100 spectrometer or Applied Biosystems Q-trap 2000 LC-MS-MS. Flash chromatography was performed using Merck silica gel 60 (mesh size 230-400 ASTM) or using an Isco (Lincoln, Nebr.) combiFlash Companion or SQ16x flash chromatography system with RediSep columns (normal phase silica gel, mesh size 230-400 ASTM) and Fisher Optima™ Dude solvents. Thin-layer chromatography (TLC) was performed on Merck (Darmstadt, Germany) silica gel F-254 aluminum-backed plates with visualization under UV (254 nm) and by staining with potassium permanganate or ceric ammonium molybdate. Analytical HPLC was performed on a Varian Prostar system, with a Varian Microsorb-MW C18 column (250×4.6 mm; 5μ) using the following solvent system A=H₂O/0.1% TFA and B=acetonitrile/0.1% TFA. Varian Prepstar preparative system equipped with a Prep Microsorb-MW C18 column (250×41.4 mm; 6μ; 60 Å) was used for preparative HPLC with the same solvent systems. Program A: Gradient: 0-5 min. 30% B. 5-35 min. 95% B. 30-35 min. 95% B. Program B: Gradient: 0-5 min. 10% B. 5-35 min. 95% B. 30-40 min. 95% B. UV was measured on Perkin Elmer Lambda 25 UV/Vis spectrometer. Solid phase synthesis was performed on an apptec apex396 combinatory synthesizer. IR was measured on Perkin Elmer Spectra One FT-IR spectrometer.

### Example 1

### Synthesis and Molecular Modeling of Degrasyn Libraries

In order to better explore the chemical space surrounding the current structural template, the diversity of chemical building blocks was explored. In one synthetic procedure, aromatic amines and aromatic aldehydes were utilized as the building blocks. A search was conducted in silico using a database of available compounds from 49 different chemical database vendors. All modeling work was completed on a 4-processor SGI Tezro using the Sybyl Modeling Suite from Tripos, Inc. These compounds had previously been converted to a 2D/3D searchable database with the Unity package in Sybyl and a C-shell script was used to search each one in series using the 2D search feature of the dbsearch command. The 2D structures and Sybyl Line Notation (SLN) for the queries are as shown:

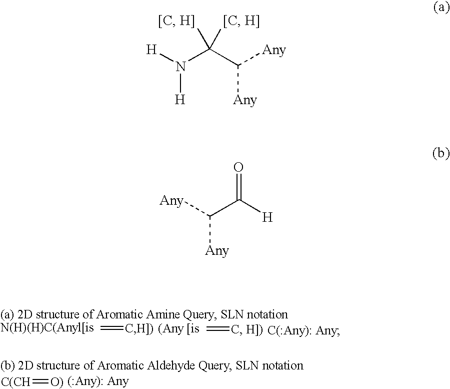

The search for aromatic amines resulted in 5,541 compounds, which was further reduced to 3,084 by screening out compounds with MW>250, mixtures, those that contained metals or isotopes, or lacked 3D coordinates. This was completed with the dbslnfilter command, which is part of the O/S utilities. The list of compounds was read into a molecular spreadsheet and the Selector module was used to reduce this to 100 compounds. This involved using the Jarvis-Patrick method to obtain the diversity clusters using the calculated Atom pairs and 2D Fingerprint metrics and selecting 100 compounds randomly from these clusters. This diverse set of compounds was then searched for physical availability. Utilizing the same approach, the search for aromatic aldehydes resulted in 29,245 compounds, which was reduced to 7,946 by MW only. The same diversity technique was used to reduce this to only 100 compounds, which were then search for physical availability. Compounds were ordered from these lists and they foamed a portion of the building blocks for the synthesis of compounds described in the sections on synthetic procedures.

### Example 2

### General Procedure for Synthesizing Certain Compounds of the Present Invention

Scheme 1, shown below, represents a general synthetic procedure for the synthesis of certain compounds of the present invention, wherein R₁-R₆ may comprise one or more of any substituent as described herein, and X may be N or C (WO 1995/028922). For example, equimolar amounts of benzylamine and cyanoacetic methyl ester quantitively react to form N-benzylcyanoacetamide as an intermediate, then Knoevenagel condensation with benzaldehyde furnishes the final product. Over sixty compounds of the present invention have been prepared via this route.

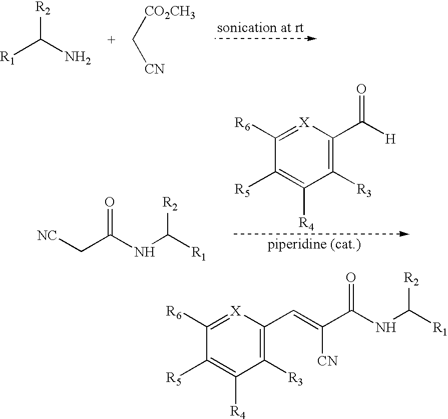

### Example 3

### Example of A Synthetic Preparation of Certain Compounds of the Present Invention

### Solid Phase—Resin

The following scheme represents an exemplary method of preparing certain compounds of the present invention. This procedure is based on a literature preparation (Gu et al., 2005).

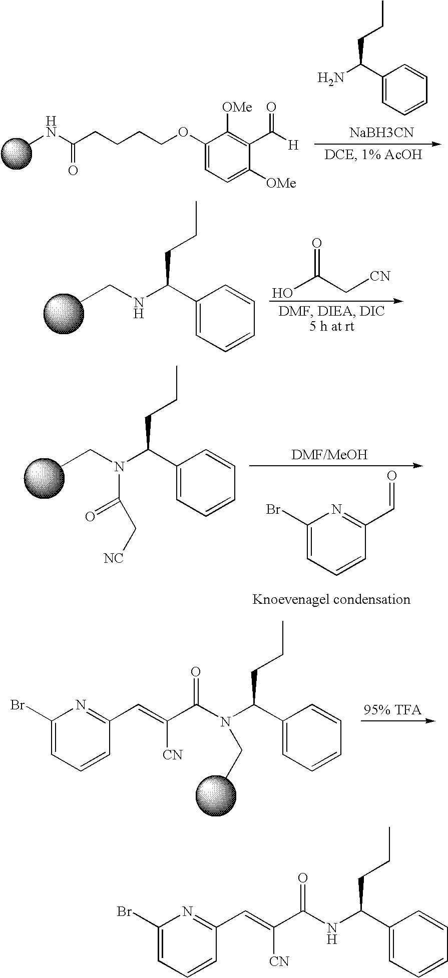

General Procedure for Reductive Amination Using Asymmetric Aromatic Amines with BAL-PG-PS Resin

BAL-PG-PS Resin (1 g), NaBH₃CN (1.56 g, 25 equiv), DCE (35 mL), 2-phenylethylamine (3.25 mL, 25 equiv) and AcOH (0.38 mL, 4 equiv) was rotated on orbital shaker for 24 h. The resultant secondary amine resin was washed with DCM (10 mL×10) and DMF-CH₂Cl₂ (1:1) (10 mL×10) and dried well. A ninhydrin test confirmed the completion of the reaction.

### General Procedure for Cyanoacylation of Secondary Amine:

A solution of cyanoacetic acid (3 g, 20 equiv), DIPCDI (20 equiv) and DIPEA (20 equiv) in DMF (30 mL) was added to the above resin and rotated on an orbital shaker for 7 h. The resin was washed with DMF (20 mL×10) and the acylation was repeated for another 7 h. The resin was washed and dried. A ninhydrin test confirmed the completion of the reaction.

### General Procedure for Knoevenagel Condensation:

A solution of aldehyde (5 equiv) in anhydrous DMF/EtOH (10:2) (1.5 mL) and 10 drops of piperidine was added to the above resin in 96 wells and shaken over night. The reaction mixture was drained, and the resin was washed with DMF (1.5 mL×10).

### General Procedure for Final Cleavage:

All products were cleaved from the resin with 95% TFA in water for 1.5 h and collected from 96-well deep well blocks. The solvent was removed in vacuo, and the residue was dissolved in CH₃CN and subjected to purification by reverse phase HPLC using H₂O (0.1% TFA) and CH₃CN (0.1%) as eluent (10-90% gradient) and analyzed by LC/MS.

### Example 4

### Exemplary Compounds Synthesized by the Solid Phase Resin Procedure of Example 3

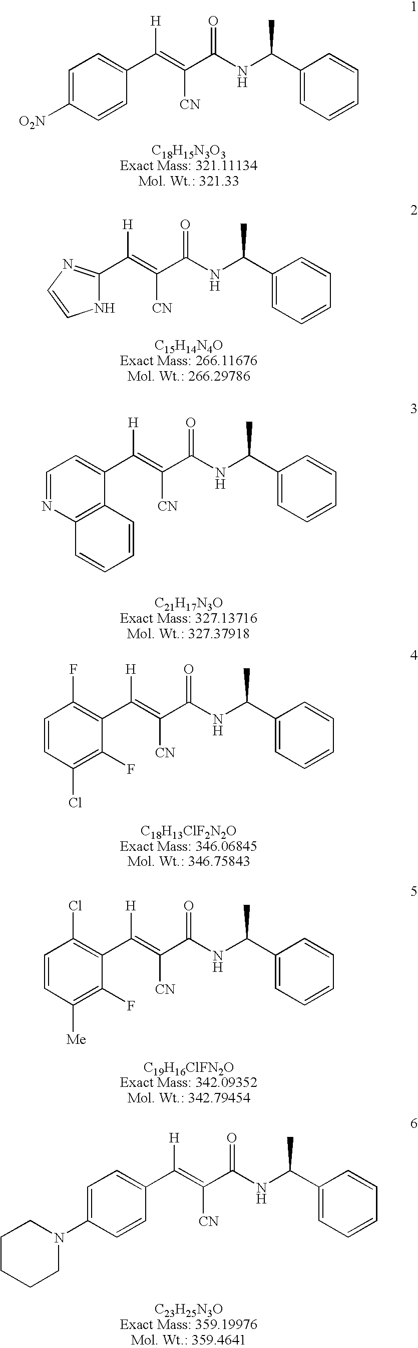

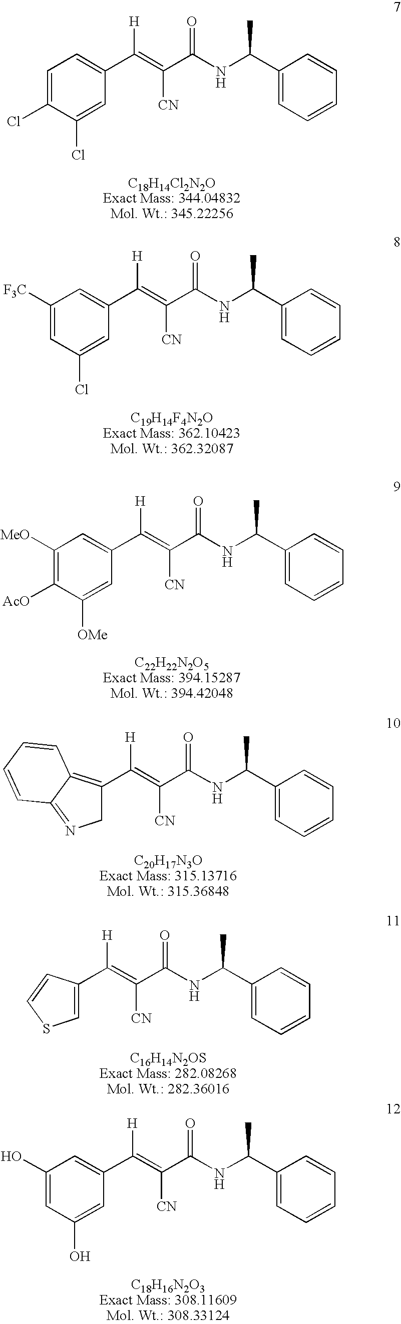

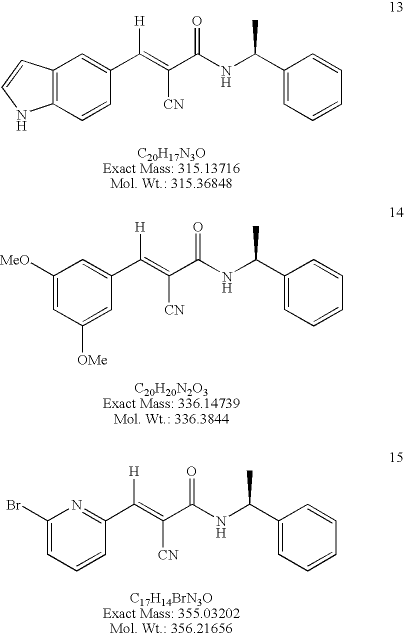

Compounds 2, 4, 5, 6, 7 and 20 were confirmed by mass spectrometry.

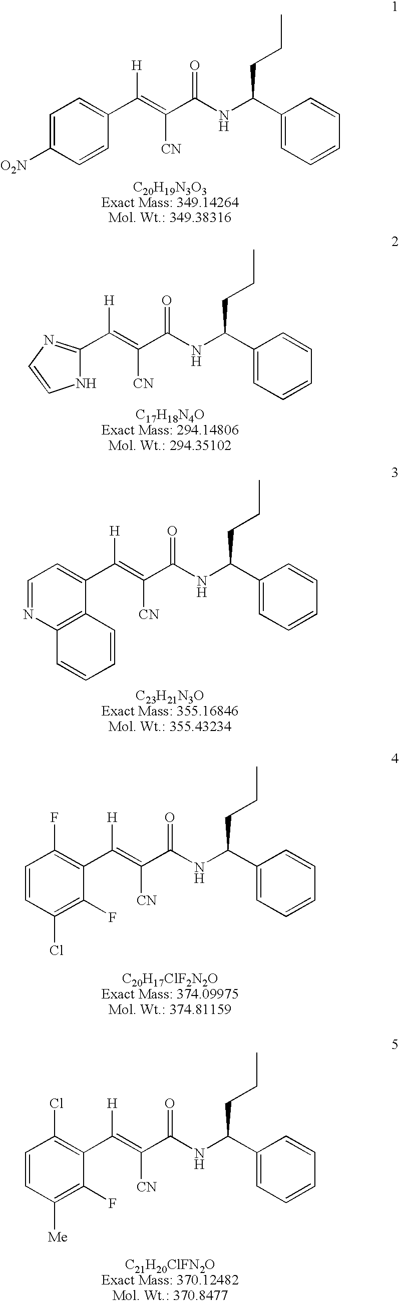

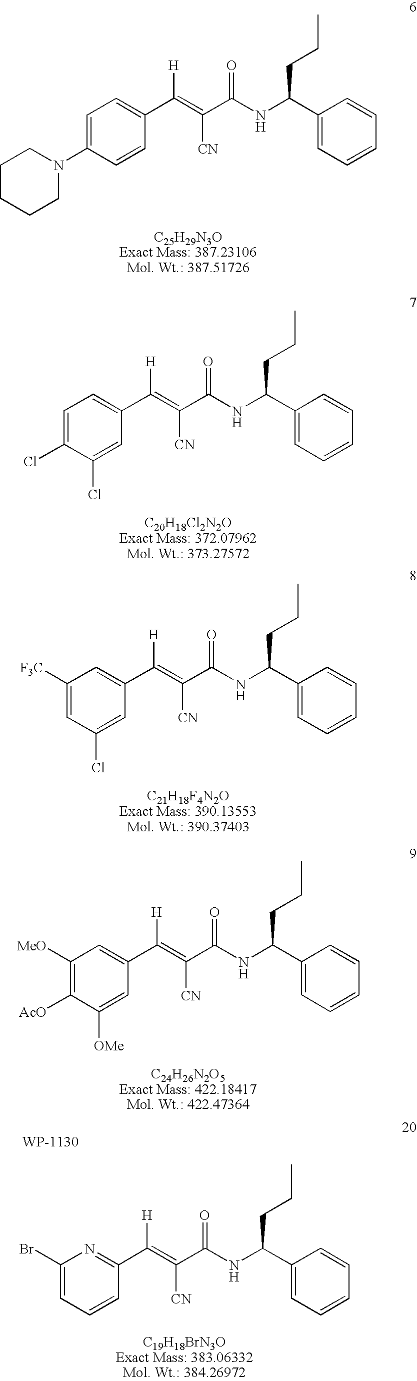

Compounds 6, 9 and 20 were confirmed by mass spectrometry.

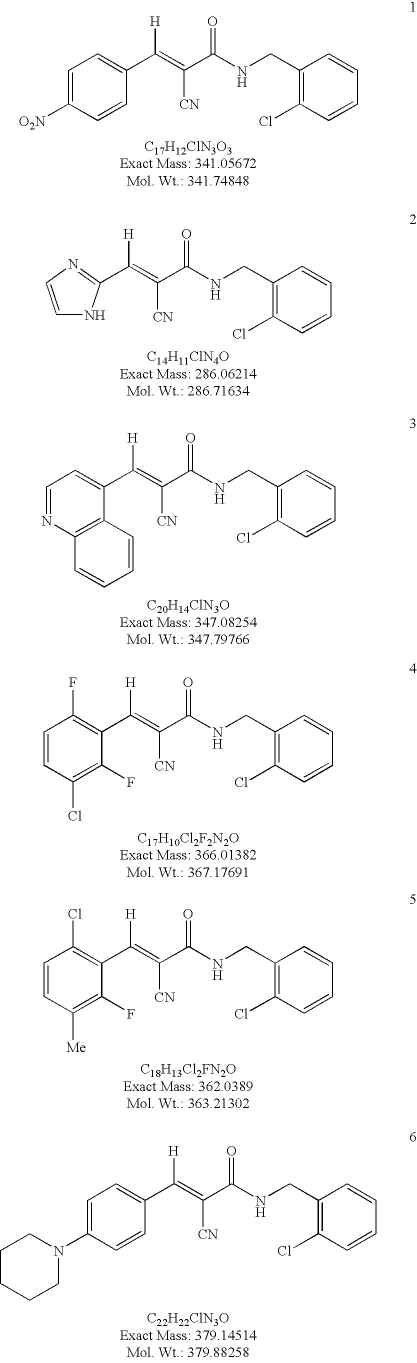

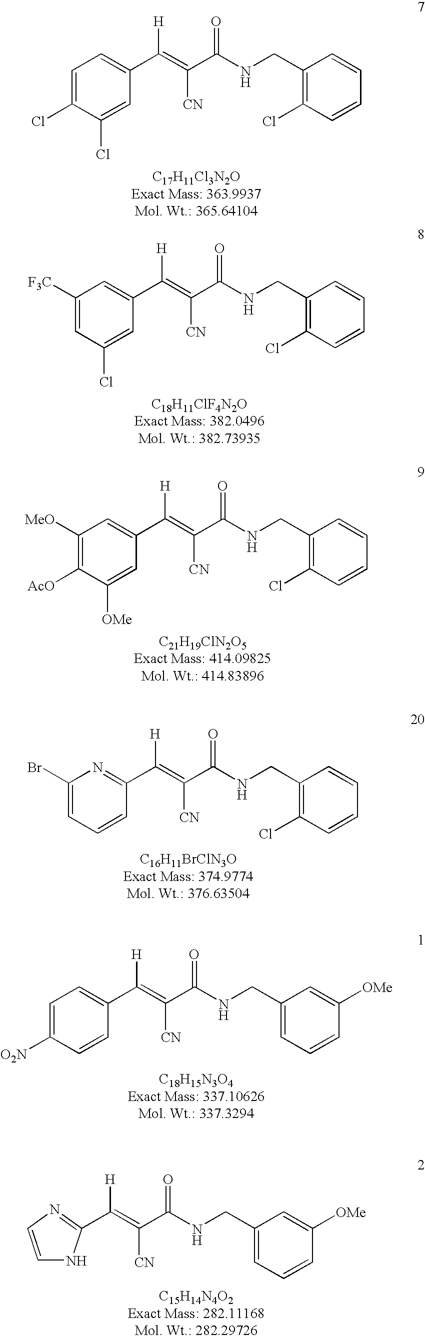

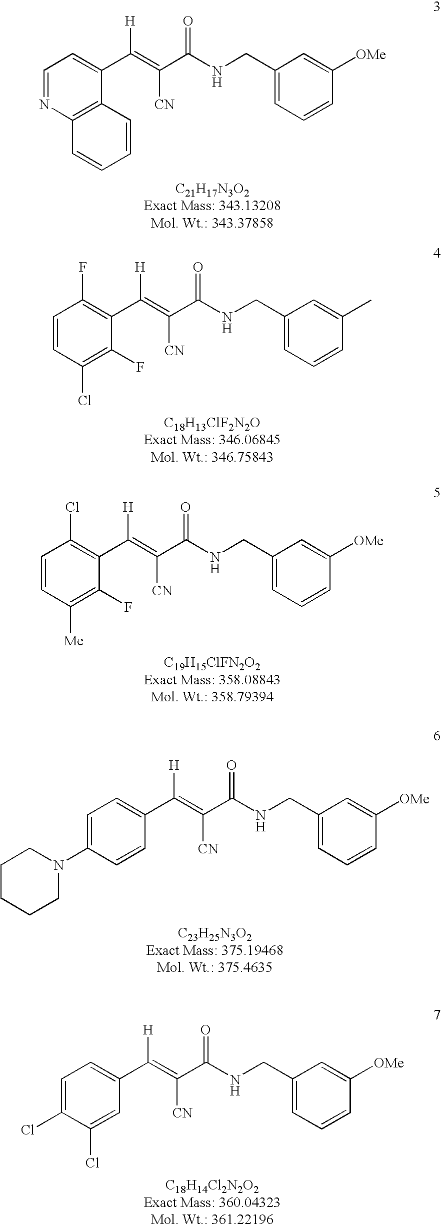

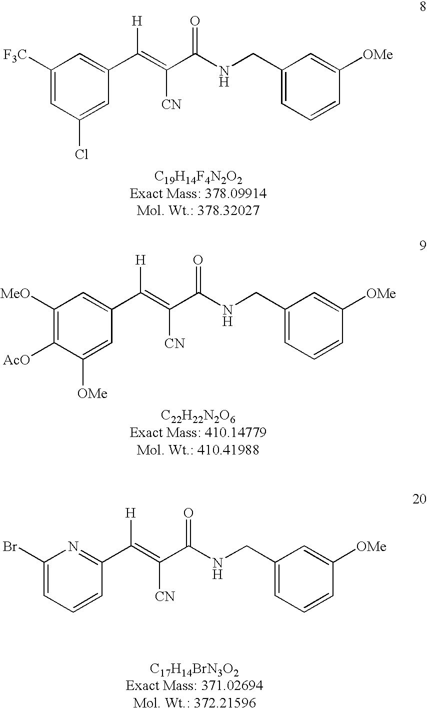

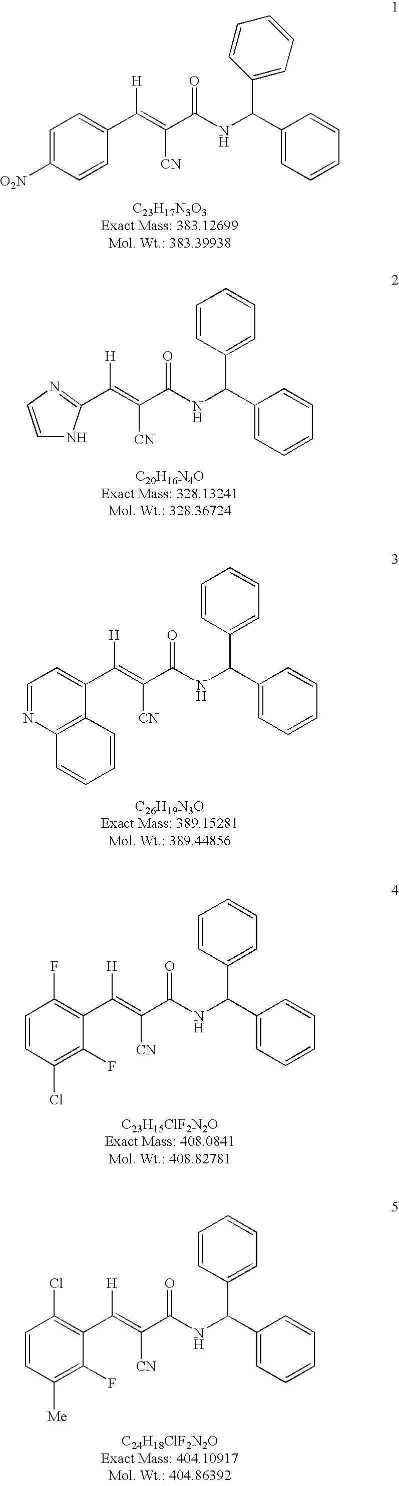

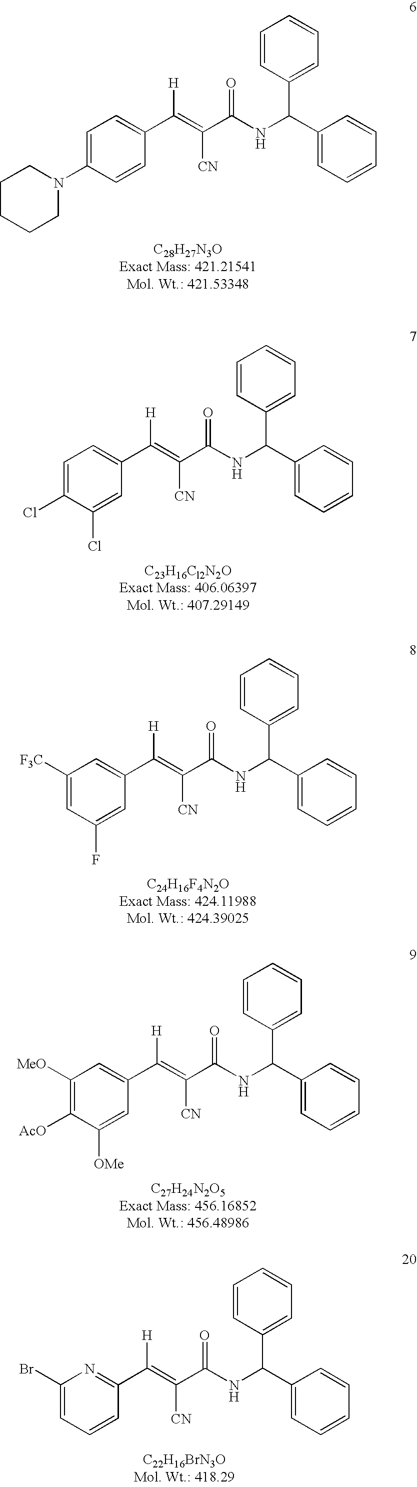

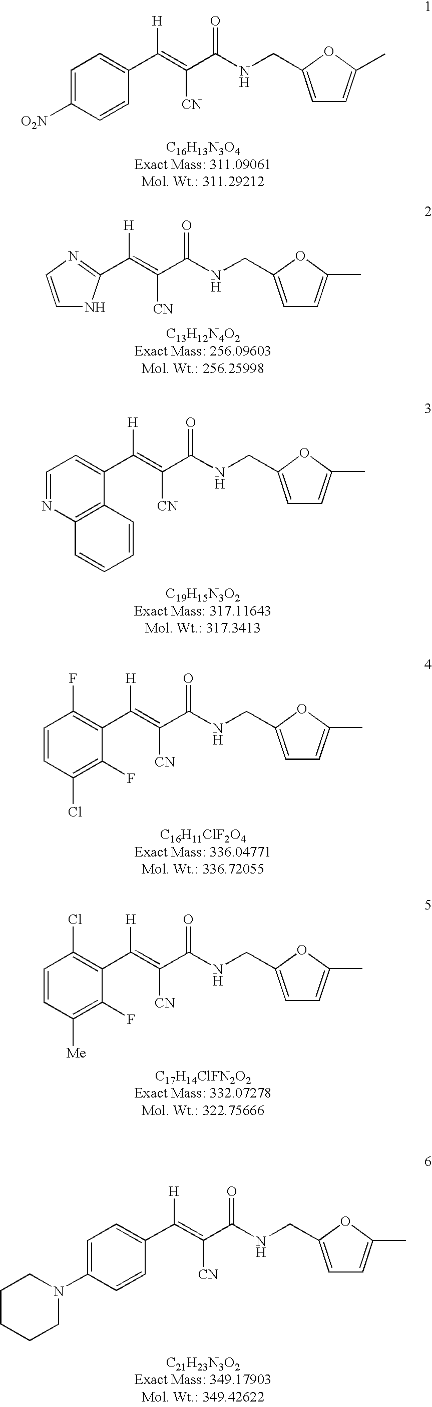

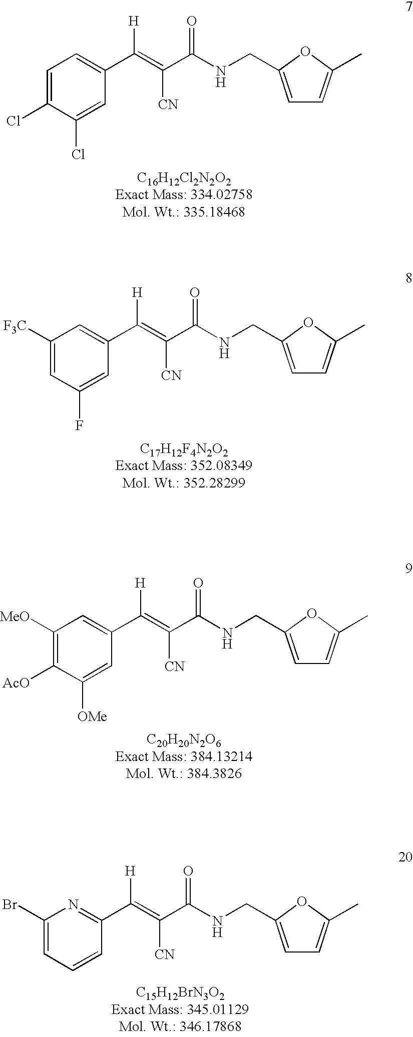

### Example 5

### Example of a Synthetic Preparation of Certain Compounds of the Present Invention

### Solid Phase—Lanterns

The following scheme represents an exemplary method of preparing certain compounds of the present invention. This procedure is based on a literature preparation (Gu et al., 2005).

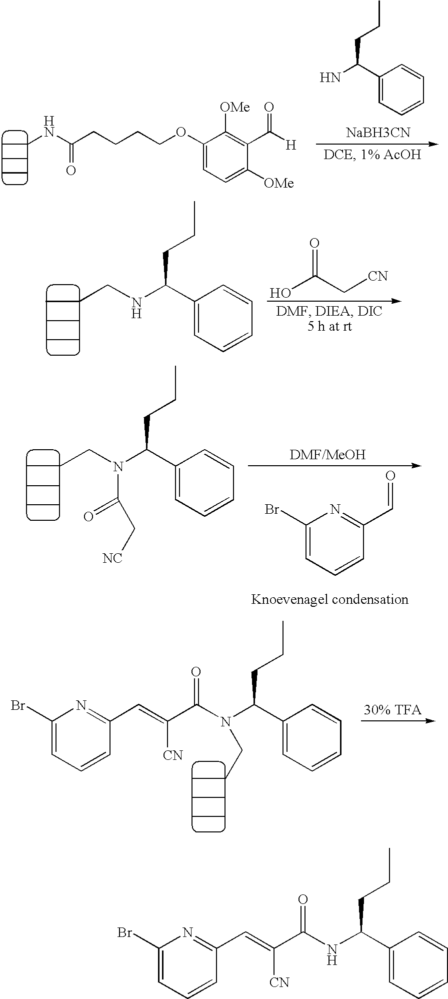

### General Procedure for Reductive Amination:

BAL Lanterns (A-series) (initial specified loading: 750 μmol) are treated with 11 mL of a solution of amine (0.5 M, 5.3 mmol, 7 mole equivalents) and sodium cyanoborohydride (0.05 M, 530 μmol, 0.7 mole equivalents) in 1% acetic acid/DMF at 60° C. for 17 h. After cooling to rt, the reagent solution is decanted and the Lanterns washed with DMF (3×3 min) and DCM (3×3 min) (20 ml).

### General Procedure for Cyanoacylation of Secondary Amine.

### See Example 3.

### General Procedure for Knoevenagel Condensation:

### See Example 3.

### General Procedure for Final Cleavage:

All products were cleaved from the Lantern with 30% TFA in water for 1.5 h and collected from 96-well deep well blocks. The solvent was removed in vacuo, and the residues were dissolved in CH₃CN and subjected to purification by reverse phase HPLC using H₂O (0.1% TFA) and CH₃CN (0.1%) as eluent (10-90% gradient) and analyzed by LC/MS.

### Example 6

### Exemplary Compounds Synthesized by the Solid Phase Lantern Procedure of Example 5

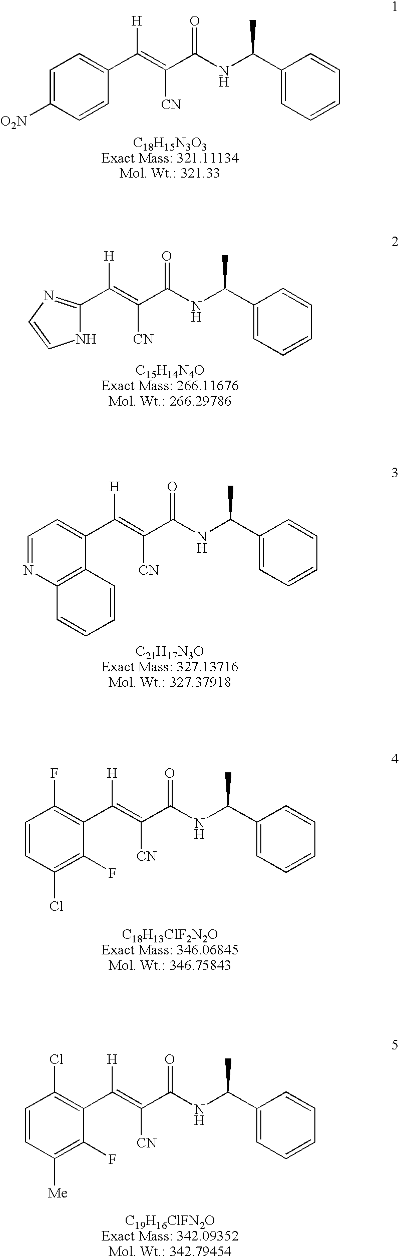

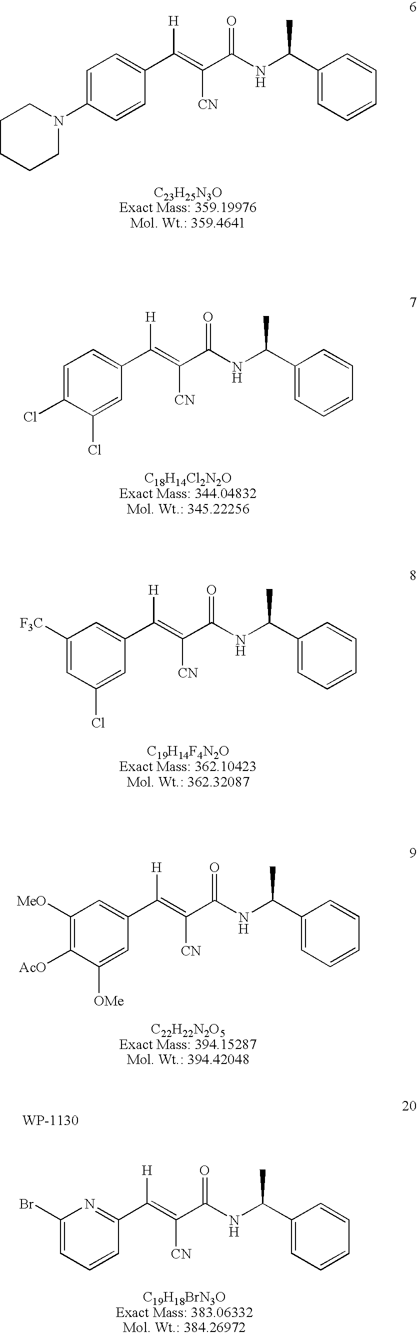

The structures of compound 20, compound 6 and compound 9 were confirmed using mass spectrometry.

### Example 7

### Exemplary Aldehydes and Amines that May Employed to Generate Compounds of the Present Invention

### Such as in the Procedures of Examples 3 and 5

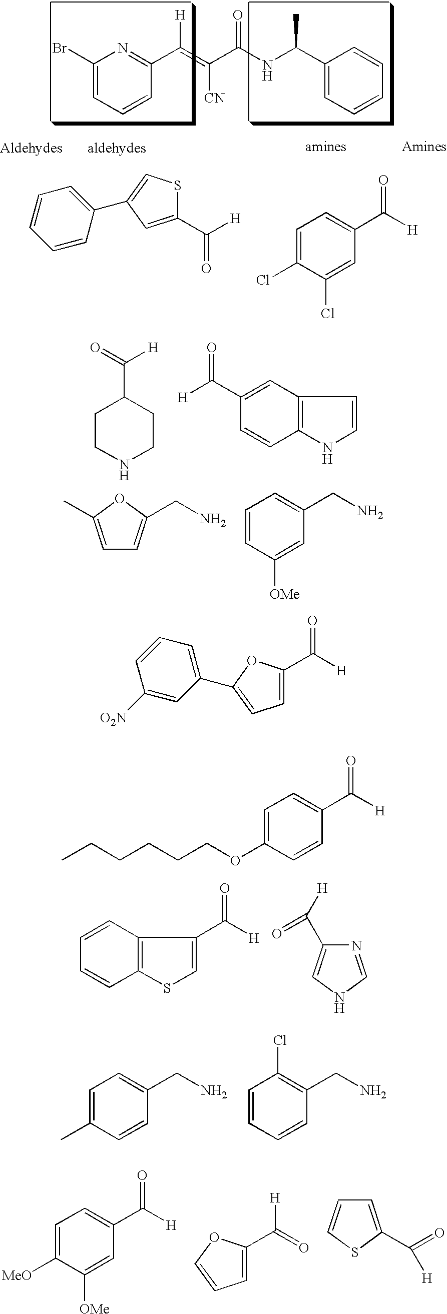

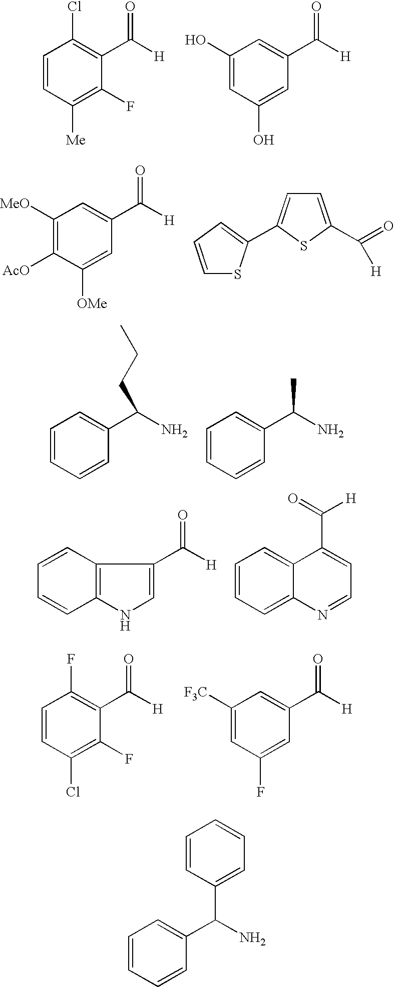

### Example 8

### Representative Synthesis and NMR Characterization of a Biotin-Containing Compound of the Present Invention

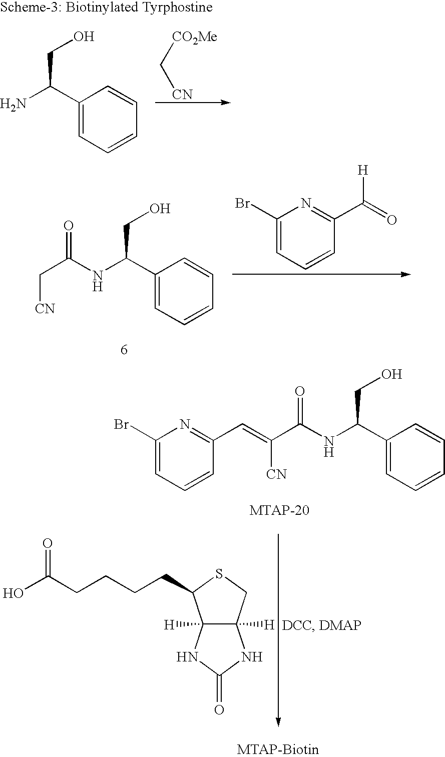

N-(Cyanoacetyl)-2-hydroxyl-1-phenylethyl amide (6). A mixture of methyl cyanoacetate (2.47 g, 25 mmol) and 2-amino-2-phenylethylamine (3.43 g, 25 mmol) was stirred vigorously for overnight. The resulting solid was triturated with 8 mL 95% ethanol and the product filtered as a white solid (4.28 g, 85% yield). ¹H NMR (600 MHz, DMSO-d₆) δ 8.69 (d, 2H, J=8.4 Hz), 7.30 (m, 5H), 4.97 (bs, 1H), 4.81 (q, 1H, J=4.8 Hz), 3.71 (m, 2H), 3.55 (m, 2H).

2-Propenamide, 2-cyano-3-(3-bromo 2-pyridinyl)-N-(1-phenyl-1-ethylhydroxyl)-(E) (MTAP-20). The amide 6 from the previous reaction (1.06 g, 5.2 mmol), 6-bromo-pyrrodocarboxyldehyde (0.97 g, 5.2 mmol) and piperidine (five drops) were stirred in anhydrous ethanol (10 mL). Ethanol was evaporated and solid was triturated with water and dried under high vacuum to give (1.70 g, 88% yield) of the desired product 6 as a white solid. ¹H NMR (600 MHz, CDCl₃) δ 8.21 (s, 1H), 7.68 (t, 1H, J=7.8 Hz), 7.60 (m, 2H), 7.35 (m, 5H), 7.01 (d, 2H, J=8.4 Hz), 5.22 (quin, 1H, J=7.2 Hz), 4.00 (m, 2H), 2.13 (bs, 1H). ¹³C NMR δ 159.6, 150.8, 148.6, 142.45, 139.2, 137.9, 130.8, 129.1, 127.7, 128.2, 126.7, 126.7, 125.5, 115.7, 109.3, 65.9, 56.5. MS (C₁₇H₁₄BrN₃O₂) estimated 371.02; found 372.3 and 374.2.

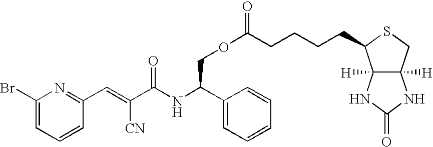

MTAP-Biotin: 2-Propenamide, 2-cyano-3-(3-bromo 2-pyridinyl)-N-(2-phenyl-2-ethylene)-1-biotinyl ester-(E). 2-Propenamide, 2-cyano-3-(3-bromo-2-pyridinyl)-N-(1-phenyl-1-ethylhydroxyl) (90 mg, 0.25 mmol), D-biotin (vitamin H) (118 mg, 0.5 mmol), DCC (76 mg, 0.37 mmol) and DMAP (40 mg, 0.32 mmol) were stirred in dry CH₂Cl₂ (20 mL) for 24 h. The resulting solution was dried and purified by flash chromatography with 30 to 80% ethyl acetate in hexane as eluent. A white solid resulted (yield: 95 mg, 75%). ¹H NMR (600 MHz, DMSO-d₆) δ 9.18 (d, 1H, J=8.4 Hz), 8.12 (s, 1H), 7.96 (t, 1H, J=7.8 Hz), 7.91 (d, 1H, J=7.2 Hz), 7.82 (d, 1H, J=7.8 Hz), 7.46 (d, 2H, J=7.8 Hz), 7.39 (t, 2H, J=7.2 Hz), 7.32 (t, 1H, J=7.2 Hz), 6.43 (s, 1H), 6.36 (s, 1H), 5.27 (m, 1H), 4.33 (m, 3H), 4.10 (m, 1H), 3.05 (m, 1H), 2.79-2.5 (m, 2H), 2.32 (m, 2H), 1.58-1.26 (m, 6H); ¹³C NMR δ 173.6, 163.9, 159.7, 150.8, 148.7, 142.3, 139.4, 137.4, 130.8, 128.9, 128.9, 128.3, 126.7, 126.7, 125.9, 115.6, 109.3, 65.8, 61.8, 60.2, 58.2, 55.5, 53.7, 40.5, 33.8, 28.2, 24.8. MS (C₂₇H₂₈BrN₅O₄S) estimated 598.51; found 598.3 and 600.3.

### Example 10

### A Representative Synthesis and NMR Characterization of an Azide-Containing Compound of the Present Invention

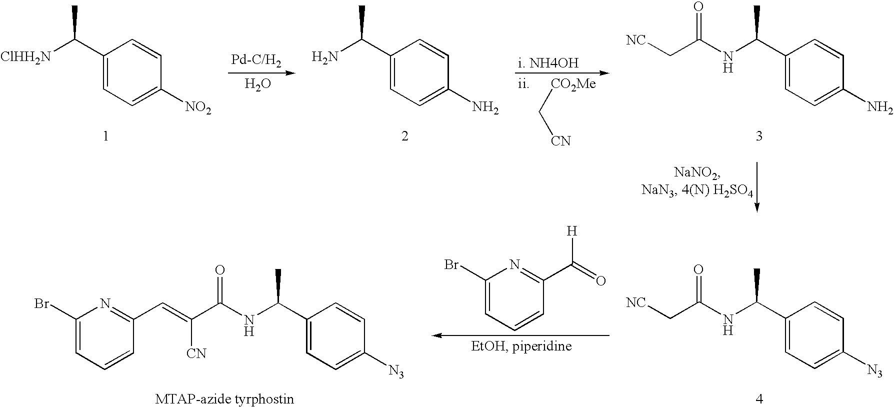

1-(4′-aminophenyl)ethylamine (2). 1-(4′-nitrophenyl)-ethylamine hydrochloride (2.06 g, 10 mmol) was dissolved in distilled water (20 mL) and 200 mg of Pd—C were added. The mixture was hydrogenated (40 psi) for 6 h. After filtration through celite, saturated ammonium hydroxide in brine (50 mL) was added and extracted with EtOAc. The solvent was removed and dried yield 90% (1.23 g) as a light yellow liquid. ¹H NMR (600 MHz) δ 7.12 (dt, 2H, J=2.6, 8.2 Hz), 6.65 (dt, 2H, J=2.6, 8.2 Hz), 4.01 (q, 1H, J=6.6 Hz), 1.35 (d, 3H, 3.2 Hz).

N-(Cyanoacetyl)-1-(4′-aminophenyl)ethyl amide (3). A mixture of methyl cyanoacetate (2.47 g, 25 mmol) and 1-(4′-aminophenyl)ethylamine (3.40 g, 25 mmol) was stirred vigorously overnight. The resulting solid was triturated with 8 mL 95% ethanol and the product filtered as a white solid (4.08 g, 80% yield). ¹H NMR (600 MHz, CD₃OD) δ 7.12 (dt, 2H, J=2.9, 8.3 Hz), 6.71 (dt, 2H, J=2.9, 8.3 Hz), 4.94 (q, 1H, J=6.7 Hz), 3.2 (s, 2H), 1.43 (d, 3H, 6.4 Hz); ¹³C NMR δ 162.4, 146.9, 132.7, 127.0, 127.0, 115.6, 115.6, 49.4, 20.9.

N-(Cyanoacetyl)-1-(4′-azidophenyl)ethyl amide (4). Cyanoacetyl amide 3 (2.56 g 12.6 mmol) was taken up in 4 N H₂SO₄ (15 mL) at 0° C., affording a reddish suspension. NaNO₂ (1.3 g, 18.9 mmol) was added as a solution in water (10 mL) open to the air. The reaction mixture was maintained at 0° C. for 15 min with the solution clearing. NaN₃ (1.23 g, 18.9 mmol) in water (10 mL) was added slowly with gas evolution. The reaction mixture was stirred at room temperature for 1 h, resulting in a brownish-white precipitation. The mixture was extracted with CH₂Cl₂ and the organic layers were dried with MgSO₄ and concentrated under reduced pressure. Flash chromatography with EtOAc/hexanes (40 to 60%) afforded 2.6 g (90%) of a white solid. ¹H NMR (600 MHz, CDCl₃) δ 7.31 (dt, 2H, J=3.0, 8.4 Hz), 7.02 (dt, 2H, J=3.0, 8.4 Hz), 6.25 (bs, 1H), 5.09 (quin, 1H, J=6.0 Hz), 3.36 (s, 2H), 1.57 (d, 3H, 7.0 Hz).

2-Propenamide, 2-cyano-3-(3-bromo 2-pyridinyl)-N-[1-[4-azidophenyl]ethyl]-(E) (MTAP-azide tyrphostin) The amide 4 from the previous reaction (1.2 g, 5.2 mmol), 6-bromo-pyridinecarboxyldehyde (0.97 g, 5.2 mmol) and piperidine (five drops) were stirred in anhydrous ethanol (10 mL). Ethanol was evaporated and solid was triturated with water and dried under high vacuum to give (1.7 g, 82% yield) of the desired product as a white solid. ¹H NMR (600 MHz, CDCl₃) δ 8.18 (s, 1H), (t, 1H, J=7.8 Hz), 7.58 (d, 1H, 4.2 Hz), 7.56 (d, 1H, J=3.6 Hz), 7.35 (d, 2H, J=8.4 Hz), 7.01 (d, 2H, J=8.4 Hz), 5.20 (p, 1H, J=7.2 Hz), 1.59 (d, 3H, J=4.3 Hz); ¹³C NMR δ 158.7, 150.8, 148.4, 142.45, 139.5, 139.16, 130.7, 127.7, 127.7, 125.5, 119.4, 119.4, 119.3, 115.7, 109.3, 49.8, 21.6. MS (C₁₇H₁₃BrN₆O) estimated 396.033; found 395.1 and 397.1 (M−H) for bromine isotopes.

### Example 11

### Spectral Characterization of a Compound of the Present Invention

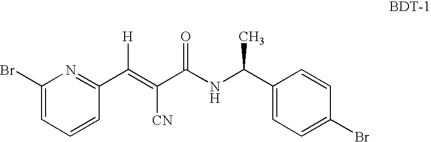

¹H NMR (500 MHz, CDCl₃) δ 1.59 (d, 3H, J=11.8 Hz), 5.18 (m 1H), 6.78 (d, 1H, J=6.5 Hz), 7.24 (d, 2H, J=8.4 Hz), 7.48 (d, 2H, J=8.4 Hz), 7.57 (m, 2H), 7.66 (m, 1H), 8.18 (s, 1H); ¹³C NMR (125 MHz, CDCl₃) δ 21.63, 49.78, 125.56, 127.91, 130.76, 131.95, 139.16, 148.47, 150.83, 158.61; MS (ESI) m/e (rel intensity): 433.8 (50), 435.9 (100), 437.8 (50).

### Example 12

### Spectral Characterization of a Compound of the Present Invention

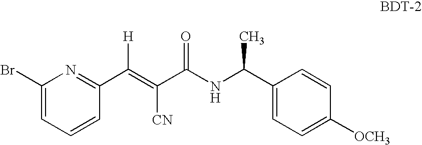

¹H NMR (300 MHz, CDCl₃) δ 1.60 (d, 3H, J=6.8 Hz), 3.82 (s, 1H), 5.22 (m 1H), 6.73 (d, 1H, J=6.8 Hz), 6.91 (d, 2H, J=8.6 Hz), 7.29 (m, 2H), 7.63 (m, 3H), 8.22 (s, 1H); ¹³C NMR (75 MHz, CDCl₃) δ 21.94, 50.09, 55.72, 109.95, 114.63, 125.78, 127.80, 131.02, 134.50, 139.52, 148.64, 151.33, 158.79, 159.56; MS (ESI) m/e (rel intensity): 386.3 (16), 388.3 (16).

### Example 13

### Spectral Characterization of a Compound of the Present Invention

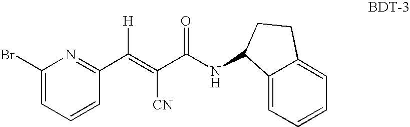

¹H NMR (500 MHz, CDCl₃) 6, 1.95 (m, 1H), 2.70 (m, 1H), 2.94 (m, 1H), 3.07 (m 1H), 5.62 (m, 1H), 6.74 (d, 1H, J=7.8 Hz), 7.28 (m, 4H), 7.58-7.69 (m, 3H), 8.28 (s, 1H); ¹³C NMR (125 MHz, CDCl₃) δ 30.31, 33.82, 55.97, 109.50, 115.67, 124.06, 125.03, 125.42, 127.08, 128.48, 130.68, 139.16, 141.88, 142.51, 143.44, 148.42, 150.96, 159.22; MS (ESI) m/e (rel intensity): 368.2 (30), 370.2 (30).

### Example 14

### Spectral Characterization of a Compound of the Present Invention

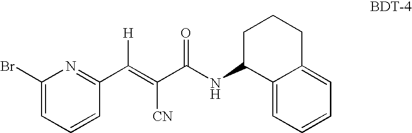

¹H NMR (500 MHz, CDCl₃) 6, 1.90 (m, 3H), 2.15 (m, 1H), 2.79 (m, 1H), 2.88 (m 1H), 5.31 (m, 1H), 6.74 (d, 1H, J=8.1 Hz), 7.13 (d, 1H, J=7.4 Hz), 7.19 (m, 2H), 7.58 (d, 1H, J=8.5 Hz), 7.59-7.69 (m, 2H), 8.29 (s, 1H); ¹³C NMR (125 MHz, CDCl₃) δ 20.02, 29.13, 29.99, 48.99, 109.69, 115.64, 125.35, 126.54, 127.78, 128.47, 129.43, 130.64, 135.31, 137.65, 139.15, 142.49, 148.40, 151.00, 158.76; MS (ESI) m/e (rel intensity): 382 (50), 384 (50).

### Example 15

### Spectral Characterization of a Compound of the Present Invention

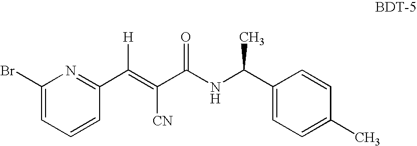

¹H NMR (500 MHz, CDCl₃) δ 1.60 (d, 3H, J=6.9 Hz), 2.34 (s, 3H), 5.20 (m 1H), 6.75 (d, 1H, J=7.3 Hz), 7.17 (d, 2H, J=8.0 Hz), 7.25 (m, 2H), 7.58 (m, 2H), 7.65 (t, 1H, J=7.5 Hz), 8.20 (s, 1H); ¹³C NMR (125 MHz, CDCl₃) δ 21.07, 21.67, 50.03, 109.56, 115.73, 125.40, 126.07, 129.55, 130.62, 137.53, 139.07, 139.13, 142.47, 148.36, 150.95, 158.44; MS (ESI) m/e (rel intensity): 370.2 (47), 372.2 (50).

### Example 16

### Spectral Characterization of a Compound of the Present Invention

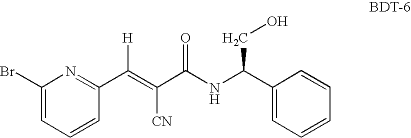

¹H NMR (500 MHz, DMSO-d₆) δ 3.68 (m, 2H), 4.97 (m 1H), 5.02 (t, 1H, J=5.7 Hz), 7.26 (t, 1H, J=7.2 Hz), 7.34 (m, 2H) 7.38 (d, 2H, J=7.2 Hz), 7.80 (d, 1H, J=7.8 Hz), 7.89 (d, 1H, J=7.3 Hz), 7.95 (t, 1H, J=7.7 Hz), 8.10 (s, 1H), 8.82 (d, 1H, J=7.9 Hz); ¹³C NMR (125 MHz, DMSO-d₆) δ 56.94, 64.63, 111.34, 115.53, 126.66, 127.43, 127.56, 128.70, 130.97, 140.77, 141.20, 141.67, 146.67, 151.59, 161.26; MS (ESI) m/e (rel intensity): 372.3 (89), 374.2 (100).

### Example 17

### Spectral Characterization of a Compound of the Present Invention

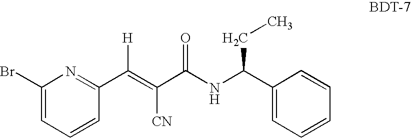

¹H NMR (500 MHz, CDCl₃) δ 0.95 (d, 3H, J=5.4 Hz), 1.94 (m, 1H), 4.99 (m 1H), 6.78 (d, 1H, J=4.8 Hz), 7.27-7.38 (m, 3H), 7.57 (d, 2H, J=4.7 Hz), 7.65 (dd, 1H, J=5.0, 4.3 Hz), 8.19 (s, 1H); ¹³C NMR (125 MHz, CDCl₃) δ 10.69, 29.09, 56.34, 109.54, 115.81, 125.41, 126.58, 127.76, 128.84, 130.64, 139.12, 141.01, 142.48, 148.26, 150.92, 158.67; MS (ESI) m/e (rel intensity): 370.1 (90), 372.1 (100).

### Example 18

### Spectral Characterization of a Compound of the Present Invention

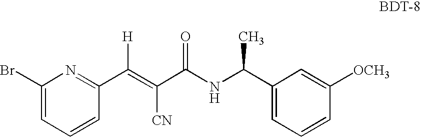

¹H NMR (500 MHz, CDCl₃) δ 1.62 (d, 3H, J=6.9 Hz), 3.84 (s, 3H), 5.23 (m 1H), 6.79 (d, 1H, J=7.6 Hz), 6.85 (dd, 1H, J=8.2, 2.5 Hz), 6.91 (m, 1H), 6.96 (d, 1H, J=8.0 Hz), 7.7.31 (t, 1H, J=7.9 Hz), 7.60 (m, 2H), 7.68 (t, 1H, J=7.8 Hz), 8.23 (s, 1H); ¹³C NMR (125 MHz, CDCl₃) δ 21.707, 50.23, 55.27, 109.47, 112.12, 113.03, 115.74, 118.30, 125.43, 129.97, 130.66, 139.14, 142.47, 143.68, 148.34, 150.91, 158.51, 159.99; MS (ESI) m/e (rel intensity): 386.2 (50), 388.2 (53).

### Example 19

### Spectral Characterization of a Compound of the Present Invention

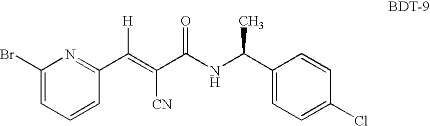

¹H NMR (500 MHz, CDCl₃) δ 1.59 (d, 3H, J=6.9 Hz), 5.20 (m 1H), 6.75 (d, 1H, J=6.5 Hz), 7.27 (d, 2H, J=8.6 Hz), 7.33 (d, 2H, J=8.6 Hz), 7.58 (m, 2H), 7.65 (m, 1H), 8.19 (s, 1H); ¹³C NMR (125 MHz, CDCl₃) δ 21.66, 49.70, 109.24, 115.72, 125.53, 127.56, 129.00, 130.76, 133.56, 139.15, 140.67, 142.50, 148.46, 150.80, 158.61; MS (ESI) m/e (rel intensity): 390 (70), 392 (85).

### Example 20

### Spectral Characterization of a Compound of the Present Invention

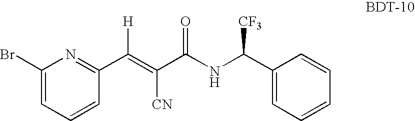

¹H NMR (300 MHz, CDCl₃) δ 5.80 (m, 1H), 7.16 (d, 1H, J=9.5 Hz), 7.45 (s, 5H), 7.61 (m, 2H), 7.69 (m, 1H), 8.25 (s, 1H); MS (ESI) m/e (rel intensity): 410.2 (94), 412.2 (100), 429.1 (28).

### Example 21

### Spectral Characterization of a Compound of the Present Invention

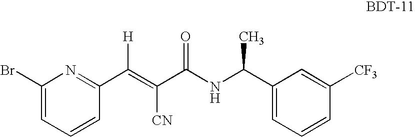

¹H NMR (500 MHz, CDCl₃) δ 1.66 (d, 3H, J=8.4 Hz), 5.31 (m 1H), 6.88 (d, 1H, J=8.4 Hz), 7.51 (m, 1H), 7.7.60 (m, 2H), 7.60 (m, 2H), 7.69 (m, 1H), 8.22 (s, 1H); ¹³C NMR (125 MHz, CDCl₃) δ 21.68, 49.94, 109.10, 115.70, 122.89, 122.99, 123.02, 124.62, 125.61, 129.37, 129.53, 130.80, 139.16, 142.50, 143.26, 148.60, 150.75, 158.74 MS (EI) m/e (rel intensity): 422.0 (53), 424.0 (67).

### Example 22

### Spectral Characterization of a Compound of the Present Invention

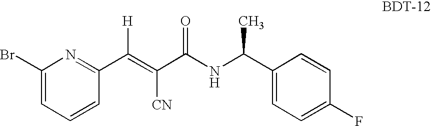

¹H NMR (500 MHz, CDCl₃) δ 1.62 (d, 3H, J=8.4 Hz), 5.24 (t 1H, J=8.4 Hz), 6.78 (d, 1H, J=8.4 Hz), 7.08 (m, 2H), 7.36 (m, 2H), 7.60 (m, 2H), 7.69 (m, 1H), 8.20 (s, 1H); ¹³C NMR (125 MHz, CDCl₃) δ 21.77, 49.70, 109.41, 115.70, 115.80, 115.87, 125.57, 127.91, 127.98, 130.80, 137.98, 139.22, 142.57, 148.49, 150.91, 158.62, 161.32, 163.28 MS (EI) m/e (rel intensity): 374.3 (11), 376.3 (11).

### Example 23

### Spectral Characterization of a Compound of the Present Invention

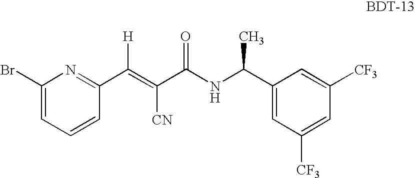

¹H NMR (500 MHz, CDCl₃) δ 1.67 (d, 3H, J=8.4 Hz), 5.31 (m 1H), 6.92 (d, 1H, J=8.4 Hz), 7.57 (m, 1H), 7.67 (m, 2H), 7.60 (m, 2H), 7.80 (s, 1H), 8.18 (s, 1H); ¹³C NMR (125 MHz, CDCl₃) δ 21.68, 49.94, 109.10, 115.70, 122.89, 122.99, 123.02, 124.62, 125.61, 129.37, 129.53, 130.80, 139.16, 142.50, 143.26, 148.60, 150.75, 158.74; MS (EI) m/e (rel intensity): 490.1 (86), 492.1 (100).

### Example 24

### Spectral Characterization of a Compound of the Present Invention

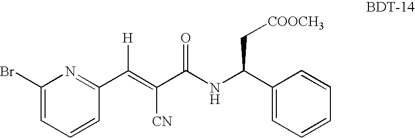

¹H NMR (300 MHz, CDCl₃) δ 2.98 (m, 2H), 3.70 s, 1H), 5.52 (m, 1H), 7.32 (m, 5H), 7.62 (m, 3H), 7.76 (d, 1H, J=7.8 Hz), 8.20 (s, 1H); ¹³C NMR (125 MHz, CDCl₃) δ 39.73, 50.82, 52.11, 109.48, 115.55, 125.39, 126.24, 128.09, 128.96, 130.70, 139.17, 139.56, 142.49, 148.51, 150.90, 158.84, 171.03.

### Example 25

### Spectral Characterization of a Compound of the Present Invention

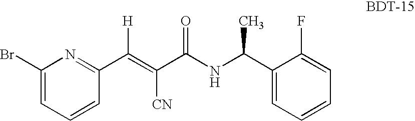

¹H NMR (500 MHz, CDCl₃) δ 1.61 (d, 3H, J=7.0 Hz), 5.41 (m 1H, J=8.4 Hz), 6.99 (d, 1H, J=7.0 Hz), 7.08 (m, 1H), 7.14 (t, 1H, J=7.5 Hz), 7.31 (m, 2H), 7.58 (m, 2H), 7.66 (m, 1H), 8.19 (s, 1H); ¹³C NMR (150 MHz, CDCl₃) δ 21.29, 46.53, 109.38, 115.65, 116.02, 116.16, 124.49, 124.51, 125.47, 127.84, 127.87, 129.07, 129.16, 129.37, 129.43, 130.68, 139.18, 142.45, 148.40, 150.88, 158.47, 159.82, 161.45.

### Example 26

### Spectral Characterization of a Compound of the Present Invention

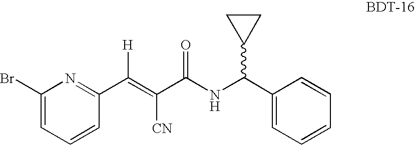

¹H NMR (600 MHz, CDCl₃) δ 0.44 (m, 1H), 0.50 (m, 1H), 0.68 (m, 2H), 1.29 (m, 1H), 4.51 (t, 1H, J=8.4 Hz), 6.99 (d, 1H, J=7.8 Hz), 7.30 (m, 1H), 7.38 (m, 4H), 7.58 (m, 2H), 7.67 (t, 1H, J=7.8 Hz), 8.20 (s, 1H); ¹³C NMR (600 MHz, CDCl₃) δ 4.20, 4.26, 16.69, 59.15, 107.83, 115.99, 126.71, 127.24, 127.94, 128.85, 130.97, 131.62, 133.05, 133.57, 140.66, 148.96, 158.22; MS (EI) m/e (rel intensity): 382.2 (60).

### Example 27

### Spectral Characterization of a Compound of the Present Invention

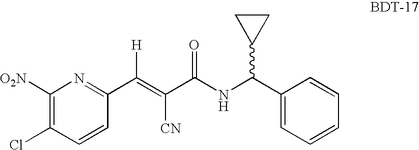

¹H NMR (600 MHz, CDCl₃) δ 0.44 (m, 1H), 0.51 (m, 1H), 0.68 (m, 2H), 1.30 (m, 1H), 4.50 (t, 1H, J=8.4 Hz), 6.80 (d, 1H, J=7.8 Hz), 7.31 (m, 1H), 7.36-7.40 (m, 4H), 7.69 (d, 1H, J=8.4 Hz), 8.08 (dd, 1H, J=8.4, 2.4 Hz), 8.28 (s, 1H), 8.33 (d, 1H, J=1.8 Hz); ¹³C NMR (600 MHz, CDCl₃) δ 4.20, 4.26, 16.69, 59.15, 107.83, 115.99, 126.71, 127.24, 127.94, 128.85, 130.97, 131.62, 133.05, 133.57, 140.66, 148.96, 158.22; MS (EI) m/e (rel intensity): 382.2 (60).

### Example 28

### Spectral Characterization of a Compound of the Present Invention

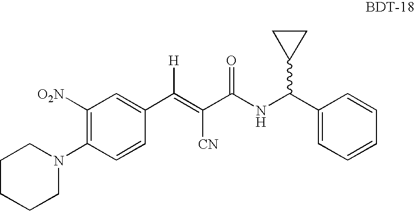

¹H NMR (600 MHz, CDCl₃) δ 0.43 (m, 1H), 0.51 (m, 1H), 0.66 (m, 2H), 1.27 (m, 1H), 1.58 (s, 2H), 1.68 (m, 2H), 1.72 (m, 4H), 3.22 (m, 4H), 4.50 (t, 1H, J=8.4 Hz), 6.72 (d, 1H, J=7.8 Hz), 7.10 (d, 1H, J=9.0 Hz), 7.29 (t, 1H, J=7.2 Hz), 7.35-7.40 (m, 4H), 8.08 (d, 1H, J=9.0 Hz), 8.15 (s, 1H), 8.24 (s, 1H); ¹³C NMR (600 MHz, CDCl₃) δ 4.11, 4.22, 16.77, 23.80, 25.64 51.87, 58.72, 101.13, 117.94, 119.97, 121.62, 126.73, 127.72, 128.74, 131.05, 134.16, 139.18, 141.16, 148.55, 150.54, 159.81; MS (ESI) m/e (rel intensity): 431.3 (100).

### Example 29

### Mass Spectral Characterization of a Compound of the Present Invention

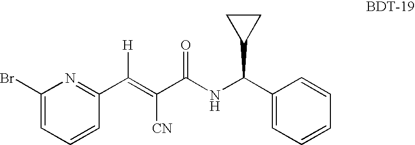

MS (EI) m/e (rel intensity): 382.3 (42), 384.3 (31), 399.2 (19), 401.2 (22), 414.3 (45).

### Example 30

### Spectral Characterization of a Compound of the Present Invention

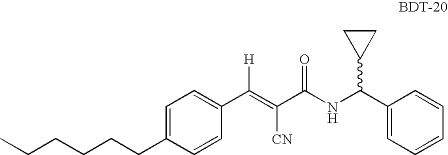

¹H NMR (600 MHz, CDCl₃) δ 0.44 (m, 1H), 0.51 (m, 1H), 0.66 (m, 2H), 0.91 (t, 1H, J=6.6 Hz), 1.26 (m, 1H), 1.35 (m, 4H), 1.46 (m, 2H), 1.80 (m, 2H), 4.03 (m, 2H), 4.51 (t, 1H, J=8.4 Hz), 6.73 (d, 1H, J=7.8 Hz), 6.96 (d, 1H, J=9.0 Hz), 7.29 (m, 1H), 7.35-7.41 (m, 4H), 7.91 (d, 1H, J=9.0 Hz), 8.24 (s, 1H).

### Example 31

### Spectral Characterization of a Compound of the Present Invention

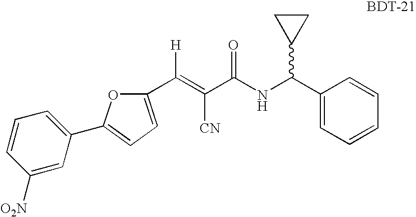

¹H NMR (600 MHz, CDCl₃) δ 0.44 (m, 1H), 0.52 (m, 1H), 0.68 (m, 2H), 1.28 (m, 1H), 4.52 (t, 1H, J=8.4 Hz), 6.80 (d, 1H, J=7.2 Hz), 7.05 (d, 1H, J=2.4 Hz), 7.20 (s, 1H), 7.30 (t, 1H, J=6.6 Hz), 7.39 (m, 4H), 7.68 (t, 1H, J=7.2 Hz), 8.06 (s, 1H), 8.19 (d, 1H, J=7.8 Hz), 8.23 (d, 1H, J=8.4 Hz), 8.63 (s, 1H); ¹³C NMR (600 MHz, CDCl₃) δ 4.03, 4.19, 16.74, 58.70, 100.54, 110.38, 112.54, 117.19, 119.87, 120.91, 123.32, 123.72, 126.65, 127.69, 128.70, 130.45, 130.50, 136.41, 141.03, 148.78, 149.03, 156.06, 159.53; MS (EI) m/e (rel intensity): 414.6 (100), 431.4 (42).

### Example 32

### Spectral Characterization of a Compound of the Present Invention

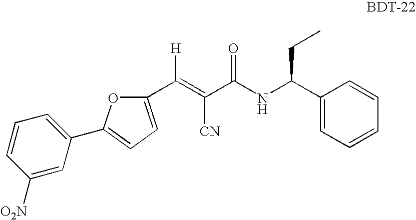

¹H NMR (600 MHz, CDCl₃) δ 0.96 (t, 1H, J=7.2 Hz), 1.94 (m, 2H), 4.99 (m, 1H), 6.57 (d, 1H, J=7.2 Hz), 7.04 (m, 1H), 7.19 (m, 1H), 7.33 (m, 5H), 7.68 (t, 1H, J=7.8 Hz), 8.05 (s, 1H), 8.18 (d, 1H, J=7.2 Hz), 8.22 (d, 1H, J=7.8 Hz), 8.63 (s, 1H).

### Example 33

### Spectral Characterization of a Compound of the Present Invention

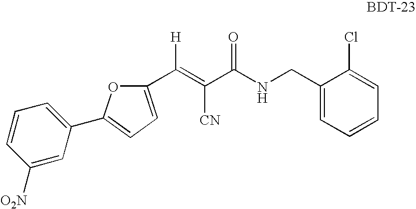

¹H NMR (600 MHz, CDCl₃) δ 3.17 (d, 1H, J=5.4 Hz), 4.50 (s, 2H), 7.36 (m, 3H), 7.46 (d, 1H, J=7.8 Hz), 7.52 (d, 1H, J=3.6 Hz), 7.65 (d, 1H, J=3.6 Hz), 7.84 (m, 1H), 8.09 (s, 1H), 8.26 (d, 1H, J=6.0 Hz), 8.31 (d, 1H, J=6.0 Hz), 8.72 (s, 1H), 8.96 (t, 1H, J=5.4 Hz).

### Example 34

### Spectral Characterization of a Compound of the Present Invention

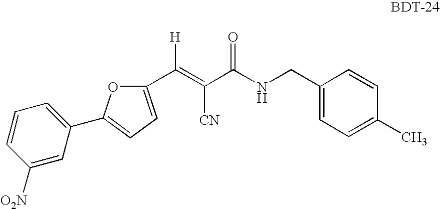

¹H NMR (600 MHz, DMSO-d₆) δ 2.28 (s, 3H), 3.17 (d, 1H, J=4.8 Hz), 4.37 (d, 1H, J=5.4 Hz), 7.14 (d, 2H, J=7.8 Hz), 7.20 (d, 2H, J=7.8 Hz), 7.49 (d, 1H, J=3.6 Hz), 7.63 (d, 1H, J=3.6 Hz), 7.84 (t, 1H, J=6.0 Hz), 8.06 (s, 1H), 8.25 (d, 1H, J=7.8 Hz), 8.30 (d, 1H, J=7.8 Hz), 8.71 (s, 1H), 8.91 (t, 1H, J=5.4 Hz).

### Example 35

### Spectral Characterization of a Compound of the Present Invention

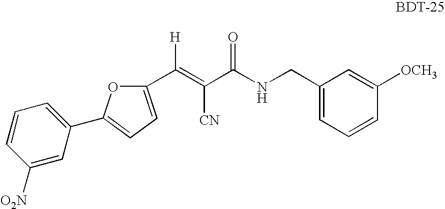

¹H NMR (600 MHz, DMSO-d₆) δ 3.75 (s, 3H), 4.40 (d, 1H, J=6.0 Hz), 6.83 (d, 1H, J=6.0 Hz), 6.89 (s, 2H), 7.26 (t, 1H, J=7.8 Hz), 7.50 (d, 1H, J=3.6 Hz), 7.63 (d, 1H, J=3.6 Hz), 7.84 (t, 1H, J=7.8 Hz), 8.07 (s, 1H), 8.25 (d, 1H, J=7.8 Hz), 8.31 (d, 1H, J=7.8 Hz), 8.72 (s, 1H), 8.94 (t, 1H, J=5.4 Hz).

### Example 36

### Spectral Characterization of a Compound of the Present Invention

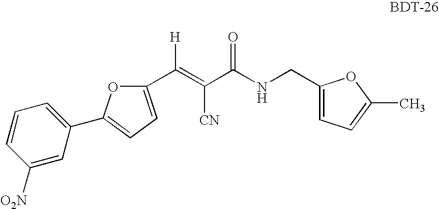

¹H NMR (600 MHz, DMSO-d₆) δ 2.24 (s, 3H), 4.35 (d, 2H, J=5.4 Hz), 6.00 (s, 1H), 6.16 (s, 1H), 7.49 (d, 1H, 3.6 Hz), 7.64 (d, 1H, J=3.6 Hz), 7.84 (t, 1H, J=7.8 Hz), 8.05 (s, 1H), 8.25 (d, 1H, J=8.4 Hz) 8.30 (d, 1H, J=8.4 Hz) 8.71 (s, 1H), 8.85 (t, 1H, J=5.4 Hz).

### Example 37

### Spectral Characterization of a Compound of the Present Invention

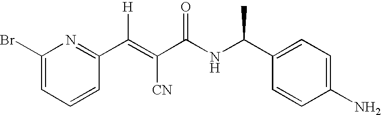

### MTAP-12k4

¹H NMR (600 MHz, DMSO-d₆) δ 8.72 (d, 1H, J=7.8 Hz), 8.57 (s, 1H), 8.15 (d, 1H, J=7.6 Hz), 7.91 (t, 1H, J=7.8 Hz), 7.79 (d, 1H, 7.8 Hz), 7.36 (m, 4H), 4.93 (p, 1H, J=7.2 Hz), 3.68 (s, 1H), 1.38 (d, 3H, J=7.0 Hz); ¹³C NMR δ 162.1, 159.5, 156.1, 149.5, 143.8, 142.1, 141.2, 130.7, 127.8, 122.3, 121.7, 117.1, 49.2, 26.3, 23.0; MS (C₁₇H₁₅BrN₄O) estimated 370.04 found, 371.2 and 373.2 (M+H).

### Example 38

### Spectral Characterization of a Compound of the Present Invention

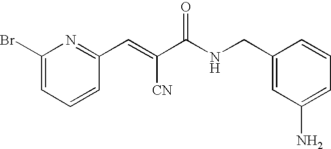

### MTAP-6

¹H NMR (600 MHz, DMSO-d₆) δ 8.74 (t, 1H, J=5.6 Hz), 8.57 (s, 1H), 7.15 (d, 1H, 10.1 Hz), 7.91 (t, 1H, J=7.8 Hz), 7.84 (d, 1H, J=7.8 Hz), 7.35 (s, 4H), 4.32 (d, 2H, J=5.8 Hz), 3.7 (s, 2H); MS (C₁₆H₁₃BrN₄O) estimated 356.03 found, 355.3 and 357.3 (M+H).

### Example 39

### Spectral Characterization of a Compound of the Present Invention

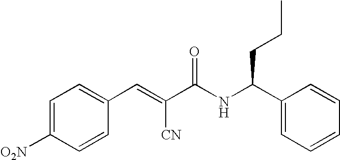

### MTAP-11

¹H NMR (600 MHz, CDCl₃) δ 9.08 (d, 1H, 2.1 Hz), 8.73 (s, 1H), 8.60 (dd, 1H, J=6.0 and J=2.1 Hz), 7.32 (m, 5H), 6.6 (d, 2H, J=8.1 Hz), 5.08 (q, 1H, J=7.5 Hz), 1.90 (m, 2H), 1.46 (m, 2H), 0.96 (t, 3H, J=7.5 Hz); ¹³C NMR δ 157.1, 151.2, 148.7, 140.9, 134.4, 132.2, 128.9, 128.9, 128.5, 127.9, 126.6, 126.6, 120.9, 115.4, 110.0, 54.9, 38.0, 19.5, 13.7; MS (C₂₀H₁₉N₃O₃) estimated 349.14 found, 350.2 (M+H).

### Example 40

### Spectral Characterization of a Compound of the Present Invention

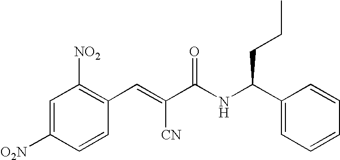

### MTAP-12

¹H NMR (600 MHz, CDCl₃) δ 9.11 (d, 1H, 1.0 Hz), 8.76 (s, 1H), 8.64 (dd, 1H, J=4.5 and J=1.2 Hz), 7.98 (d, 1H, 4.2 Hz), 7.35 (m, 5H), 6.58 (d, 1H, J=3.9 Hz), 5.10 (q, 1H, J=7.5 Hz), 1.93 (m, 2H), 1.39 (m, 2H), 0.99 (t, 3H, J=7.5 Hz); ¹³C NMR δ 157.1, 149.1, 148.7, 140.9, 134.4, 132.0, 128.9, 128.9, 128.5, 128.0, 127.9, 126.6, 126.6, 120.9, 114.6, 112.3, 54.9, 38.0, 19.5, 13.7; MS (C₂₀H₁₈N₄O₅) estimated 394.12 found, 393.2 (M−H).

### Example 41

### Spectral Characterization of a Compound of the Present Invention

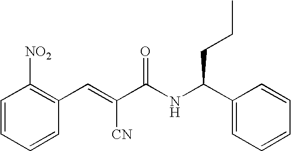

### MTAP-13

¹H NMR (600 MHz, CDCl₃) δ 8.71 (d, 1H, 2.0 Hz), 8.23 (dd, 1H, J=4.5 and J=1.2

Hz), 7.75 (d, 2H, 4.2 Hz), 7.65 (m, 1H), 7.35 (m, 4H), 7.29 (m, 1H), 6.68 (m, 1H), 5.09 (q, 1H, J=7.5 Hz), 1.89 (m, 2H), 1.37 (m, 2H), 0.93 (t, 3H, J=7.5 Hz); ¹³C NMR δ 158.1, 151.2, 147.5, 141.3, 134.4, 131.8, 130.4, 128.8, 128.8, 128.7, 127.7, 126.7, 126.7, 125.4, 115.4, 110.0, 54.7, 38.1, 19.5, 13.8; MS (C₂₀H₁₉N₃O₃) estimated 349.14 found, 348.5 (M−H).

### Example 42

### Spectral Characterization of a Compound of the Present Invention

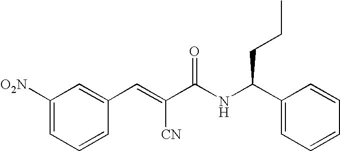

### MTAP-14

¹H NMR (600 MHz CDCl₃) δ 8.68 (d, 1H, 1.8 Hz), 8.36 (m, 2H), 8.24 (d, 1H, 7.8 Hz), 7.70 (t, 1H, J=7.8 Hz), 7.33 (m, 5H), 6.62 (d, 1H, J=7.8 Hz), 5.08 (q, 1H, J=7.2 Hz), 1.90 (m, 2H), 1.38 (m, 2H), 0.96 (t, 3H, J=7.2 Hz); ¹³C NMR δ 158.3, 150.1, 148.7, 141.2, 135.1, 133.4, 130.4, 128.9, 128.9, 127.8, 126.6, 126.5, 125.2, 116.0, 107.7, 54.8, 38.1, 19.5, 13.7; MS (C₂₀H₁₉N₃O₃) estimated 349.14 found, 348.3 (M−H).

### Example 43

### Spectral Characterization of a Compound of the Present Invention

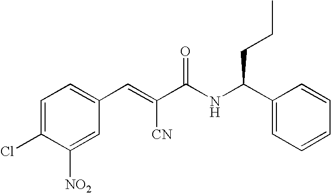

### MTAP-15

¹H NMR (600 MHz, CDCl₃) δ 8.32 (d, 1H, 2.4 Hz), 8.26 (s, 1H), 8.07 (dd, 1H, 6.0 and 1.8 Hz), 7.68 (d, 1H, J=8.4 Hz), 7.31 (m, 5H), 6.58 (d, 1H, J=7.8 Hz), 5.08 (q, 1H, J=7.2 Hz), 1.90 (m, 2H), 1.38 (m, 2H), 0.96 (t, 3H, J=7.2 Hz); ¹³C NMR δ 158.1, 148.7 147.5, 141.1, 133.5, 132.9, 131.6, 130.8, 128.9, 128.9, 127.8, 127.1 126.5, 126.5, 115.9, 107.8, 54.8, 38.1, 19.5, 13.7; MS (C₂₀H₁₈ClN₃O₃) estimated 383.10 found, 384.2 (M+H).

### Example 44

### Spectral Characterization of a Compound of the Present Invention

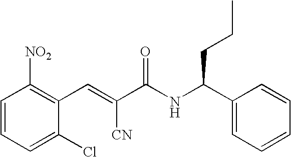

### MTAP-16

¹H NMR (600 MHz, CDCl₃) δ 8.54 (s, 1H), 8.16 (d, 1H, J=8.4 Hz), 7.82 (d, 1H, 7.8 Hz), 7.59 (t, 1H, J=8.4 Hz), 7.36 (m, 5H), 6.62 (d, 1H, J=7.8 Hz), 5.10 (q, 1H, J=7.2 Hz), 1.90 (m, 2H), 1.36 (m, 2H), 0.97 (t, 3H, J=7.2 Hz); ¹³C NMR δ 157.4, 149.0, 148.3, 141.1, 135.5, 134.7, 131.1, 128.9, 128.9, 128.0, 127.8, 126.7, 126.7, 123.8, 114.2, 113.5, 54.8, 38.1, 19.5, 13.8; MS (C₂₀H₁₈ClN₃O₃) estimated 383.10 found, 382.2 (M−H).

### Example 45

### Spectral Characterization of a Compound of the Present Invention

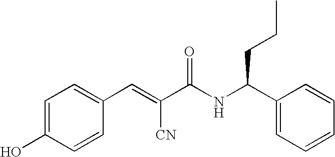

### MTAP-17

¹H NMR (600 MHz, CDCl₃) δ 8.14 (s, 1H), 7.86 (dd, 2H, J=6.0 and J=2.1 Hz), 7.32 (m, 5H), 6.7 (d, 2H, J=8.1 Hz), 6.62 (d, 1H, J=7.8 Hz), 5.08 (q, 1H, J=7.2 Hz), 1.90 (m, 2H), 1.46 (m, 2H), 0.96 (t, 3H, J=7.5 Hz); ¹³C NMR δ 160.9, 157.0, 155.7, 151.2, 144.1, 135.2 132.0, 131.4, 128.9, 128.9, 127.7, 126.5, 126.5, 125.9, 117.0, 111.1, 54.8, 38.3, 19.5, 13.7; MS (C₂₀H₂₀N₂O₂) estimated 320.15 found, 321.6 (M+H).

### Example 46

### Spectral Characterization of a Compound of the Present Invention

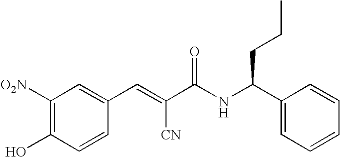

### MTAP-18

¹H NMR (600 MHz, CDCl₃) δ 8.25 (s, 1H), 7.20 (d, 1H, J=9.0 Hz), 7.60 (s, 1H), 7.49 (d, 1H, J=8.4 Hz), 7.33 (m, 5H), 6.65 (d, 1H, J=7.8 Hz), 5.07 (q, 1H, J=7.2 Hz), 1.90 (m, 2H), 1.44 (m, 2H), 0.97 (t, 3H, J=7.5 Hz); ¹³C NMR δ 158.1, 154.9, 149.8 141.1, 139.9, 134.8, 132.9, 131.6, 130.8, 128.9, 128.9, 127.8, 126.5, 126.5, 126.0, 121.9, 120.9, 115.8, 109.2, 54.8, 38.1, 19.5, 13.7; MS (C₂₀H₁₉N₃O₄) estimated 365.137 found, 366.5 (M+H).

### Example 47

### Spectral Characterization of a Compound of the Present Invention

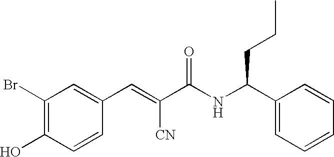

### MTAP-19

¹H NMR (600 MHz CDCl₃) δ 8.13 (s, 1H), 8.03 (s, 1H), 7.20 (d, 1H, J=8.4 Hz), 7.30 (m, 5H), 7.05 (dd, 1H, J=8.4 and 0.6 Hz), 6.59 (d, 1H, J=7.8 Hz), 5.07 (q, 1H, J=7.2 Hz), 1.90 (m, 2H), 1.44 (m, 2H), 0.97 (t, 3H, J=7.5 Hz); ¹³C NMR δ 156.6, 153.6, 141.1, 137.7, 132.9, 130.6, 130.4, 130.4, 128.9, 127.8, 126.5, 126.5, 123.5, 123.5, 122.3, 122.2, 120.6, 116.9, 114.8 54.9, 38.2, 19.5, 13.7; MS (C₂₀H₁₉BrN₂O₂) estimated 398.06 found, 399.4 and 401.2 (M+H)

### Example 48

### Spectral Characterization of a Compound of the Present Invention

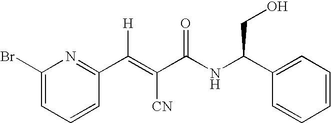

### MTAP-20

2-Propenamide, 2-cyano-3-(3-bromo 2-pyridinyl)-N-(1-phenyl-1-ethylhydroxyl)-(E) ¹H NMR (600 MHz, CDCl₃) δ 8.21 (s, 1H), 7.68 (t, 1H, J=7.8 Hz), 7.60 (m, 2H), 7.35 (m, 5H), 7.01 (d, 2H, J=8.4 Hz), 5.22 (p, 1H, J=7.2 Hz), 4.00 (m, 2H), 2.13 (bs, 1H); ¹³C NMR δ 159.6, 150.8, 148.6, 142.45, 139.2, 137.9, 130.8, 129.1, 127.7, 128.2, 126.7, 126.7, 125.5, 115.7, 109.3, 65.9, 56.5; MS (C₁₇H₁₄BrN₃O₂) estimated 371.02 found, 372.3 and 374.2 (M+H).

### Example 49

### Spectral Characterization of a Compound of the Present Invention

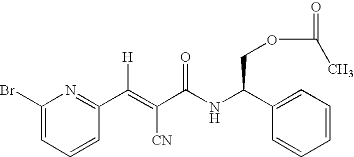

### MTAP-22

¹H NMR (600 MHz, CDCl₃) δ 8.20 (s, 1H), 7.67 (m, 1H), 7.59 (m, 2H), 7.38 (m, 5H), 7.28 (d, 1H, J=8.4 Hz), 6.50 (d, 1H, J=7.2 Hz), 5.39 (m 1H), 4.44 (m 2H), 2.08 (s, 3H); ¹³C NMR δ 171.0, 159.1, 150.7, 148.6, 142.5, 139.2, 137.3, 130.8, 129.0, 129.0, 128.4, 126.7, 126.7, 125.6, 115.6, 109.2, 65.8, 53.7, 20.8; MS (C₁₉H₁₆BrN₃O₃) estimated 413.037 found, 414.1 and 416.1 (M+H).

### Example 50

### Spectral Characterization of a Compound of the Present Invention

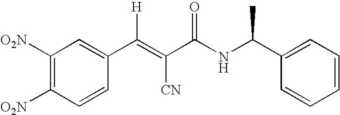

### MTAP-23

¹H NMR (600 MHz, CDCl₃) δ 8.23 (s, 1H), 8.16 (s, 1H), 8.08 (d, 1H, J=9.0 Hz), 7.37 (m, 5H), 7.10 (d, 1H, J=9.0 Hz), 5.23 (p, 1H, J=7.2 Hz), 1.59 (d, 3H, J=7.2 Hz; MS (C₁₈H₁₄N₄O₅) estimated 366.096 found, 367.2 (M+H).

### Example 51

### Spectral Characterization of a Compound of the Present Invention

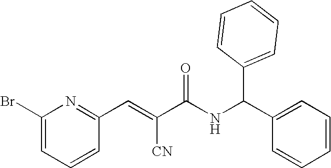

### MTAP-24

¹H NMR (600 MHz, CDCl₃) δ 8.26 (s, 1H), 7.67 (m, 1H), 7.59 (m, 2H), 7.38 (m, 5H), 7.33 (m, 11H), 6.50 (d, 1H, J=7.2 Hz); ¹³C NMR δ 158.8, 150.8, 148.8, 142.5, 140.3, 139.2, 130.8, 129.1, 129.0 (4C), 128.3 (2C), 128.0, 127.4 (4C), 126.9, 125.7, 115.7, 109.1, 58.2; MS (C₂₂H₁₆BrN₃O) estimated 417.047 found, 418.1 and 420.1 (M+H).

### Example 52

### Spectral Characterization of a Compound of the Present Invention

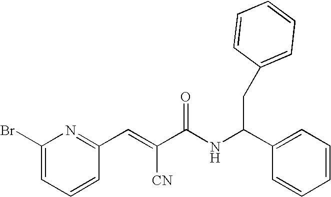

### MTAP-25

¹H NMR (600 MHz, CDCl₃) δ 8.06 (s, 1H), 7.56 (t, 1H, J=7.8 Hz), 7.50 (d, 1H, J=7.8 Hz), 7.45 (d, 1H, J=7.8 Hz), 7.20 (m, 10H), 5.34 (q, 1H, J=7.2 Hz), 3.19 (m, 2H), ¹³C NMR δ 158.8, 150.8, 148.3, 142.4, 140.8, 139.3, 136.8, 130.7, 129.4 (2C), 128.8 (2C), 128.7 (2C), 127.8, 126.9, 126.6 (2C), 125.8, 115.7, 109.1, 56.0, 42.5; MS (C₂₃H₁₈BrN₃O) estimated 431.063 found, 432.2 and 434.2 (M+H).

### Example 53

### Spectral Characterization of a Compound of the Present Invention

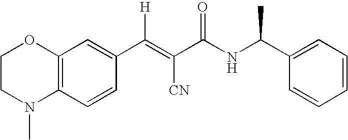

### MTAP-26

¹H NMR (600 MHz, CDCl₃ δ 8.06 (s, 1H), 7.47 (d, 1H, J=1.2 Hz), 7.42 (dd, 1H, J=9.0 and 2.4 Hz), 7.33 (m, 4H), 7.27 (m, 1H), 6.60 (d, 1H, 8.4 Hz), 6.51 (d, 1H, J=6.6 Hz), 5.20 (p, 1H, J=7.2 Hz), 4.22 (t, 2H, J=4.8 Hz), 3.41 (t, 2H, J=4.8 Hz), 2.99 (s, 3H), 1.56 (d, 3H, J=6.6 Hz); ¹³C NMR δ 160.4, 152.7, 143.2, 142.8, 131.7, 128.8 (2C), 128.2, 127.6, 126.2, 126.1 (2C), 120.9, 118.6, 117.2, 110.8, 63.8, 49.9, 48.7, 38.1, 21.5; MS (C₂₁H2₁N₃O₂) estimated 347.163 found, 348.3 (M+H).

### Example 54

### Spectral Characterization of a Compound of the Present Invention

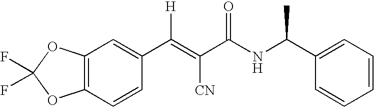

### MTAP-27

¹H NMR (600 MHz, CDCl₃) δ 8.26 (s, 1H), 7.79 (d, 1H, J=1.2 Hz), 7.82 (d, 1H, 7.8 Hz), 7.58 (dd, 1H, J=8.4 and 1.8 Hz), 7.36 (m, 4H), 7.30 (m, 1H), 7.17 (d, 1H, 8.4 Hz), 6.56 (d, 1H, J=6.6 Hz), 5.24 (p, 1H, J=7.2 Hz), 1.60 (d, 3H, J=6.6 Hz); ¹³C NMR δ 158.9, 151.5, 146.6, 144.4, 142.1, 131.7, 128.9 (4C), 128.1, 127.9, 126.2 (2C), 116.7, 110.1, 103.9, 50.3, 21.7; MS (C₁₉H₁₄F₂N₂O₃) estimated 356.097 found, 358.1 (M+H).

### Example 55

### Spectral Characterization of a Compound of the Present Invention

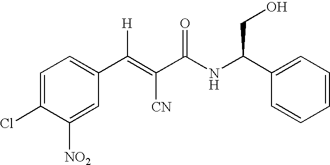

### MTAP-28

¹H NMR (600 MHz, DMSO-d₆) δ 8.32 (d, 1H, J=8.4 Hz), 8.13 (s, 1H), 8.08 (dd, 1H, J=9.0 and 2.4 Hz), 7.36 (m, 4H), 7.30 (m, 1H), 7.08 (d, 1H, 8.4 Hz), 7.04 (d, 1H, J=6.6 Hz), 5.20 (q, 1H, J=7.2 Hz), 3.96 (m, 2H), 2.08 (s, 3H); ¹³C NMR δ 160.9, 150.7, 148.6, 139.0, 138.3, 134.1, 131.2, 130.8, 129.0, 129.0, 128.2, 126.7, 126.7, 125.6, 121.4, 119.9, 117.3, 100.8, 65.9, 56.5, 20.8; MS (C₁₈H₁₄ClN₃O₄) estimated 371.067 found, 372.2 (M+H).

### Example 56

### Spectral Characterization of a Compound of the Present Invention

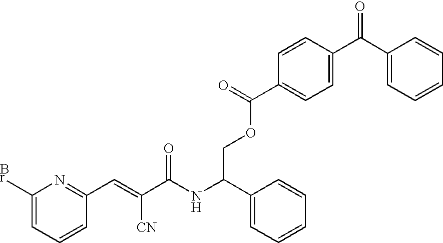

### MTAP-29

¹H NMR (600 MHz, CDCl₃) δ 8.22 (s, 1H), 8.15 (d, 2H, J=8.4 Hz), 7.85 (d, 2H, 7.8 Hz), 7.81 (m, 2H), 7.65 (m, 4H), 7.52 (t, 2H, J=7.8 Hz), 7.45 (m, 4H), 7.38 (m, 2H), 5.60 (m, 1H), 4.74 (m, 2H); ¹³C NMR δ 197.0, 159.2, 150.7, 148.6, 142.5, 141.7, 139.2, 137.1, 136.9, 133.0, 130.8, 130.1 (2C), 129.9 (2C), 129.7 (2C), 129.2 (2C), 128.6, 128.5 (2C), 126.7 (2C), 125.6, 119.9, 117.3, 109.1, 66.7, 53.9; MS (C₃₁H₂₂BrN₃O₄) estimated 579.079 found, 580.3 and 582.3 (M+H).

### Example 56

### Methodology for Assessing Biological Effects and Activity of WP, BDT, MTAP and CP Compounds

FIGS. 1-31 depict various exemplary data gathered from this Example.

### A. Screening for Anti-Tumor Activity.

All compounds are tested for purity (HPLC, NMR) and the calculated molecular weight is used to make up a 10 mM stock solution of each compound (in 100% dimethylsulfoxide or 50% dimethylsulfoxide:50% polyethylene glycol 300). Compounds are diluted into cell culture media consisting of RPMI 1640 with 10% fetal bovine serum to a final starting concentration of 10 μM. [Highest final DMSO content=0.1%.]. Tumor cells (20,000 for non-adherent cells; 5,000 for adherent cells) are plated into individual wells of a 96-well culture plate in 0.1 ml of culture media. Diluted compounds are added to individual wells containing pre-plated cells (final volume 0.2 ml) and incubated at 37° C. for 24 to 72 h. Wells receiving vehicle alone acted as a control.

MTT (viability) assay. MTT reagent (20 μl of 5 mg/ml stock solution, Sigma) is added to the cells and the plates are incubated at 37° C. for another 2 h. Cells are lysed by adding 100 μl of lysis buffer (20% SDS in 50% N,N-dimethylformamide (Sigma) adjusted to pH 4.7 by 80% acetic acid and 1 M HCl such that the final concentration of acetic acid is 2.5% and HCl is 2.5%) into each well and incubated for 6 h. The OD₅₇₀ of each sample is determined by using a SPECTRA MAX M2 plate reader (Molecular Devices). The OD in control and treated wells is used as an estimate of the effect of compounds on cell growth and survival.

Cell lines. B-cell malignancies [multiple myeloma—MM-1, OPM-1 and OPM-2; Mantle cell lymphoma—Mino, Non-Hodgkin's lymphoma—LP], chronic myelogenous leukemia (CML) [K562 (cell line derived from a patient with CML erythroid blast crisis), K562R (a clonal variant of K562 cells resistant to imatinib and overexpresses Lyn kinase), BV173 (cell line derived from a patient with CML lymphoblastic crisis), BV173R (a clonal imatinib resistant variant of BV173 that expresses T315I mutant Bcr/Abl) were grown in RPMI 1640 containing 10% heat-inactivated fetal bovine serum and 2 mM glutamine. A375 melanoma cells were grown and maintained in the same media. Ba/F3 parental cells were grown in RPMI 1640 containing 10% heat inactivated fetal bovine serum and 2 mM glutamine supplemented with IL-3 (1 ng/ml) while Ba/F3 cells stably expressing Bcr/Abl or the T315I mutant of Bcr/Abl were grown in RPMI 1640 containing 10% heat inactivated fetal bovine serum and 2 mM glutamine in the absence of IL-3. Normal human dermal fibroblasts (NHDF) were obtained from Cambrex (Walkersville, Md.) and grown in specially formulated media (obtained through Cambrex) with 20% fetal calf serum.

Generation of the Ba/F3 Bcr/Abl T315I mutant expressing stable cell line. Ba/F3 cells growing in RPMI medium supplemented with 10% FCS and IL-3 (1 ng/ml) were harvested, washed in 1×PBS and 2×10⁶ cells and transfected with cDNA representing wild type Bcr/Abl (pSG-Bcr/Abl) or the Bcr/Abl T315I mutant (introduced by site-directed mutagenesis using the Stratagene Quickchange II XL kit and confirmed by direct sequencing). DNA (5 μg) was electroporated (Amaxa Systems, solution T, 017 setting) into Ba/F3 cells that were incubated in 2 ml of RPMI medium supplemented with 10% FCS and IL-3 (1 ng/ml) for 24 h. Transfected cells were then washed in PBS and further incubated in RPMI medium supplemented with 10% FCS but lacking IL-3. Viable colonies that had been cultured in IL-3 negative medium for 4 weeks were screened for the expression of Bcr/Abl by Western blot. Expression of the T315I mutant was confirmed by loss of imatinib-mediated apoptosis and Bcr/Abl kinase inhibition (immunoblotting) in cell transfectants.

Trypan Blue Exclusion (Proliferation) Assay. Control or treated cells were harvested by centrifugation at 1200 rpm for 5 min and the resulting pellet was washed in PBS, centrifuged a second time and the resulting cell pellet was resuspended in 1 ml of medium. Viable cells were counted using a hemacytometer following a 5 min incubation of cells in trypan blue dye.

Quantitation of apoptosis. Hypodiploidy was measured in treated and control cells by propidium iodide (PI) staining and fluorescence-activated cell sorting (FACS). For these experiments, cells were seeded in six-well plates at 2×10⁶ cells per well for 1 day prior to treatment with compound. Cells were harvested at 0, 24, 48 and 72 h after treatment, washed twice in PBS, resuspended in 420 μl of PBS, then 980 μl of cold 100% ethanol was added drop-wise into each tube while the tubes were being vortexed at slow speed. The ethanol-fixed cells were stored at −20° C. until analyzed. Fixed cells were centrifuged between 7,000 rpm and 8,000 rpm for 5 min after which the pellet was resuspended in 500 μl of PBS/RNAse (final concentration, 0.1 mg/ml). The cells were incubated at 37° C. for 15 min, mixed with 500 μl of PBS containing PI at a final concentration of 25 μg/ml, and analyzed by FACScan cytofluorometer (Becton Dickinson, San Jose, Calif.).

Anti-tumor studies. Swiss Nu/Nu mice were obtained from the breeding facility in the Department of Experimental Radiotherapy at M.D. Anderson Cancer Center. On Day 0, 200 μl of an A375 cell suspension (5×10⁶ cells/ml) were injected (s.c.) into female Swiss nude mice 6-7 weeks of age. At the interval noted, compounds were injected (i.p. or oral gavage) into tumor bearing mice in a 0.1 ml suspension of DMSO/PEG300 (50/50). Degrasyn (40 mg/kg) was administrated on an every other day schedule, for 5 or more injections. In some experiments, imatinib (50 mg/kg) was administrated on a qd, 5 days on/2 days off schedule, for 1.5 weeks. Five to 8 mice per experimental group were used, including vehicle (DMSO/PEG300) control group. Tumor volumes were measured every other day using calipers (Cel Associates, Houston, Tex.).

### B. Assessment of Biological Activity

Antibodies. Primary antibodies—Anti-phosphotyrosine antibody (clone 4G10: Upstate Biotechnology, Lake Placid, N.Y.), Anti-pStat3, Anti-STAT5, Anti-pSTAT5, anti-CrkL, anti-pCrkL anti-HSP90, anti-HSP70, phosphorylated p38 and JNK/SAPK and anti-bcr antibodies (Cell Signaling, Danvers, Mass.), anti-pSTAT5 A/B (Tyr 694/699), anti-HA, anti-Actin (Sigma, St. Louis, Mo.), anti-HCK, anti-pHCK, anti-Stat3, anti-c-myc and anti-Jak2 (conjugated to agarose beads and free antibody) [Santa Cruz Biotechnology, Santa Cruz, Calif.] and anti-Abl 8E-9 (Pharmingen, San Diego, Calif.). MAPK and pMAPK (Promega, Madison, Wis.), Akt and pAkt (New England Biolabs, Beverly, Mass.).

Secondary antibodies—Peroxidase-conjugated affiniPure Goat Anti-Mouse and Anti-Rabbit antibodies (Jackson ImmunoResearch Laboratories, West Grove, Pa.)

Western Blot analysis. Cells were harvested, washed twice in cold 1×PBS and lysed in modified RIPA lysis buffer (150 mM NaCl, 1% NP-40, 0.5% Na-deoxycholate [DOC], 0.1% Na-dodecyl sulfate [SDS], 50 mM Tris-HCl (pH 7.5), 1 mM EDTA, 1 mM NaF, 2 mM Na-vanadate, with protease inhibitors (1 mM PMSF, 1 mM benzamidine, 10 μg/ml aprotinin, 10 μg/ml leupeptin) on ice for 30 min. The lysed cells were centrifuged at 13,000 rpm for 30 min and the supernatant collected. The protein concentration of the cell lysate was determined by the Bradford Protein Assay. Fifty to 60 μg of protein were resolved in a 10% SDS-PAGE, and transferred to a PVDF membrane. Western blotting was performed using specific primary antibodies Peroxidase-conjugated affiniPure anti-Mouse Anti-Rabbit secondary antibodies (Jackson Immunoresearch Laboratories, West Grove, Pa.). The proteins were visualized with ECL Plus reagents (Amersham Biosciences).

Real-Time Quantitative TR-PCR analysis of bcr/abl mRNA. Total RNA was isolated by lysing 5−10×10⁶ cells using 1 ml TRIzol Reagent (Invitrogen, Carlsbad, Calif.) for 5 min at room temperature. 200 μl of chloroform were added and well-mixed into the cell lysate for 30 sec and the mixture centrifuged at 12,000 rpm for 15 min. The supernatant was transferred into a fresh tube and the RNA precipitated by the addition of 500 μl isopropanol, mixed well for 5 min at room temperature and centrifuged at 12,000 rpm for 15 min. The pellet was washed in 75% alcohol in DEPC water and the sample centrifuged at 12,000 rpm for 15 min. The washed pellet was air-dried and resuspended in 30-50 μl RNase free water. cDNA was synthesized from 1 μg of RNA using the iScript™cDNA Synthesis Kit (Bio-Rad, Hercules, Calif.) following the instructions of the manufacturer. Real-time PCR was performed using the iCycleriQ thermocycler from Bio-Rad. Ubiquitin primers, reverse primers and a probe as well as the p210 bcr/abl (b3a2-1) forward primer, reverse primer and a probe were used in the reactions. All primers were synthesized by Sigma (St. Louis, Mo.) and the probes were synthesized by Bio-Rad.

Isolation of cellular fractions from clinical specimens. Mononuclear cells were isolated from peripheral blood or bone marrow of imatinib-resistant CML patients after informed consent was obtained for a protocol reviewed and approved by the Institutional Review Board of M.D. Anderson Cancer Center. Cells were purified on Ficoll-Hypaque gradients.

Analysis of wild-type and mutant (deleted) c-myc protein stability. A c-myc expression vector (pCGN-MYC) was obtained from Dr. William Tansey (Cold Springs Harbor, N.Y.) and initially used to transfect HeLa cancer cells (with the SN2 cationic lipid). In subsequent studies, the c-myc gene was subcloned downstream of the HA antigen in the pcDNA3.1 vector, allowing efficient detection of transfected c-myc by anti-HA immunoblotting. Multiple site-specific mutations and domain deletions were introduced into the c-myc gene using the Stratagene Quickchange II XL kit and specific dual primer sets. The deletions/mutations were verified by direct sequencing before use. HeLa cells were transfected with the amount of vector DNA indicated and incubated overnight before treatment of cells with degrasyn for brief intervals (5-120 min; as indicated). Cell lysates were prepared and immunoblotted with anti-HA for c-myc detection.

IC50 determinations. The anti-tumor activity of certain compounds of the present invention against MM-1 cell lines was investigated and the IC50 values calculated (FIGS. 33A-F). In a typical experiment, MM-1 cell lines were incubated with a range of compound concentrations for 72 h to determine the concentration required to inhibit cell growth or induce apoptosis by 50% (IC50). MTT assays as described above were used to conduct this analysis.

### REFERENCES

The following references, to the extent that they provide exemplary procedural or other details supplementary to those set forth herein, are specifically incorporated herein by reference.
U.S. Pat. No. 6,596,828
U.S. Pat. No. 6,433,018
U.S. Pat. No. 6,420,338
U.S. Application Ser. No. 60/806,426
U.S. Application Ser. No. 60/826,052
U.S. Application Publ. No. 2003/0013748
U.S. Application Publ. No. 2005/0277680
U.S. application Ser. No. 11/695,547
PCT International Pub. No. WO 1995/028922
PCT International Pub. No. WO 2005/058829
Alas and Bonavida, Clin. Cancer Res. 9(1):316-26, 2003.
Arbel et al., Am. J. Obstet. Gynecol. 188(5):1283-90, 2003.
Barton et al., Mol Cancer Ther. 3:11-20, 2004.
Bharti et al., J. Immunol., 171(7):3863-3871, 2003.
Bundgaard, H., Design of Prodrugs, pp. 7-9, 21-24, Elsevier, Amsterdam 1985.
Bundgaard, Drugs of the Future, 16:443-458, 1991.
Burdelya et al., Mol. Cancer. Ther. 1(11):893-9, 2002.
Catlett-Falcone et al., Immunity 10(1):105-15, 1999.
Constantin et al., Eur. J. Immunol. 28(11):3523-9, 1998.
De Vos et al., Br J Haematol. 109:823-8, 2000.
Epling-Burnette et al., J Clin Invest. 107:351-62, 2001.
Garcia et al., Oncogene 20:2499-513, 2001.
Gu, et al., Bioorg. Med. Chem. 13:4269-4278, 2005.
Hallek et al., Blood 91(1):3-21, 1998.
Handbook of Pharmaceutical Salts: Properties, Selection and Use (P. H. Stahl & C.
G. Wermuth eds., Verlag Helvetica Chimica Acta, 2002.
Hideshima et al., J. Biol. Chem. 277(19):16639-47, 2002.
Jernberg-Wiklund et al., Int. J. Cancer 51(1):116-23, 1992.
Kerr et al., FEBS Lett. 546(1):1-5, 2003.
Kuehl et al., Curr. Top Microbiol. Immunol. 224:277-82, 1997.
Lai et al., Oncogene 24:4442-9, 2005. Erratum in: Oncogene 24:8216, 2005.
Lee et al., Mol Cancer Ther. 5:138-48, 2006.
Liu et al., Am J Respir Cell Mol. Biol. 27:306-13, 2002.
Meydan et al., Nature 379(6566):645-8, 1996.
Nielsen et al., Proc Natl Acad Sci USA. 94:6764-9, 1997.
Niu et al., Oncogene 21:7001-10, 2002.
Remington's Pharmaceutical Sciences 15th Edition, pages 1035-1038 and 1570-1580.
Selvanayagam et al., Blood 71(1):30-5, 1988.
Shoulda et al., Cancer Lett. 23:176-84, 2006.
Toyonaga et al. Cancer Lett. 201:107-16, 2003.
Verma et al., Cancer Metastasis Rev. 22(4):423-34, 2003.

1. A compound selected from the group consisting of:
(a) compounds of the formula: 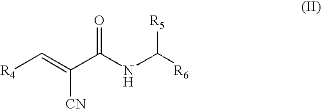
wherein:
R₄ is furanyl, thienyl, indolyl, ortho-bromopyridyl, 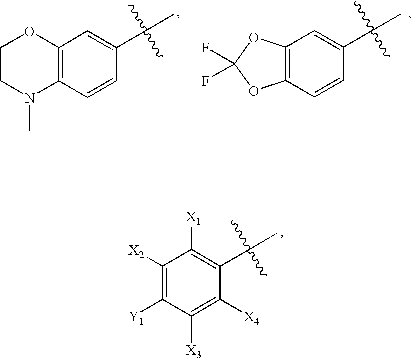
wherein:
X₁, X₂, X₃ and X₄ are each independently H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, halo, hydroxy, amino, azido, mercapto, nitro, or cyano; and
Y₁ is H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, amino, azido, mercapto, or cyano; or 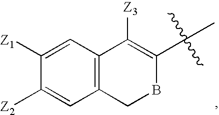
wherein
Z₁ is H or OH;
Z₂ is H, chloro, or —OCH₃;
Z₃ is H or chloro; and
B is C or O;
R₅ is H, alkyl, phenyl, or biotinyl; and
R₆ is phenyl or methylfuranyl;
provided that if R₄ is ortho-bromopyridyl, then R₅ is biotinyl;
(b) compounds of the formula: 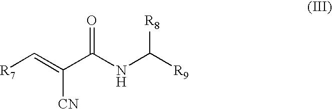
wherein:
R₇ is imidazolyl or: 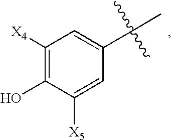
wherein:
X₄ and X₅ are each independently H, alkyl, alkenyl, alkynyl, aryl, aralkyl, acyl, alkoxy, alkenoxy, alkynyloxy, aryloxy, aralkyloxy, acyloxy, alkylamino, alkenylamino, alkynylamino, arylamino, aralkylamino, amido, alkylthio, alkenylthio, alkynylthio, arylthio, aralkylthio, acylthio, halo, hydroxy, amino, azido, mercapto, nitro, or cyano;
R₈ is H, alkyl, or phenyl; and
R₉ is phenyl or furanyl;
with the provisos that when R₇ is imidazolyl and R₈ is —CH₃, then R₉ is not —C₆H₅; and when X₄ is hydroxy, X₅ is H and R₈ is 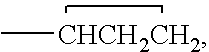
then R₉ is not —C₆H₅;
(c) compounds of the formula: 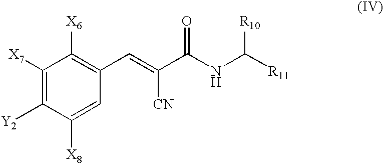
wherein:
R₁₀ is H, alkyl, or phenyl;
R₁₁ is phenyl or furanyl;
X₆ and X₇ are each independently H or nitro;
X₈ is H, halogen, or nitro; and
Y₂ is halogen or nitro;
with the provisos that when Y₂ is nitro, X₆, X₇ and X₈ are each H and R₁₀ is either H or —CH₃, then R₁₁ is not —C₆H₅;
when Y₂ is nitro, X₆ and X₇ are each H, X₈ is hydroxy and R₁₀ is —CH₃, then R₁₁ is not —C₆H₅; and
when Y₂ is chloro, X₆ and X₇ are each H, X₈ is nitro and R₁₀ is H, then R₁₁ is not —C₆H₅;
(d) compounds of the formula: 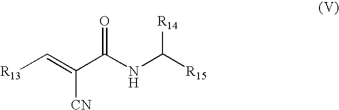
wherein:
R₁₃ is quinolinyl or ortho-bromopyridyl;
R₁₄ is selected from the group consisting of R_{A} and R_{B}-R_{C}, wherein:
R_{A} is selected from the group consisting of H, alkyl and phenyl;
R_{B} is alkanediyl; and
R_{C} is selected from the group consisting of phenyl, furanyl, acyl, acyloxy, hydroxy and biotinyl; or
R₁₄ taken together with R₁₅ forms 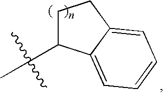
wherein n is 1-3; and
R₁₅ is furanyl or phenyl;
with the provisos that when R₁₃ is ortho-bromopyridyl and R₁₅ is —C₆H₅, then R₁₄ is not H, —CH₃, —CH₂CH₃, —CH₂CH₂CH₃, —C₆H₅, —CH₂C₆H₅, —CH₂OH, —CH₂OAc, —CH₂OC(O)CH(CH₃)₃, or 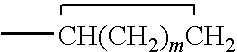
wherein m=1-3; and
(e) compounds of the formula: 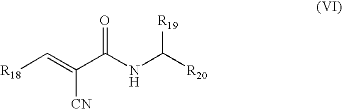
wherein:
R₁₈ is ortho-bromopyridyl or 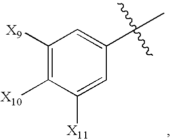
wherein:
X₉ and X₁₁ are each independently H or nitro; and
X₁₀ is H or chloro;
R₁₉ is —CH₃, —CH₂CH₃, —CH₂CH₂CH₃, —CH₂OH, —CH₂C₆H₅, —CH₂CO₂CH₃, —CH₂OC(O)CH₃, or 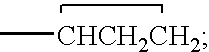
or
R₁₉ taken together with R₂₀ forms 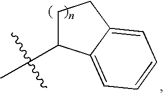
wherein n is 1-3; and
R₂₀ is 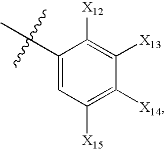
wherein:
X₁₂ is H or fluoro;
X₁₃ is H, —OCH₃, or fluoro;
X₁₄ is H, —CH₃, bromo, chloro, fluoro, or —OCH₃; and
X₁₅ is H or —CF₃;
with the provisos that when R₁₈ is ortho-bromopyridyl and R₂₀ is —C₆H₅, then R₁₉ is not —CH₃, —CH₂CH₃, —CH₂OH, or 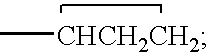
and
when R₁₈ is ortho-bromopyridyl, R₁₉ is H, then R₂₀ is not —C₆H₅.

2. The compound of claim 1, further defined as the compound of formula (II).

3. The compound of claim 2, wherein R₁ is selected from the group consisting of H, alkyl, alkoxy, acyl, N-piperidinyl,

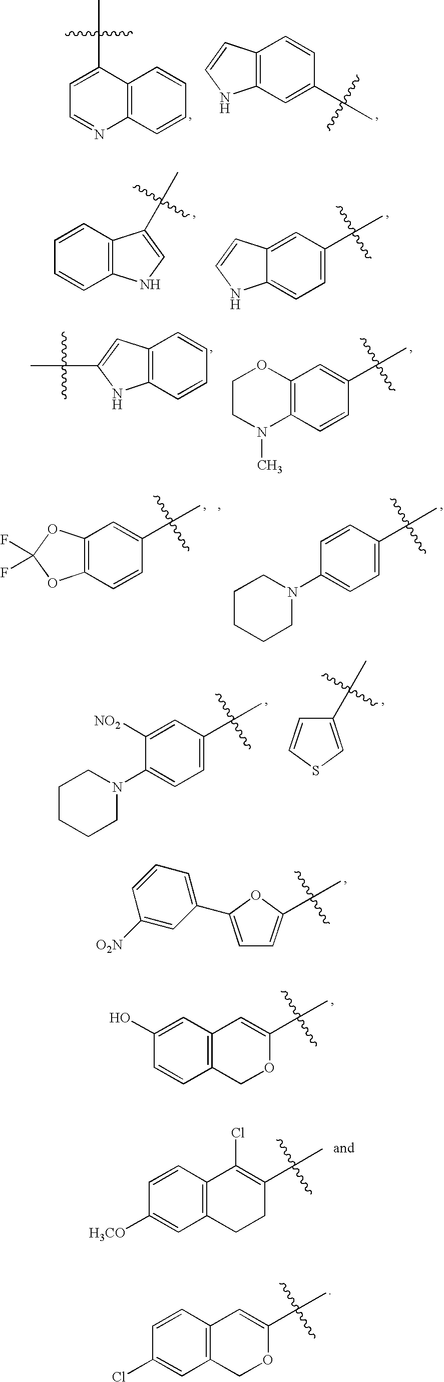

4. The compound of claim 2, wherein Y₁ is selected from the group consisting of H, n-hexyl, —OC₆H₁₃, —OCO₂CH₃, —OCH₃ and —OAc.

5. The compound of claim 2, wherein Y₁ is H and X₁, X₂, X₃ and X₄ are each independently selected from the group consisting of H, halo, hydroxy, nitro, —OCH₃, —OAc and —OC(O)OCH₃.

6. The compound of claim 5, wherein R₆ is mono-, di-, or tri-substituted phenyl.

7. The compound of claim 2, wherein R₅ is selected from the group consisting of H, —CH₃, —CH₂CH₃, —CH₂—CH₂CH₃, 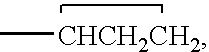
substituted or unsubstituted phenyl and methylfuranyl.

8. The compound of claim 2, wherein R₅ is biotinyl.

9-77. (canceled)

78. The compound of claim 1, further defined as the compound of formula (III).

79. The compound of claim 78, wherein R₇ is mono- or di-substituted phenyl.

80. The compound of claim 78, wherein X₄ and X₅ are independently selected from the group consisting of H, halo and nitro.

81. The compound of claim 78, wherein R₈ is selected from the group consisting of H, lower alkyl and —C₆H₅.

82. The compound of claim 78, wherein R₉ is selected from the group consisting of —C₆H₅, —C₆H₄Cl, —C₆H₄OCH₃ and methylfuranyl.

83-94. (canceled)

95. The compound of claim 1, further defined as the compound of formula (IV).

96. The compound of claim 95, wherein X₆, X₇ and X₈ are each independently selected from the group consisting of H, halogen and nitro.

97. The compound of claim 96, wherein X₆, X₇ and X₈ are each independently H or Cl.

98. The compound of claim 96, wherein X₆, X₇ and X₈ are each independently H or NO₂.

99. The compound of claim 95, wherein R₁₀ is selected from the group consisting of H, —CH₃, —CH₂CH₂CH₃, —CH₂OH and —C₆H₅.

100. The compound of claim 95, wherein R₁₁ is mono-substituted phenyl or furanyl.

101. The compound of claim 100, wherein R₁₁ is selected from the group consisting of —C₆H₄OCH₃—C₆H₄Cl, or methylfuranyl.

102-117. (canceled)

118. The compound of claim 1, further defined as the compound of formula (V).

119. The compound of claim 118, wherein R₁₃ is

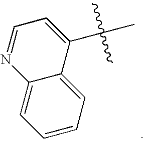

120. The compound of claim 118, wherein R₁₄ is selected from the group consisting of H, lower alkyl and phenyl.

121. The compound of claim 118, wherein R_{C} is selected from the group consisting of —CO₂CH₃, —OC(O)CH₃, —OC(O)benzophenone and —C₆H₅.

122. The compound of claim 119, wherein R₁₅ is selected from the group consisting of mono- or di-substituted phenyl and methylfuranyl.

123. The compound of claim 122, wherein R₁₅ is mono- or di-substituted with a substituent selected from the group consisting of H, —CH₃, —CF₃, halo, —OCH₃, azido and amino.

124-151. (canceled)

152. The compound of claim 1, further defined as the compound of formula (VI).

153. The compound of claim 152, wherein R₁₉ is selected from the group consisting of —CH₃, —CH₂CH₃ and —CH₂CH₂CH₃.

154. The compound of claim 152, wherein R₁₈ is ortho-bromopyridyl.

155. The compound of claim 152, wherein X₁₀ is H.

156. The compound of claim 152, wherein X₉, X₁₀ and X₁₁ are each H.

157. The compound of claim 152, wherein R₂₀ is selected from the group consisting of —C₆H₅ and mono- and di-substituted phenyl.

158. The compound of claim 1, wherein the compound is comprised in a pharmaceutically acceptable excipient, diluent, or vehicle.

159. A method of treating a cell proliferative disease comprising administering to a subject an amount of a first compound effective to treat the cell proliferative disease, wherein the first compound is a compound of claim 1.

160. The method of claim 159, wherein the subject is a mammal.

161. The method of claim 160, wherein the mammal is a human.

162. The method of claim 159, wherein the first compound is comprised in a pharmaceutically acceptable excipient, diluent, or vehicle.

163. The method of claim 159, wherein the cell proliferative disease is cancer.

164. The method of claim 163, wherein the cancer is melanoma, non-small cell lung, small cell lung, lung, hepatocarcinoma, retinoblastoma, astrocytoma, glioblastoma, leukemia, blood, brain, skin, eye, tongue, gum, neuroblastoma, head, neck, breast, pancreatic, renal, bone, testicular, ovarian, mesothelioma, cervical, gastrointestinal, lymphoma, colon, or bladder cancer.

165. The method of claim 159, wherein the cell proliferative disease is rheumatoid arthritis, inflammatory bowel disease, osteoarthritis, leiomyomas, adenomas, lipomas, hemangiomas, fibromas, vascular occlusion, restenosis, atherosclerosis, a pre-neoplastic lesion, carcinoma in situ, oral hairy leukoplakia, or psoriasis.

166. The method of claim 159, wherein c-myc expression is reduced in a cell of the subject.

167. The method of claim 159, wherein Jak2 expression is reduced in a cell of the subject.

168. The method of claim 159, wherein Stat3 expression is reduced in a cell of the subject.

169. The method of claim 159, wherein BCL-ABL expression is reduced in a cell of the subject.

170. The method of claim 159, wherein the first compound is administered in combination with a therapeutically relevant amount of a second compound.

171. The method of claim 170, wherein the second compound is an anti-cancer compound.

172. The method of claim 159, wherein the first compound is administered in combination with a surgery, a radiation therapy, or a gene therapy.
